(19) United States
(12) Patent Application Publication
(10) Pub. No.: US 2025/0250251 A1
(43) Pub. Date: Aug. 7, 2025

(54) PIPERIDINYLPYRIDINYLCARBONITRILE DERIVATIVES AS INHIBITORS OF GLUTAMINYL-PEPTIDE CYCLOTRANSFERASE AND GLUTAMINYL-PEPTIDE CYCLOTRANSFERASE LIKE PROTEIN

(71) Applicant: Boehringer Ingelheim International GmbH, Ingelheim am Rhein (DE)

(72) Inventors: Jens WILLWACHER, Biberach an der Riss (DE); Georg DAHMANN, Biberach (DE); Sandra Ruth HANDSCHUH, Biberach an der Riss (DE); Christian Andreas KUTTRUFF, Donaueschingen (DE); Sophia Astrid REINDL, Mittelbiberach (DE)

(21) Appl. No.: 19/041,039

(22) Filed: Jan. 30, 2025

(30) Foreign Application Priority Data
Feb. 6, 2024 (EP) 24156123.2

(51) Int. Cl. C07D 401/14; A61K 31/4545; A61K 31/501; A61K 31/506; C07D 471/04

(52) U.S. Cl. CPC C07D 401/14; A61K 31/4545; A61K 31/501; A61K 31/506; C07D 471/04

(57) ABSTRACT

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### TECHNICAL FIELD

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### BACKGROUND INFORMATION

Glutaminyl-peptide cyclotransferase (QPCT) and glutaminyl-peptide cyclotransferase-like protein (QPCTL) catalyze the intramolecular cyclization of N-terminal glutamine (Q) residues into pyroglutamic acid (pE) liberating ammonia [Stephan Schilling et al., “Identification of Human Glutaminyl Cyclase as a Metalloenzyme POTENT INHIBITION BY IMIDAZOLE DERIVATIVES AND HETEROCYCLIC CHELATORS,” Journal of Biological Chemistry 278, no. 50 (2003): 49773-79, https://doi.org/10.1074/jbc.m309077200; Holger Cynis et al., “Isolation of an Isoenzyme of Human Glutaminyl Cyclase: Retention in the Golgi Complex Suggests Involvement in the Protein Maturation Machinery,” Journal of Molecular Biology 379, no. 5 (2008): 966-80, https://doi.org/10.1016/j.jmb.2008.03.078; Anett Stephan et al., “Mammalian Glutaminyl Cyclases and Their Isoenzymes Have Identical Enzymatic Characteristics,” FEBS Journal 276, no. 22 (2009): 6522-36, https://doi.org/10.1111/j.1742-4658.2009.07337.x.]. While QPCT is a secreted protein, QPCTL is retained within the Golgi complex. Both enzymes share a high homology in the active site and similar catalytic specificity. Because of the high homology in the active site, inhibition of the active site blocks the enzymatic activity of both enzymes: QPCT and QPCTL. Hence the term “QPCT/L” describes both enzymes at once. Due to their different cellular localisation, differences in their relevance for modification of biological substrates have been reported. Known substrates of the intracellular QPCTL and/or extracellular QPCT are CD47 [Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.], different chemokines (like for example CCL2 and 7 or CX3CL1) [Rosa Barreira da Silva et al., “Loss of the Intracellular Enzyme QPCTL Limits Chemokine Function and Reshapes Myeloid Infiltration to Augment Tumor Immunity,” Nature Immunology 23, no. 4 (2022): 568-80, https://doi.org/10.1038/s41590-022-01153-x; Astrid Kehlen et al., “N-Terminal Pyroglutamate Formation in CX3CL1 Is Essential for Its Full Biologic Activity,” Bioscience Reports 37, no. 4 (2017): BSR20170712, https://doi.org/10.1042/bsr20170712.], Amyloid-b peptides [Cynis et al., “Isolation of an Isoenzyme of Human Glutaminyl Cyclase: Retention in the Golgi Complex Suggests Involvement in the Protein Maturation Machinery.”] or hormones like TRH [Andreas Becker et al., “IsoQC (QPCTL) Knock-out Mice Suggest Differential Substrate Conversion by Glutaminyl Cyclase Isoenzymes,” Biological Chemistry 397, no. 1 (2016): 45-55, https://doi.org/10.1515/hsz-2015-0192.]. The modification of N-terminal glutamine to pyroglutamate on the substrates has functional consequences for the proteins and could impact different pathomechanisms in several diseases. CD47 is expressed on the cell surface of virtually all cells of the body, including apoptotic cells, senescent cells or cancer cells. [Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011]. The main ligand for CD47 is signal-regulatory protein alpha (SIRPα), an inhibitory transmembrane receptor present on myeloid cells, such as macrophages, monocytes, neutrophils, dendritic cells and others. QPCTL mediated N-terminal pyroglutamate modification on CD47 is required for SIRPα binding [Deborah Hatherley et al., “Paired Receptor Specificity Explained by Structures of Signal Regulatory Proteins Alone and Complexed with CD47,” Molecular Cell 31, no. 2 (2008): 266-77, https://doi.org/10.1016/j.molcel.2008.05.026; Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.] This signaling axis induces a “Don't Eat Me Signal”, preventing engulfment of CD47 expressing cells by macrophages. Thus, high expression of CD47 is connected to the pathogenesis of cancer [Logtenberg et al., “Glutaminyl Cyclase Is an Enzymatic Modifier of the CD47-SIRPα Axis and a Target for Cancer Immunotherapy,” 2019; Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011.], COVID-19 [Katie-May Mclaughlin et al., “A Potential Role of the CD47/SIRPalpha Axis in COVID-19 Pathogenesis,” Current Issues in Molecular Biology 43, no. 3 (2021): 1212-25, https://doi.org/10.3390/cimb43030086.], lung fibrosis [Gerlinde Wernig et al., “Unifying Mechanism for Different Fibrotic Diseases,” Proceedings of the National Academy of Sciences 114, no. 18 (2017): 4757-62, https://doi.org/10.1073/pnas. 1621375114; Lu Cui et al., “Activation of JUN in Fibroblasts Promotes Pro-Fibrotic Programme and Modulates Protective Immunity,” Nature Communications 11, no. 1 (2020): 2795, https://doi.org/10.1038/s41467-020-16466-4.], systemic sclerosis [Wernig et al., “Unifying Mechanism for Different Fibrotic Diseases”; Tristan Lerbs et al., “CD47 Prevents the Elimination of Diseased Fibroblasts in Scleroderma,” JCI Insight 5, no. 16 (2020): 140458, https://doi.org/10.1172/jci.insight.140458.] and liver fibrosis [Taesik Gwag et al., “Anti-CD47 Antibody Treatment Attenuates Liver Inflammation and Fibrosis in Experimental Non-alcoholic Steatohepatitis Models,” Liver International 42, no. 4 (2022): 829-41, https://doi.org/10.1111/liv.15182.]. Since enhanced CD47 expression blocks the clearance of apoptotic cells, there is an accrual of apoptotic lung epithelial cells, leading to a pro-fibrotic stimulus and accelerating lung inflammation and -scaring [Alexandra L. McCubbrey and Jeffrey L. Curtis, “Efferocytosis and Lung Disease,” Chest 143, no. 6 (2013): 1750-57, https://doi.org/10.1378/chest.12-2413; Brennan D. Gerlach et al., “Efferocytosis Induces Macrophage Proliferation to Help Resolve Tissue Injury,” Cell Metabolism, 2021, https://doi.org/10.1016/j.cmet.2021.10.015.]. Since CD47 half-life and function is majorly dependent on QPCTL enzyme activity, QPCT and QPCTL inhibition could be a suitable mechanism as a treatment in lung fibrosis such as IPF or SSC-ILD [Lerbs et al., “CD47 Prevents the Elimination of Diseased Fibroblasts in Scleroderma.”], alone or together with current standard of care in pulmonary fibrosis like Nintedanib [Luca Richeldi et al., “Efficacy and Safety of Nintedanib in Idiopathic Pulmonary Fibrosis,” The New England Journal of Medicine 370, no. 22 (2014): 2071-82, https://doi.org/10.1056/nejmoa1402584; Kevin R Flaherty et al., “Nintedanib in Progressive Fibrosing Interstitial Lung Diseases,” New England Journal Medicine (2019): 1718-27, of 381, no. 18 https://doi.org/10.1056/nejmoa1908681.] or future treatments like a PDE4 inhibitor [Luca Richeldi et al., “Trial of a Preferential Phosphodiesterase 4B Inhibitor for Idiopathic Pulmonary Fibrosis,” New England Journal of Medicine 386, no. 23 (2022): 2178-87, https://doi.org/10.1056/nejmoa2201737].

By expression of CD47, cancer cells can evade destruction by the immune system or evade immune surveillance, e.g. by evading phagocytosis by immune cells [Stephen B. Willingham et al., “The CD47-Signal Regulatory Protein Alpha (SIRPα) Interaction Is a Therapeutic Target for Human Solid Tumors,” Proceedings of the National Academy of Sciences 109, no. 17 (2012): 6662-67, https://doi.org/10.1073/pnas.1121623109].

In addition to CD47, chemokines, such as CCL2 and CX3CL1, have been identified as QPCTL and/or QPCT substrates [Holger Cynis et al., “The Isoenzyme of Glutaminyl Cyclase Is an Important Regulator of Monocyte Infiltration under Inflammatory Conditions,” EMBO Molecular Medicine 3, no. 9 (2011): 545-58, https://doi.org/10.1002/emmm.201100158]. The formation of the N-terminal pGlu was shown to increase in vivo activity, both by conferring resistance to aminopeptidases and by increasing its capacity to induce chemokine receptor signaling. Two main monocyte chemoattractants CCL2 and CCL7 are insensitive to DPP4-inactivation in vivo because of an intracellular mechanism of N-terminal cyclization mediated by the Golgi-associated enzyme QPCTL. It has been shown that QPCTL is a critical regulator of monocyte migration into solid tumors [Kaspar Bresser et al., “QPCTL Regulates Macrophage and Monocyte Abundance and Inflammatory Signatures in the Tumor Microenvironment,” Oncoimmunology 11, no. 1 (2022): 2049486, https://doi.org/10.1080/2162402x.2022.2049486; Rosa Barreira da Silva et al., “Loss of the Intracellular Enzyme QPCTL Limits Chemokine Function and Reshapes Myeloid Infiltration to Augment Tumor Immunity,” Nature Immunology, 2022, 1-13, https://doi.org/10.1038/s41590-022-01153-x]. Targeting of chemokines has long been pursued as a potential strategy for modulating cellular trafficking in different disease settings.

It is therefore desirable to provide potent QPCT/L inhibitors.

WO 2023/205173 discloses QPCTL modulators of the general formula:

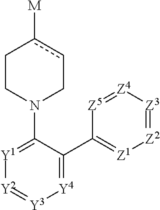

which includes compound 14:

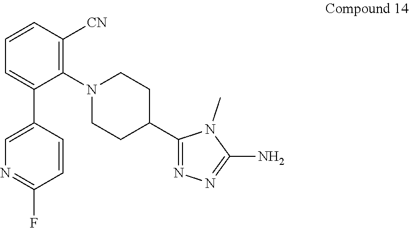 [SMILES: Cn1c(N)nnc1C1CCN(c2c(C#N)cccc2-c2ccc(F)nc2)CC1]

Compound 14 in WO 2023/205173 is disclosed therein as having inhibitory activity on isolated QPCTL of IC₅₀<1 μM and cellular (A549) activity of EC₅₀<1 μM.

Yu, L., Zhao, P., Sun, Y. et al. Sig Transduct Target Ther 8, 454 (2023) (herein “STTT 2023”) disclose compounds QP5020 and QP5038 as potent benzonitrile-based inhibitors of glutaminyl-peptide cyclotransferase-like protein (QPCTL) with antitumor efficacy:

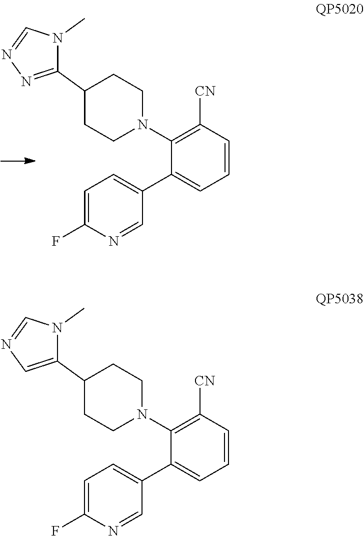 [SMILES: Cn1cnnc1C1CCN(c2c(C#N)cccc2-c2ccc(F)nc2)CC1 ; Cn1cncc1C1CCN(c2c(C#N)cccc2-c2ccc(F)nc2)CC1]

Compound QP5020 is disclosed therein as having QPCTL inhibition activity of IC₅₀ 15.0+/−5.5 nM and QP5038 as having QPCTL inhibition activity of IC₅₀ 3.8+/−0.7 nM.

WO 2024/020517 discloses inhibitors of general formula:

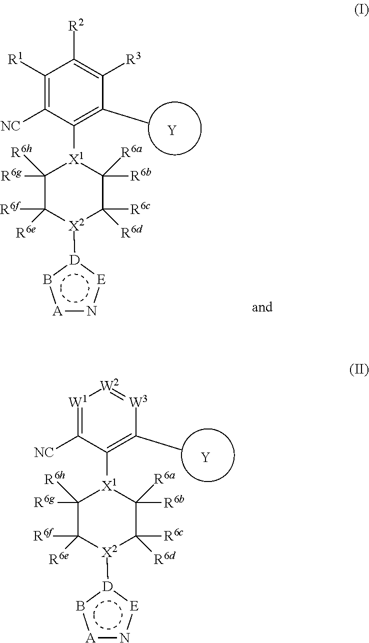

which includes compound (1):

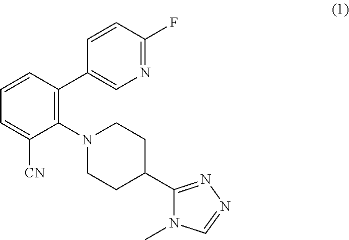 [SMILES: Cn1cnnc1C1CCN(c2c(C#N)cccc2-c2ccc(F)nc2)CC1]

Compound (1) in WO 2024/020517 is disclosed therein as having inhibitory activity on isolated QPCTL of IC₅₀<0.1 μM and cellular (Ramos) activity of IC₅₀<0.1 μM. Compound (1) in WO 2024/020517 and QP5020 are identical.

Further selected examples in WO 2024/020517 are:

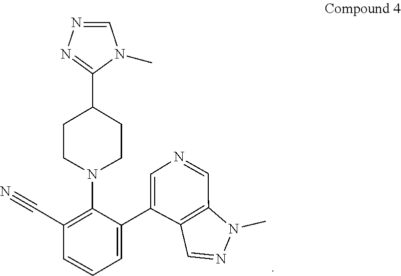 [SMILES: Cn1cnnc1C1CCN(c2c(C#N)cccc2-c2cncc3c2cnn3C)CC1]

Inhibitory activity on isolated QPCTL of IC₅₀>0.1 μM to ≤1.0 μM;

Inhibitory activity of cellular (Ramos) QPCTL activity in a FACS assay of >1 μM to ≤10 μM.

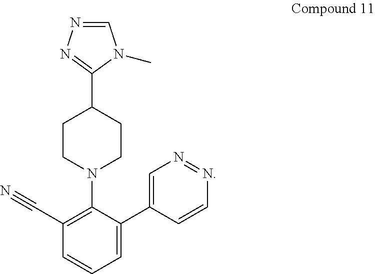 [SMILES: Cn1cnnc1C1CCN(c2c(C#N)cccc2-c2ccnnc2)CC1]

Inhibitory activity on isolated QPCTL of IC₅₀<0.1 μM;

Inhibitory activity of cellular (Ramos) QPCTL activity in a FACS assay of <0.1 μM.

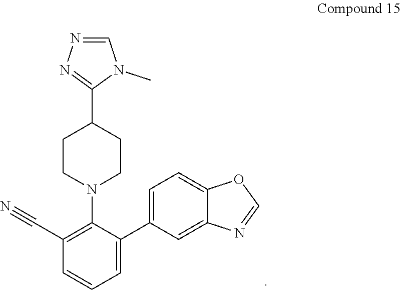 [SMILES: Cn1cnnc1C1CCN(c2c(C#N)cccc2-c2ccc3ocnc3c2)CC1]

Inhibitory activity on isolated QPCTL of IC₅₀>0.1 μM and <1.0 M;

Inhibitory activity of cellular (Ramos) QPCTL activity in a FACS assay of >1.0 μM to ≤10 μM.

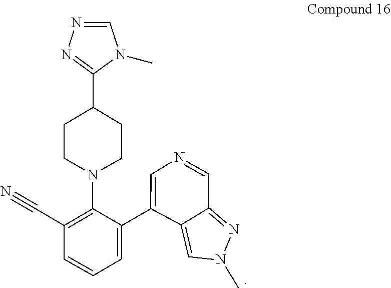 [SMILES: Cn1cc2c(-c3cccc(C#N)c3N3CCC(c4nncn4C)CC3)cncc2n1]

Inhibitory activity on isolated QPCTL of IC₅₀<0.1 μM;

Inhibitory activity of cellular (Ramos) QPCTL activity in a FACS assay of >0.1 μM to ≤1.0 μM.

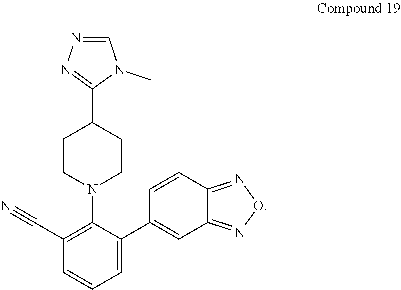 [SMILES: Cn1cnnc1C1CCN(c2c(C#N)cccc2-c2ccc3nonc3c2)CC1]

Inhibitory activity on isolated QPCTL of IC₅₀>0.1 μM and <1 μM;

Inhibitory activity of cellular (Ramos) QPCTL activity in a FACS assay of >1 μM to ≤10 HM.

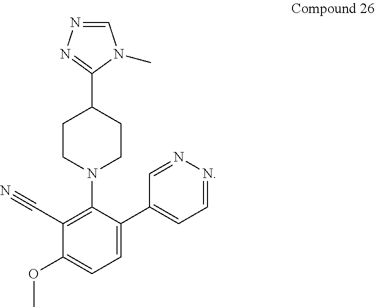 [SMILES: COc1ccc(-c2ccnnc2)c(N2CCC(c3nncn3C)CC2)c1C#N]

Inhibitory activity on isolated QPCTL of IC₅₀<0.1 μM;

Inhibitory activity of cellular (Ramos) QPCTL activity in a FACS assay of <0.1 μM.

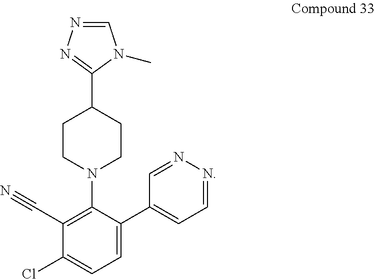 [SMILES: Cn1cnnc1C1CCN(c2c(-c3ccnnc3)ccc(Cl)c2C#N)CC1]

Inhibitory activity on isolated QPCTL of IC₅₀<0.1 μM;

Inhibitory activity of cellular (Ramos) QPCTL activity in a FACS assay of <0.1 μM.

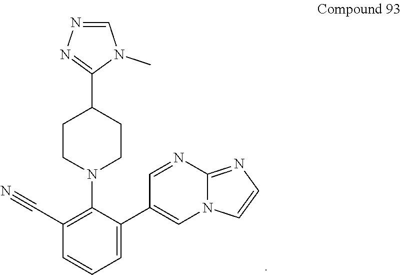 [SMILES: Cn1cnnc1C1CCN(c2c(C#N)cccc2-c2cnc3nccn3c2)CC1]

Inhibitory activity on isolated QPCTL of IC₅₀<0.1 μM;

Inhibitory activity of DLD-1 cellular QPCTL activity in an imaging assay of >0.1 μM to ≤1.0 μM.

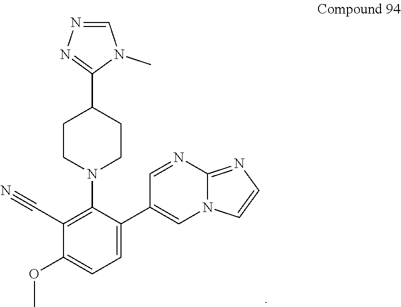 [SMILES: COc1ccc(-c2cnc3nccn3c2)c(N2CCC(c3nncn3C)CC2)c1C#N]

Inhibitory activity on isolated QPCTL of IC₅₀<0.1 μM;

Inhibitory activity of DLD-1 cellular QPCTL activity in an imaging assay of >1 μM to ≤10 μM.

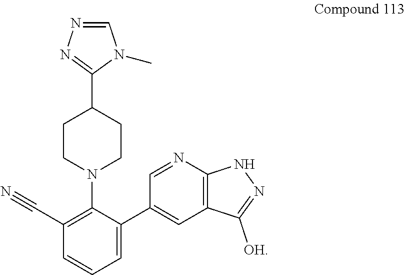 [SMILES: Cn1cnnc1C1CCN(c2c(C#N)cccc2-c2cnc3[nH]nc(O)c3c2)CC1]

Inhibitory activity on isolated QPCTL of IC₅₀<0.1 μM.

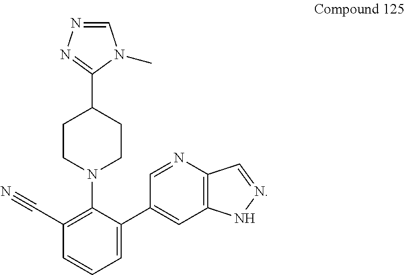 [SMILES: Cn1cnnc1C1CCN(c2c(C#N)cccc2-c2cnc3cn[nH]c3c2)CC1]

Inhibitory activity on isolated QPCTL of IC₅₀>0.1 μM to ≤1.0 μM.

### LEGEND TO THE FIGURES

FIG. 1—Introduction of the fluoro substituent on the piperidyl ring at the 4-position leads to increased permeability for QPCT/L inhibitors bearing a pyrido- or benzonitrile core.

FIG. 2—Introduction of the fluoro substituent on the piperidyl ring at the 4-position leads to decreased efflux ratio for QPCT/L inhibitors bearing a pyrido- or benzonitrile core.

### DETAILED DESCRIPTION OF THE INVENTION

The present invention discloses novel piperidinylpyridinylcarbonitrile derivatives of formula (I)

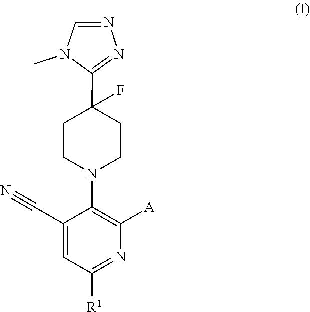

that are inhibitors of Glutaminyl-peptide cyclotransferase (QPCT) and glutaminyl-peptide cyclotransferase-like protein (QPCTL), possessing appropriate pharmacological and pharmacokinetic properties enabling their use as medicaments for the treatment of conditions and/or diseases treatable by inhibition of QPCT/L.

The compounds of the present invention may provide several advantages, such as enhanced potency, cellular potency, high metabolic and/or chemical stability, high selectivity, safety and tolerability, enhanced solubility, enhanced permeability, desirable plasma protein binding, enhanced bioavailability, suitable pharmacokinetic profiles, and the possibility to form stable salts.

### Compounds of the Invention

The present invention provides novel piperidinylpyridinylcarbonitrile derivatives that surprisingly, are potent inhibitors of QPCT and QPCTL (Assay A), as well as potent inhibitors of QPCT/L in cells relevant for, but not limited to, lung diseases or cancer, (Assay B).

Furthermore, the present novel piperidinylpyridinylcarbonitrile derivatives have appropriate membrane permeability and a low in vitro efflux (Assay C).

Furthermore, the compounds of the present invention have a favorable CYP induction profile as indicated by a low n-fold induction of CYP3A4 mRNA after incubation with the compound at 10 UM concentration (Assay D).

Furthermore, the compounds of the present invention show improved stability in murine hepatocytes that facilitates preclinical compound evaluation (Assay E).

Compounds of the present invention bear a fluoro substituent attached to the 4-position of the piperidyl ring (noted herein below as “4-fluoropiperidyl” in the tables), which show surprisingly higher permeability in CACO2-Cells and reduced efflux, (Assay C). This effect is demonstrated with the following comparisons with the analogous non-fluoro compound:

| Compound without 4-fluoropiperidyl | Compound with 4-fluoropiperidyl |
|---|---|
| 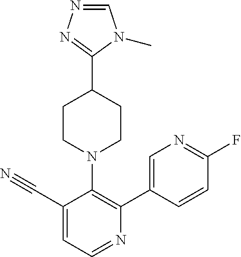 [SMILES: Cn1cnnc1C1CCN(c2c(C#N)ccnc2-c2ccc(F)nc2)CC1] Compound 133 in WO 2024/020517 CACO2 Perm. 4.8 x 10⁻⁶ cm/sec Efflux ratio (BA/AB) 3.5 | 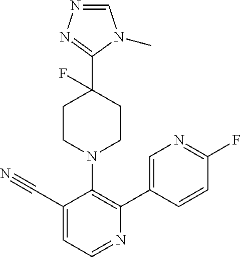 [SMILES: Cn1cnnc1C1(F)CCN(c2c(C#N)ccnc2-c2ccc(F)nc2)CC1] Example 1 in EP 22188580.9 CACO2 Perm. 28.0 x 10⁻⁶ cm/sec Efflux ratio (BA/AB) 2.3 |
| 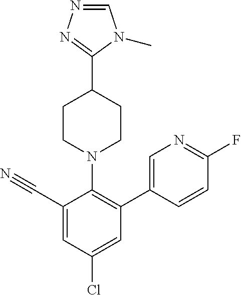 [SMILES: Cn1cnnc1C1CCN(c2c(C#N)cc(Cl)cc2-c2ccc(F)nc2)CC1] Compound 18 in WO 2024/020517 CACO2 Perm. 23.0 x 10⁻⁶ cm/sec Efflux ratio (BA/AB) 3.3 | 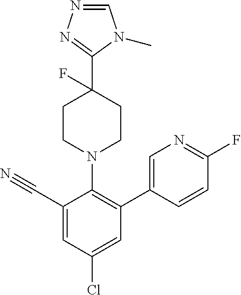 [SMILES: Cn1cnnc1C1(F)CCN(c2c(C#N)cc(Cl)cc2-c2ccc(F)nc2)CC1] Example 18 in EP 23161417.3 CACO2 Perm. 66.0 x 10⁻⁶ cm/sec Efflux ratio (BA/AB) 1.0 |
| 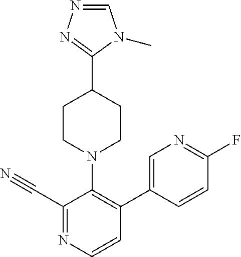 [SMILES: Cn1cnnc1C1CCN(c2c(-c3ccc(F)nc3)ccnc2C#N)CC1] Compound 132 in WO 2024/020517 CACO2 Perm. 1.8 x 10⁻⁶ cm/sec Efflux ratio (BA/AB) 7.8 | 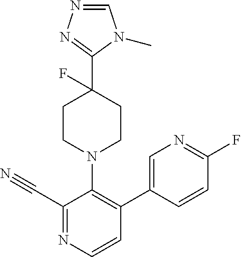 [SMILES: Cn1cnnc1C1(F)CCN(c2c(-c3ccc(F)nc3)ccnc2C#N)CC1] Example 10 in EP 22188580.9 CACO2 Perm. 11.0 x 10⁻⁶ cm/sec Efflux ratio (BA/AB) 5.6 |
| 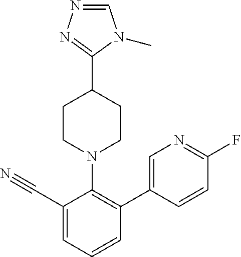 [SMILES: Cn1cnnc1C1CCN(c2c(C#N)cccc2-c2ccc(F)nc2)CC1] QP5020/Compound 1 in WO 2024/020517 CACO2 Perm. 28.0 x 10⁻⁶ cm/sec Efflux ratio (BA/AB) 2.4 | 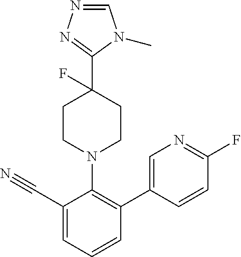 [SMILES: Cn1cnnc1C1(F)CCN(c2c(C#N)cccc2-c2ccc(F)nc2)CC1] Example 56 in EP 23161417.3 CACO2 Perm. 50.0 x 10⁻⁶ cm/sec Efflux ratio (BA/AB) 0.7 |
| 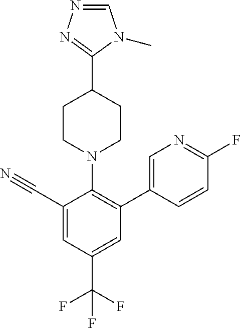 [SMILES: Cn1cnnc1C1CCN(c2c(C#N)cc(C(F)(F)F)cc2-c2ccc(F)nc2)CC1] Compound 70 in WO 2024/020517 CACO2 Perm. 9.0 x 10⁻⁶ cm/sec Efflux ratio (BA/AB) 10.4 | 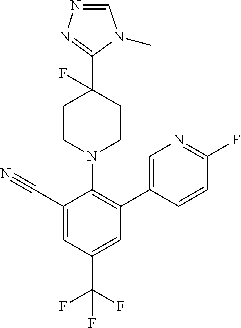 [SMILES: Cn1cnnc1C1(F)CCN(c2c(C#N)cc(C(F)(F)F)cc2-c2ccc(F)nc2)CC1] Example 4 in EP 23161417.3 CACO2 Perm. 40.5 x 10⁻⁶ cm/sec Efflux ratio (BA/AB) 1.3 |
| 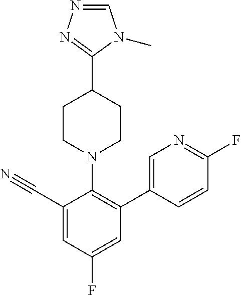 [SMILES: Cn1cnnc1C1CCN(c2c(C#N)cc(F)cc2-c2ccc(F)nc2)CC1] Compound 130 in WO 2024/020517 CACO2 Perm. 24.0 x 10⁻⁶ cm/sec Efflux ratio (BA/AB) 2.1 | 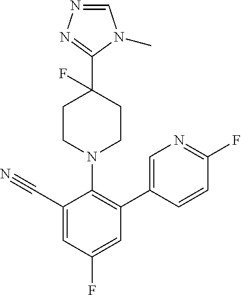 [SMILES: Cn1cnnc1C1(F)CCN(c2c(C#N)cc(F)cc2-c2ccc(F)nc2)CC1] Example 6 in EP 23161417.3 CACO2 Perm. 44.0 x 10⁻⁶ cm/sec Efflux ratio (BA/AB) 1.6 |
| 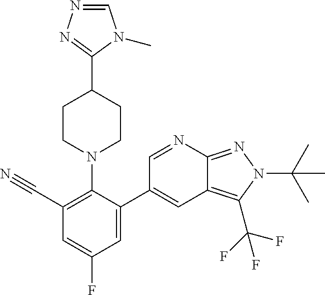 [SMILES: Cn1cnnc1C1CCN(c2c(C#N)cc(F)cc2-c2cnc3nn(C(C)(C)C)c(C(F)(F)F)c3c2)CC1] Hitherto unpublished reference compound CACO2 Perm. 17.0 x 10⁻⁶ cm/sec Efflux ratio (BA/AB) 5.7 | 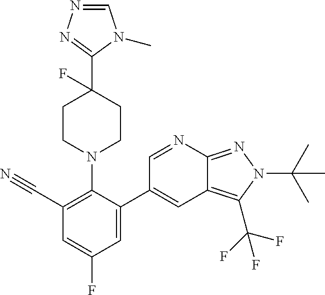 [SMILES: Cn1cnnc1C1(F)CCN(c2c(C#N)cc(F)cc2-c2cnc3nn(C(C)(C)C)c(C(F)(F)F)c3c2)CC1] Hitherto unpublished reference compound CACO2 Perm. 51.0 x 10⁻⁶ cm/sec Efflux ratio (BA/AB) 0.8 |
| 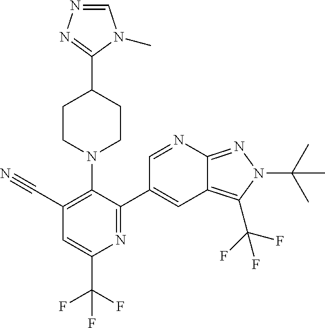 [SMILES: Cn1cnnc1C1CCN(c2c(C#N)cc(C(F)(F)F)nc2-c2cnc3nn(C(C)(C)C)c(C(F)(F)F)c3c2)CC1] Example 13 in EP 23189886.7 CACO2 Perm. 3.7 x 10⁻⁶ cm/sec Efflux ratio (BA/AB) 5.7 | 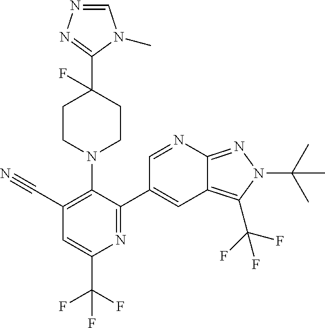 [SMILES: Cn1cnnc1C1(F)CCN(c2c(C#N)cc(C(F)(F)F)nc2-c2cnc3nn(C(C)(C)C)c(C(F)(F)F)c3c2)CC1] Example 4 in EP 23189886.7 CACO2 Perm. 99.5 x 10⁻⁶ cm/sec Efflux ratio (BA/AB) 0.6 |
| 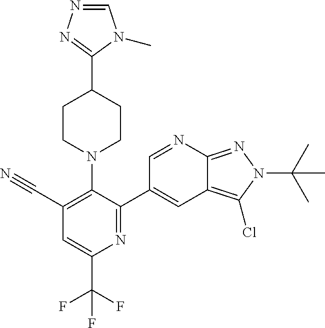 [SMILES: Cn1cnnc1C1CCN(c2c(C#N)cc(C(F)(F)F)nc2-c2cnc3nn(C(C)(C)C)c(Cl)c3c2)CC1] Hitherto unpublished reference compound CACO2 Perm. 1.0 x 10⁻⁶ cm/sec Efflux ratio (BA/AB) 36.4 | 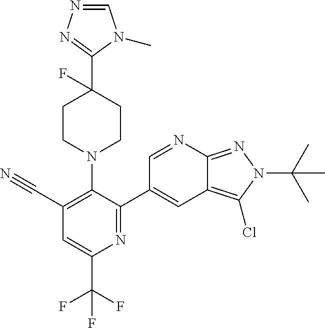 [SMILES: Cn1cnnc1C1(F)CCN(c2c(C#N)cc(C(F)(F)F)nc2-c2cnc3nn(C(C)(C)C)c(Cl)c3c2)CC1] Example 3 in EP 23189886.7 CACO2 Perm. 47.0 x 10⁻⁶ cm/sec Efflux ratio (BA/AB) 1.0 |
| 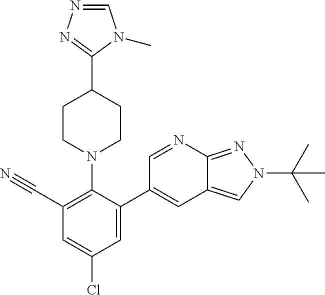 [SMILES: Cn1cnnc1C1CCN(c2c(C#N)cc(Cl)cc2-c2cnc3nn(C(C)(C)C)cc3c2)CC1] Example 17 in EP 23161417.3 CACO2 Perm. 2.5 x 10⁻⁶ cm/sec Efflux ratio (BA/AB) 21.2 | 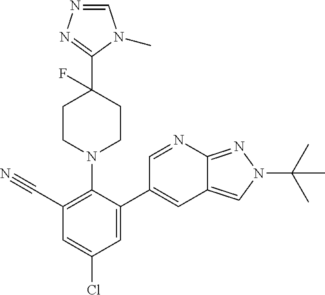 [SMILES: Cn1cnnc1C1(F)CCN(c2c(C#N)cc(Cl)cc2-c2cnc3nn(C(C)(C)C)cc3c2)CC1] Example 19 in EP 23161417.3 CACO2 Perm. 18.0 x 10⁻⁶ cm/sec Efflux ratio (BA/AB) 4.5 |
| 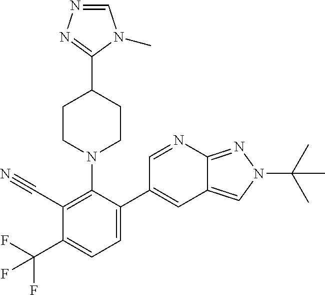 [SMILES: Cn1cnnc1C1CCN(c2c(-c3cnc4nn(C(C)(C)C)cc4c3)ccc(C(F)(F)F)c2C#N)CC1] Example 32 in EP 23161417.3 CACO2 Perm. 0.2 x 10⁻⁶ cm/sec Efflux ratio (BA/AB) 145.8 | 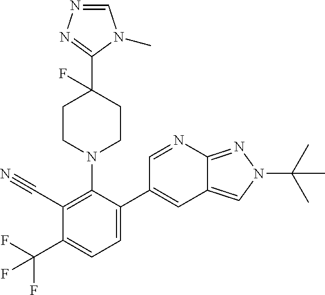 [SMILES: Cn1cnnc1C1(F)CCN(c2c(-c3cnc4nn(C(C)(C)C)cc4c3)ccc(C(F)(F)F)c2C#N)CC1] Example 30 in EP 23161417.3 CACO2 Perm. 1.7 x 10⁻⁶ cm/sec Efflux ratio (BA/AB) 22.9 |
| 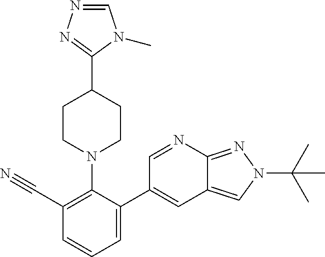 [SMILES: Cn1cnnc1C1CCN(c2c(C#N)cccc2-c2cnc3nn(C(C)(C)C)cc3c2)CC1] Example 50 in EP 22216126.7 CACO2 Perm. 0.5 x 10⁻⁶ cm/sec Efflux ratio (BA/AB) 75.5 | 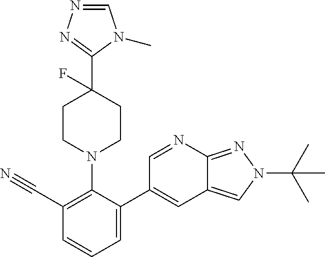 [SMILES: Cn1cnnc1C1(F)CCN(c2c(C#N)cccc2-c2cnc3nn(C(C)(C)C)cc3c2)CC1] Example 62 in EP 22216126.7 CACO2 Perm. 4.9 x 10⁻⁶ cm/sec Efflux ratio (BA/AB) 19.0 |
| 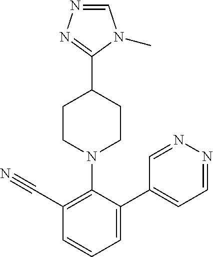 [SMILES: Cn1cnnc1C1CCN(c2c(C#N)cccc2-c2ccnnc2)CC1] Compound 11 in WO 2024/020517 CACO2 Perm. 0.3 x 10⁻⁶ cm/sec Efflux ratio (BA/AB) 28.1 | 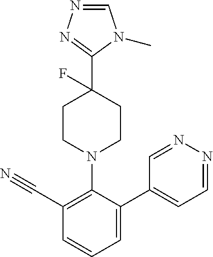 [SMILES: Cn1cnnc1C1(F)CCN(c2c(C#N)cccc2-c2ccnnc2)CC1] Example 52 in EP 22216126.7 CACO2 Perm. 15.0 x 10⁻⁶ cm/sec Efflux ratio (BA/AB) 1.1 |

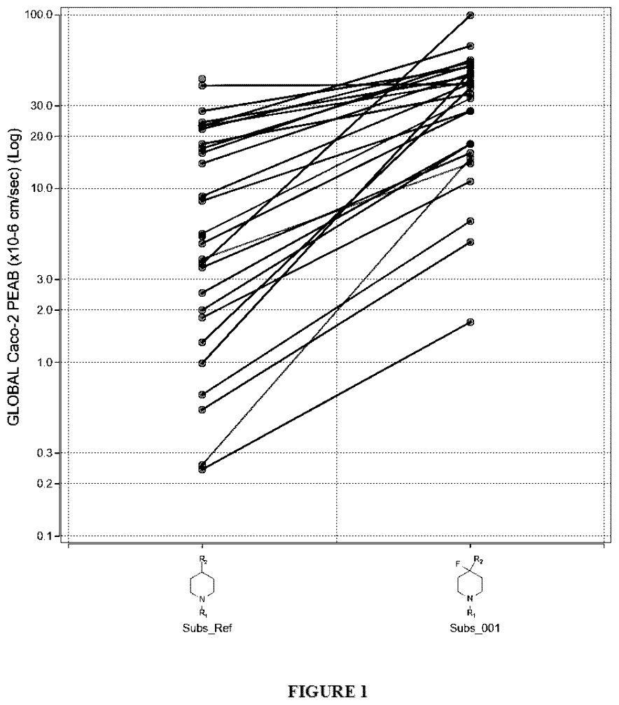

The increase in permeability in CACO2-Cells between the compared pairs of compounds is depicted in FIG. 1.

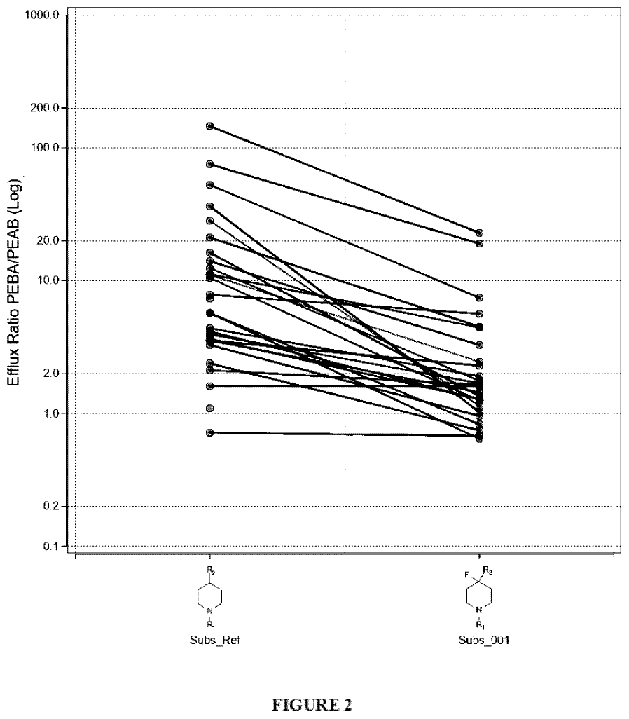

The decrease in efflux between the compared pairs of compounds is depicted in FIG. 2.

Consequently, compounds of the present invention are more viable for human use.

Compounds of the present invention differ structurally from Compound 14 in WO 2023/205173 in that the triazolyl ring in the 4-position of the piperidyl ring does not contain an amino substituent. Furthermore, the 4-position of the piperidyl ring is further substituted with fluorine. Still furthermore, the ring attached to the 1-position of the piperidyl ring is pyridyl, and said pyridyl ring has four substituents.

Compounds of the present invention differ structurally from the compounds in WO 2024/020517 including QP5020/Compound (1) in that the 4-position of the piperidyl ring is substituted with fluoro in addition to the triazolyl ring. Furthermore, the pyridyl ring attached to the 1-position of the piperidyl ring has four substituents, with one substituent at the para-position relative to the piperidyl ring. This differs to compounds disclosed in WO 2024/020517 which have three substituents or a fourth substituent at the meta-position relative to the piperidyl ring, such as example 94.

Furthermore, substituent A in the present invention is a pyrazolopyridinyl ring or a pyridazinyl ring attached to the pyridyl ring, whereas in WO 2024/020517 in the case of the pyrazolopyridinyl ring, regioisomers thereof are disclosed, such as Compound 16, Compound 93, Compound 94, Compound 113 and Compound 125, and said regioisomers or pyridazinyl ring are attached to a phenyl ring instead of a pyridyl ring as in Compound 11.

These structural differences between compounds of the present invention and the prior art unexpectedly lead to a favourable combination of (i) potent inhibition of QPCT and QPCTL, (ii) potent inhibition of QPCT/L in cells relevant for, but not limited to, lung diseases or cancer, (iii) appropriate membrane permeability and a low in vitro efflux, (iv) no or still acceptable induction of CYP3A4 mRNA levels and (v) improved stability in murine hepatocytes which facilitates preclinical compound evaluation and selection.

Compounds of the invention are thus superior to those disclosed in the prior art in terms of the combination of the following parameters:
potent inhibition of QPCT and QPCTL (Assay A)
potent inhibition of QPCT/L in cells relevant for, but not limited to, lung diseases or cancer (Assay B)
appropriate membrane permeability and a low in vitro efflux (Assay C)
no or still acceptable induction of CYP3A4 mRNA levels (Assay D)
improved stability in murine hepatocytes which facilitates preclinical compound evaluation and selection (Assay E)

The present invention provides novel compounds according to formula (I)

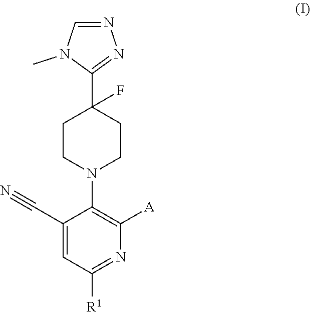

wherein
A is

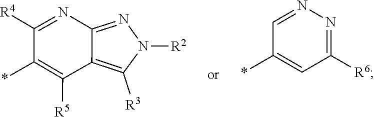

R¹ is selected from the group consisting of C₁₋₄-alkyl, F₁₋₉-fluoro-C₁₋₄-alkyl, C₃₋₆-cycloalkyl, F₁₋₈-fluoro-C₃₋₅-cycloalkyl,
or R¹ is selected from the group consisting of phenyl, pyridyl, pyrimidyl, pyrazolyl or oxazolyl; unsubstituted or substituted with one, two or three R⁷;
R² is selected from the group consisting of C₁₋₆-alkyl and F₁₋₉-fluoro-C₁₋₆-alkyl;
R³ is selected from the group consisting of H, C₁₋₆-alkyl, F₁₋₉-fluoro-C₁₋₆-alkyl and C₃₋₆-cycloalkyl;
R⁴ is selected from H or CHF₂;
R⁵ is selected from H or CHF₂;
R⁶ is selected from H or C₁₋₄-alkyl;
R⁷ is selected from C₁₋₄-alkyl, F₁₋₉-fluoro-C₁₋₆-alkyl or fluoro;
or a salt thereof, particularly a pharmaceutically acceptable salt thereof.

Another embodiment of the present invention relates to a compound of formula (I),
wherein A is

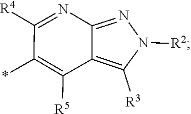

and substituents R¹, R², R³, R⁴, R⁵ and R⁷ are defined as in any of the preceding embodiments.

Another embodiment of the present invention relates to a compound of formula (I),
wherein A is

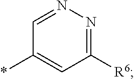

and substituents R¹, R⁶ and R⁷ are defined as in any of the preceding embodiments.

Another embodiment of the present invention relates to a compound of formula (I),
wherein R¹ is selected from the group R1b, consisting of C₁₋₄-alkyl, F₁₋₉-fluoro-C₁₋₄-alkyl, C₃₋₆-cycloalkyl, F₁₋₈-fluoro-C₃₋₅-cycloalkyl,
or R¹ is selected from the group consisting of

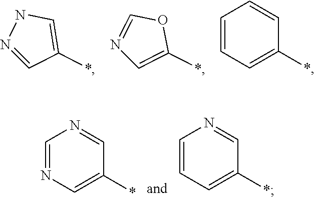

unsubstituted or substituted with one, two or three R⁷ and substituents R², R³, R⁴, R⁵, R⁶ and R⁷ are defined as in any of the preceding embodiments.

Another embodiment of the present invention relates to a compound of formula (I),
wherein R¹ is selected from the group R1c, consisting of methyl, —CF₂CH₃, CF₃, cyclopropyl,

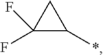

or R¹ is selected from the group consisting of

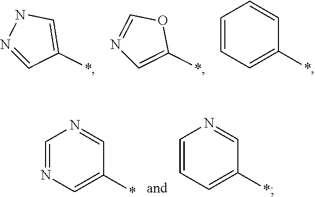

unsubstituted or substituted with one, two or three R⁷ and substituents R², R³, R⁴, R⁵, R⁶ and R⁷ are defined as in any of the preceding embodiments.

Another embodiment of the present invention relates to a compound of formula (I),
wherein R¹ is selected from the group R1d, consisting of methyl, —CF₂CH₃, CF₃, cyclopropyl,

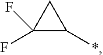

or R¹ is selected from the group consisting of

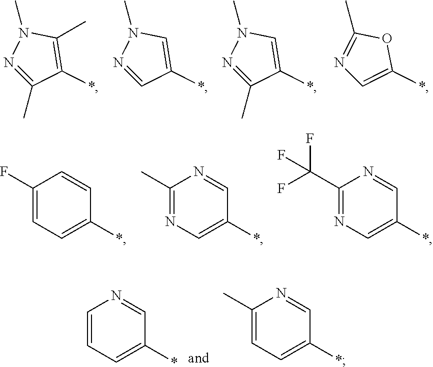

and substituents R², R³, R⁴, R⁵ and R⁶ are defined as in any of the preceding embodiments.

Another embodiment of the present invention relates to a compound of formula (I),
wherein R² is selected from the group R2b, consisting of methyl, CF₃ and t-butyl; and substituents R¹, R³, R⁴, R⁵, R⁶, and R⁷ are defined as in any of the preceding embodiments.

Another embodiment of the present invention relates to a compound of formula (I),
wherein R³ is selected from the group R3b, consisting of H, —CHF₂, CF₃ and cyclopropyl;
and substituents R¹, R², R⁴, R⁵, R⁶, and R⁷ are defined as in any of the preceding embodiments.

Another embodiment of the present invention relates to a compound of formula (I),
wherein R⁶ is selected from the group R6b, consisting of H and methyl;
and substituents R¹, R², R³, R⁴, R⁵, and R⁷ are defined as in any of the preceding embodiments.

Another embodiment of the present invention relates to a compound of formula (I),
wherein R⁷ is selected from the group R7b, consisting of methyl, fluoro and CF₃;
and substituents R¹, R², R³, R⁴, R⁵, and R⁶ are defined as in any of the preceding embodiments.

Another embodiment of the present invention relates to a compound of formula (I) above, having formula (I-a)

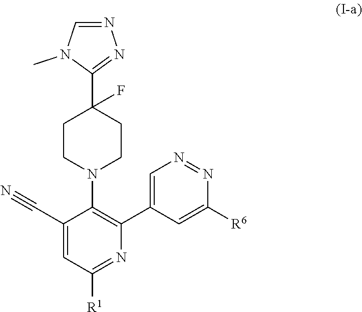

wherein substituents R¹, R⁵ and R⁶ are defined as in any of the preceding embodiments.

Another embodiment of the present invention relates to a compound of formula (I) above, having formula (I-b)

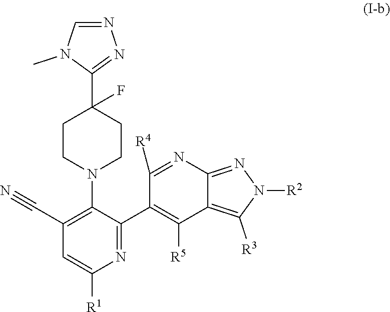

wherein substituents R¹, R², R³, R⁴ and R⁵ are defined as in any of the preceding embodiments.

Particularly preferred is the compound according to formula (I) selected from the group consisting of

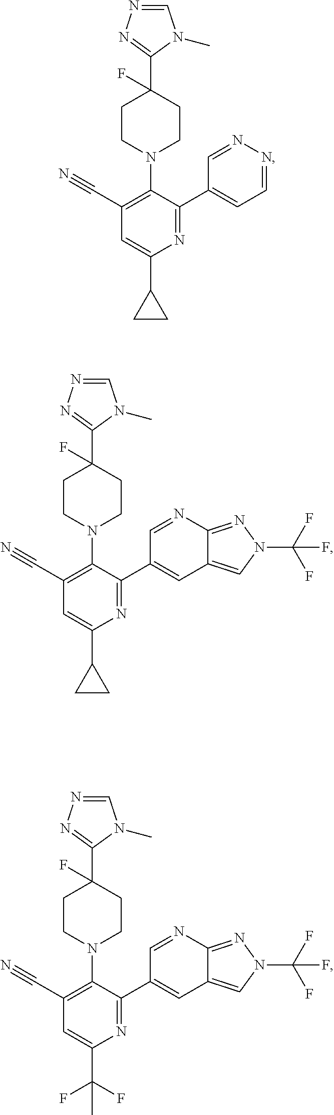 [SMILES: Cn1cnnc1C1(F)CCN(c2c(C#N)cc(C3CC3)nc2-c2ccnnc2)CC1 ; Cn1cnnc1C1(F)CCN(c2c(C#N)cc(C3CC3)nc2-c2cnc3nn(C(F)(F)F)cc3c2)CC1 ; Cn1cnnc1C1(F)CCN(c2c(C#N)cc(C(C)(F)F)nc2-c2cnc3nn(C(F)(F)F)cc3c2)CC1]

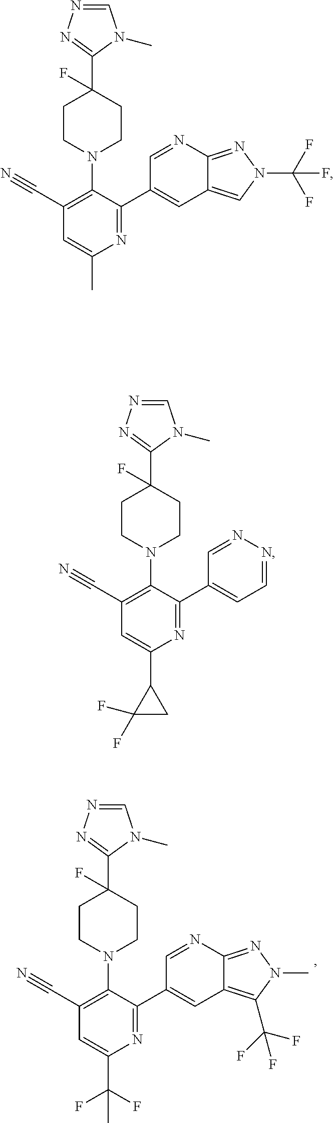 [SMILES: Cc1cc(C#N)c(N2CCC(F)(c3nncn3C)CC2)c(-c2cnc3nn(C(F)(F)F)cc3c2)n1 ; Cn1cnnc1C1(F)CCN(c2c(C#N)cc(C3CC3(F)F)nc2-c2ccnnc2)CC1 ; Cn1cnnc1C1(F)CCN(c2c(C#N)cc(C(C)(F)F)nc2-c2cnc3nn(C)c(C(F)(F)F)c3c2)CC1]

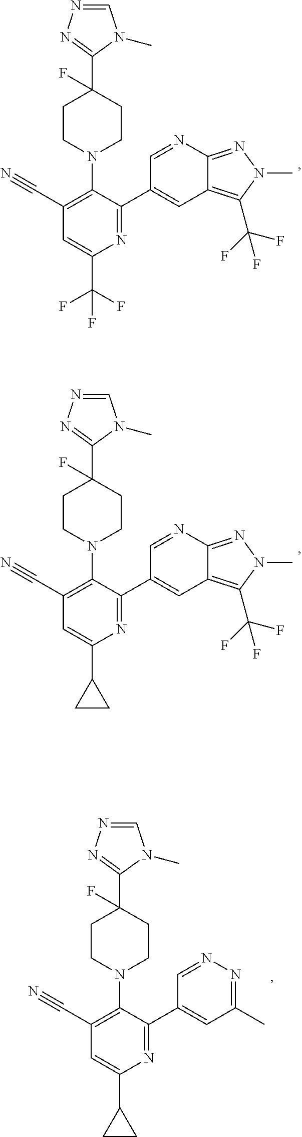 [SMILES: Cn1cnnc1C1(F)CCN(c2c(C#N)cc(C(F)(F)F)nc2-c2cnc3nn(C)c(C(F)(F)F)c3c2)CC1 ; Cn1cnnc1C1(F)CCN(c2c(C#N)cc(C3CC3)nc2-c2cnc3nn(C)c(C(F)(F)F)c3c2)CC1 ; Cc1cc(-c2nc(C3CC3)cc(C#N)c2N2CCC(F)(c3nncn3C)CC2)cnn1]

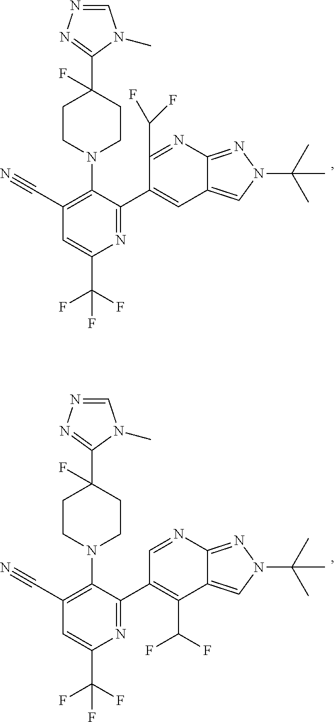 [SMILES: Cn1cnnc1C1(F)CCN(c2c(C#N)cc(C(F)(F)F)nc2-c2cc3cn(C(C)(C)C)nc3nc2C(F)F)CC1 ; Cn1cnnc1C1(F)CCN(c2c(C#N)cc(C(F)(F)F)nc2-c2cnc3nn(C(C)(C)C)cc3c2C(F)F)CC1]

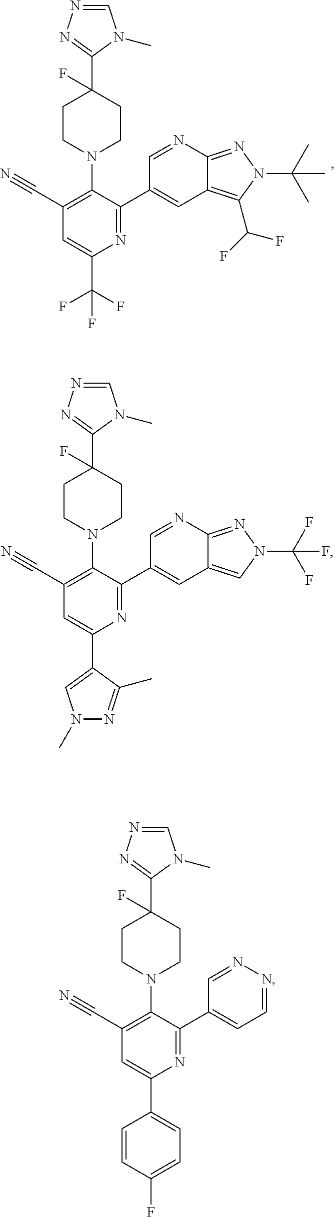 [SMILES: Cn1cnnc1C1(F)CCN(c2c(C#N)cc(C(F)(F)F)nc2-c2cnc3nn(C(C)(C)C)c(C(F)F)c3c2)CC1 ; Cc1nn(C)cc1-c1cc(C#N)c(N2CCC(F)(c3nncn3C)CC2)c(-c2cnc3nn(C(F)(F)F)cc3c2)n1 ; Cn1cnnc1C1(F)CCN(c2c(C#N)cc(-c3ccc(F)cc3)nc2-c2ccnnc2)CC1]

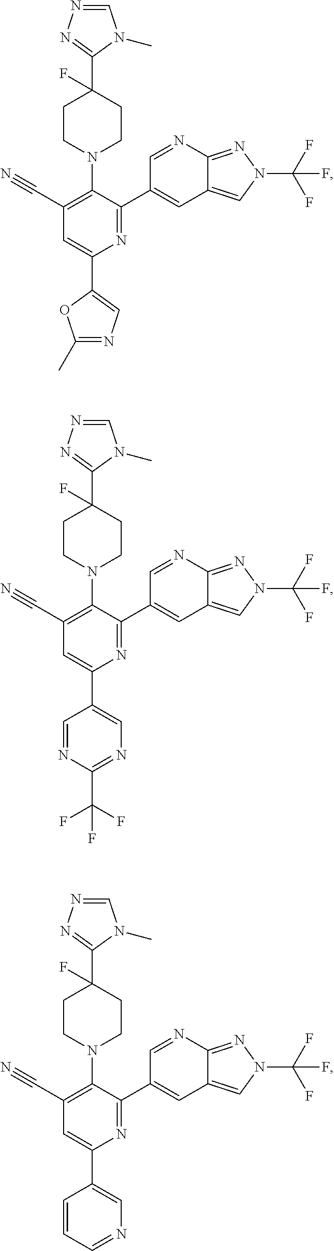 [SMILES: Cc1ncc(-c2cc(C#N)c(N3CCC(F)(c4nncn4C)CC3)c(-c3cnc4nn(C(F)(F)F)cc4c3)n2)o1 ; Cn1cnnc1C1(F)CCN(c2c(C#N)cc(-c3cnc(C(F)(F)F)nc3)nc2-c2cnc3nn(C(F)(F)F)cc3c2)CC1 ; Cn1cnnc1C1(F)CCN(c2c(C#N)cc(-c3cccnc3)nc2-c2cnc3nn(C(F)(F)F)cc3c2)CC1]

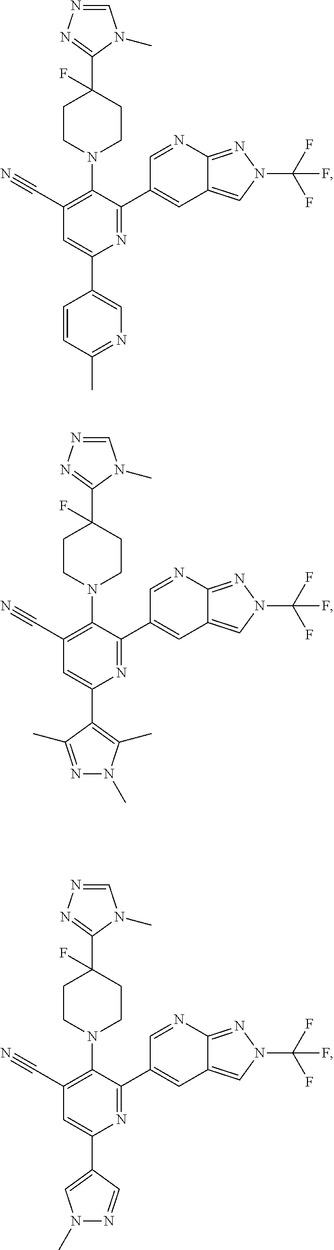 [SMILES: Cc1ccc(-c2cc(C#N)c(N3CCC(F)(c4nncn4C)CC3)c(-c3cnc4nn(C(F)(F)F)cc4c3)n2)cn1 ; Cc1nn(C)c(C)c1-c1cc(C#N)c(N2CCC(F)(c3nncn3C)CC2)c(-c2cnc3nn(C(F)(F)F)cc3c2)n1 ; Cn1cc(-c2cc(C#N)c(N3CCC(F)(c4nncn4C)CC3)c(-c3cnc4nn(C(F)(F)F)cc4c3)n2)cn1]

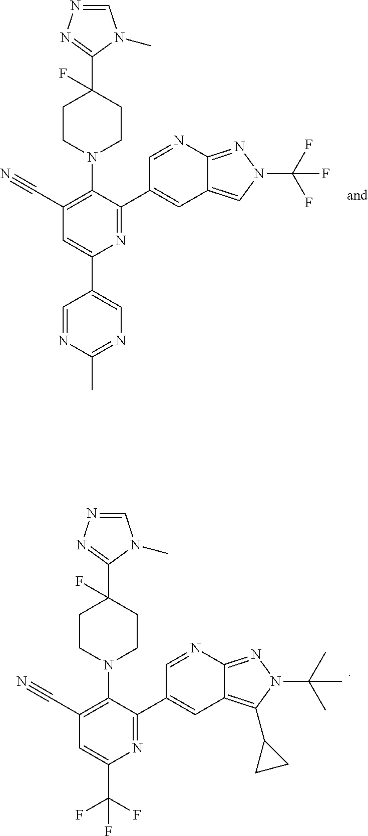 [SMILES: Cc1ncc(-c2cc(C#N)c(N3CCC(F)(c4nncn4C)CC3)c(-c3cnc4nn(C(F)(F)F)cc4c3)n2)cn1 ; Cn1cnnc1C1(F)CCN(c2c(C#N)cc(C(F)(F)F)nc2-c2cnc3nn(C(C)(C)C)c(C4CC4)c3c2)CC1]

Particularly preferred is the compound according to formula (I) selected from the group consisting of example 1, example 5, example 6, example 10, example 11, example 14, example 15, example 16, example 17, example 18, example 19, example 20, example 21, example 22, example 23, example 24, example 25 and example 26 as described hereinafter in EXAMPLES.

Particularly preferred is the compound according to formula (I) selected from the group consisting of example 1, example 6, example 18, example 19, example 22 and example 23, as described hereinafter in EXAMPLES.

Particularly preferred is the compound according to formula (I) selected from the group consisting of example 1, example 2, example 3, example 9, example 15, example 16, example 19, example 22 and example 26 as described hereinafter in EXAMPLES.

The present invention provides novel piperidinylpyridinylcarbonitrile derivatives of formula (I) that are surprisingly potent QPCT/L inhibitors.

Another aspect of the invention refers to compounds according to formula (I) as surprisingly having potent inhibition of QPCT/L in cells relevant for, but not limited to, lung diseases or cancer.

Another aspect of the invention refers to compounds according to formula (I) as surprisingly cellular potent QPCT/L inhibitors having appropriate membrane permeability, low in vitro efflux and low DDI perpetrator risk due to an appropriate CYP induction profile.

Another aspect of the invention refers to pharmaceutical compositions, containing at least one compound according to formula (I) optionally together with one or more inert carriers and/or diluents.

A further aspect of the present invention refers to compounds according to formula (I), for the use in the prevention and/or treatment of disorders associated with QPCT/L inhibition.

Another aspect of the invention refers to processes of manufacture of the compounds of the present invention.

Further aspects of the present invention will become apparent to the skilled artisan directly from the foregoing and following description and the examples.

### Used Terms and Definitions

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

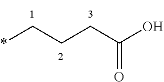 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

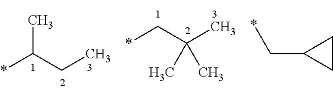

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “mono-heteroaryl ring” means a monocyclic aromatic ring system, containing one or more heteroatoms selected from N, O or S, consisting of 5 to 6 ring atoms.

The term “mono-heteroaryl ring” is intended to include all the possible isomeric forms. Thus, the term “mono-heteroaryl ring” includes the following exemplary structures (not depicted as radicals as each form is optionally attached through a covalent bond to any atom so long as appropriate valences are maintained):

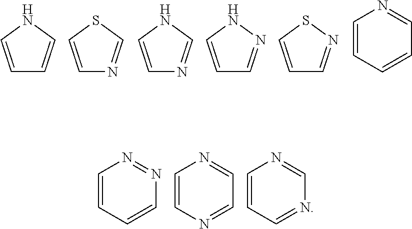

The term “fused bicyclic-heteroaryl ring” means a bicyclic aromatic ring system, containing one or more heteroatoms selected from N, O or S, consisting of 9 to 10 ring atoms. The term “fused bicyclic-heteroaryl ring” is intended to include all the possible isomeric forms. Thus, the term “bicyclic-heteroaryl ring” includes the following exemplary structures (not depicted as radicals as each form is optionally attached through a covalent bond to any atom so long as appropriate valences are maintained):

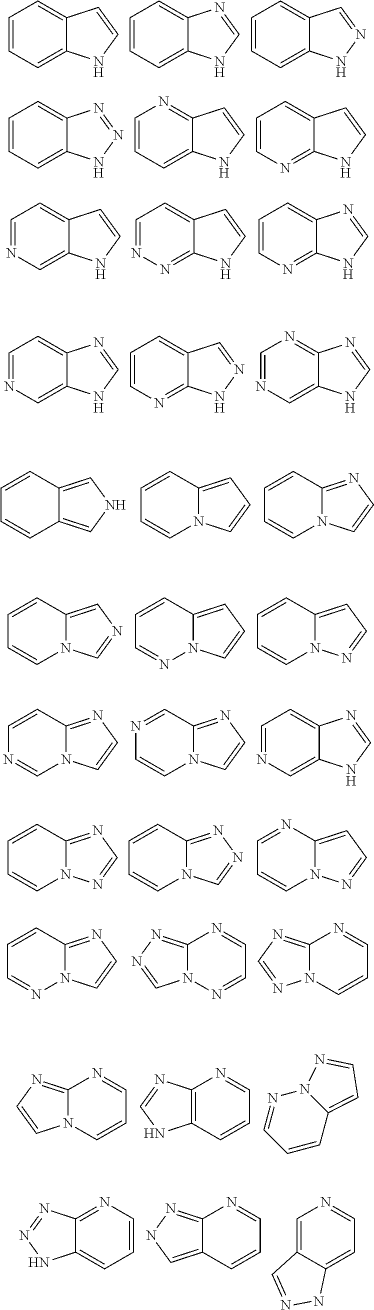

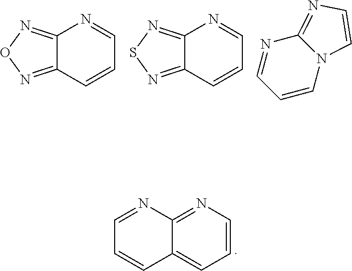

The term phenyl refers to the radical of the following ring:

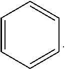

The term pyridyl refers to the radical of the following ring:

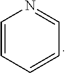

The term pyridazinyl refers to the radical of the following ring:

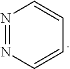

The term pyrimidyl refers to the radical of the following ring:

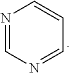

The term pyrazolyl refers to the radical of the following ring:

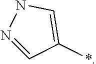

The term oxazolyl refers to the radical of the following ring:

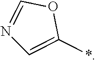

The term pvrazolopvridinvl refers to the radical of the following ring:

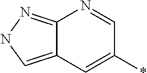

Many of the terms given above may be used repeatedly in the definition of a formula or group and in each case have one of the meanings given above, independently of one another.

### BIOLOGICAL ASSAYS

### Evaluation of Inhibitory Activity on QPCT and QPCTL

### Assay A: Biochemical QPCT and QPCTL Activity Assay

The activity of the compounds of the invention may be demonstrated using the following biochemical enzyme activity assay:

QPCT or QPCTL dependent conversion of N-terminal glutamine to pyroglutamate of CD47 was monitored via MALDI-TOF MS. Test compounds were dissolved in 100% DMSO and serially diluted into clear 1,536-well microtiter plates. Enzymatic reactions were set up in assay buffer containing 20 mM Tris pH 7.5, 0.1 mM TCEP, 0.01% BSA, and 0.001% Tween20. 2.5 L of 2× concentrated QPCTL (in-house) or QPCT (Origine #TP700028) enzyme in assay buffer (0.5 nM final concentration, columns 1-23) or plain assay buffer (columns 24) were added to each well. The plates were incubated for 10 min in a humidified incubator at 24° C. Subsequently, 2.5 μL of CD47 peptide substrate surrogate (¹⁹QLLFNKTKSVEFTFC³³) was added to each well (final concentration: 10 μM for QPCTL/20 μM for QPCT). The plates were mixed for 30 sec at 1,000 rpm and subsequently incubated for 40 min in a humidified incubator at 24° C. After incubation, the enzymatic reaction was stopped by adding 1 μL containing stable isotope labeled internal standard peptide ¹⁹[Pyr]LLFN(K)TKSVEFTFC³³ (final concentration 4.0 μM) as well as SEN177 (final concentration 10 μM). The plates were sealed with an adhesive foil, mixed for 30 s at 1,000 rpm and stored at room temperature until preparation of the MALDI target plates. MALDI target plates were prepared as described previously. 1 Mass spectra were acquired with a rapifleX MALDI-TOF/TOF instrument tracking the signals of the product (¹⁹[Pyr]LLFNKTKSVEFTFC³³, m/z 1,787.9037) as well as internal standard (¹⁹[Pyr]LLFN(K)TKSVEFTFC³³, m/z 1,795.9179) peptide. QPCT or QPCTL activity was monitored by calculating the ratio between product and internal standard signals followed by normalization to high (100% activity) and low (0% activity) controls. Determination of compound potencies was obtained by fitting the dose-response data to a four-parameter logistical equation.

TABLE 2
Biological data for compounds of the
invention as obtained in Assay A.

| | Inhibition | Inhibition |
|---|---|---|
| | of QPCTL: | of QPCT: |
| Example | IC₅₀ [nM] | IC₅₀ [nM] |
| 1 | 1 | 2 |
| 2 | 1 | 1 |
| 3 | 3 | 3 |
| 5 | 1 | 3 |
| 6 | 2 | 6 |
| 9 | 6 | 5 |
| 10 | 8 | 13 |
| 11 | 3 | 3 |
| 12 | 16 | 14 |
| 14 | 28 | 11 |
| 15 | 12 | 9 |
| 16 | 3 | 1 |
| 17 | 2 | 2 |
| 18 | 4 | 5 |
| 19 | 2 | 2 |
| 20 | 2 | 1 |
| 21 | 1 | 2 |
| 22 | 1 | 3 |
| 23 | 1 | 2 |
| 24 | 2 | 3 |
| 25 | 3 | 3 |
| 26 | 1 | 1 |

TABLE 3
Biological data for prior art compounds as obtained in Assay A.

| | | Inhibition | Inhibition |
|---|---|---|---|
| Prior art | | of QPCTL: | of QPCT: |
| Compound | Reference | IC₅₀ [nM] | IC₅₀ [nM] |
| Compound 14 | WO 2023/205173 | 1 | <1 |
| QP5038 | STTT 2023 | 2 | 5 |
| Compound | WO 2024/020517/ | 2 | 7 |
| (1)/QP5020 | STTT2023 | | |
| Compound 4 | WO 2024/020517 | 97 | 313 |
| Compound 11 | WO 2024/020517 | 1 | 6 |
| Compound 15 | WO 2024/020517 | 554 | 1083 |
| Compound 16 | WO 2024/020517 | 10 | 24 |
| Compound 19 | WO 2024/020517 | 117 | 106 |
| Compound 26 | WO 2024/020517 | 1 | 2 |
| Compound 33 | WO 2024/020517 | 1 | 2 |
| Compound 93 | WO 2024/020517 | 1 | 5 |
| Compound 94 | WO 2024/020517 | 1 | 3 |
| Compound 113 | WO 2024/020517 | 149 | 164 |
| Compound 125 | WO 2024/020517 | 159 | 446 |

### Assay B: SIRPα Signalling Assay (Using Either Raji or A549 Cells)

The activity of the compounds of the invention may be demonstrated using the following SIRPα signalling assay that measures SIRPα engagement induced by CD47 presented via cell-cell interaction. Two cell types are independently used: the Raji cell line (lymphoblast-like human cell line derived from B lymphocytes from a Burkitt's lymphoma patient in 1963) and A549 cells (adenocarcinoma human alveolar basal epithelial cells).

Test compounds were dissolved in 100% DMSO and serially diluted into a white 384-well microtiter cell culture plate (PerkinElmer #60076780 in case of Raji assay; PDL-coated plates Greiner #781945 in case of A549 assay). 5000 Raji cells (ATCC #CC86) or 5000 A549 cells (ATCC #CCL-185) in Assay Complete Cell Plating reagent 30 (DiscoverX 93-0563R30B) were added per well. The assay plate was incubated for 48 h at 37° C., 95% humidity and 5% CO₂. 15000 reporter cells (Jurkat PathHunter SIRPαV1, DiscoverX #93-1135C19) were added to each well, and the plate was incubated for 5 h at 37° C., 95% humidity and 5% CO₂. Bioassay reagent 1 of the PathHunter Bioassay detection kit (DiscoverX 93-0001) was added to each well of the plate using a multichannel pipette followed by a 15 min incubation at room temperature. Afterwards bioassay reagent 2 was added followed by 60 min incubation at room temperature (incubation in the dark).

The analysis of the data was performed using the luminescence signal generated by beta-galactosidase in the PathHunter reporter cell line. The luminescence measurement was done using a Pherastar Multi-Mode Reader. Dose-response curves & IC₅₀ data were calculated with 4-parameter sigmoidal dose response formula.

TABLE 4
Biological data for compounds of the
invention as obtained in Assay B.

| | Inhibition | Inhibition |
|---|---|---|
| | of SIRPα | of SIRPα |
| | signalling | signalling |
| | induced by | induced by |
| | Raji cells: | A549 cells: |
| Example | IC₅₀ [nM] | IC₅₀ [nM] |
| 1 | 107 | 15 |
| 2 | 20 | 14 |
| 3 | 10 | 7 |
| 5 | 31 | 6 |
| 6 | 226 | 82 |
| 9 | 83 | 14 |
| 10 | 160 | 49 |
| 11 | 167 | 37 |
| 12 | 950 | 145 |
| 14 | 117 | 44 |
| 15 | 3 | 1 |
| 16 | 11 | 17 |
| 17 | 69 | 22 |
| 18 | 262 | 82 |
| 19 | 139 | 25 |
| 20 | 618 | 110 |
| 21 | 78 | 24 |
| 22 | 130 | 17 |
| 23 | 169 | 43 |
| 24 | 73 | 27 |
| 25 | 181 | 52 |
| 26 | 4 | 11 |

TABLE 5
Biological data for prior art compounds as obtained in Assay B.

| | | Inhibition | Inhibition |
|---|---|---|---|
| | | of SIRPα | of SIRPα |
| | | signalling | signalling |
| | | induced by | induced by |
| Prior art | | Raji cells: | A549 cells: |
| Compound | Reference | IC₅₀ [nM] | IC₅₀ [nM] |
| Compound 14 | WO 2023/205173 | 4 | 4 |
| QP5038 | STTT 2023 | 21 | 6 |
| Compound | WO 2024/020517/ | 51 | 8 |
| (1)/QP5020 | STTT 2023 | | |
| Compound 4 | WO 2024/020517 | >10000 | 4501 |
| Compound 11 | WO 2024/020517 | 158 | 70 |
| Compound 15 | WO 2024/020517 | 5988 | 3153 |
| Compound 16 | WO 2024/020517 | 778 | 396 |
| Compound 19 | WO 2024/020517 | 2811 | 296 |
| Compound 26 | WO 2024/020517 | 452 | 208 |
| Compound 33 | WO 2024/020517 | 132 | 56 |
| Compound 93 | WO 2024/020517 | 1381 | 356 |
| Compound 94 | WO 2024/020517 | 2036 | 1298 |
| Compound 113 | WO 2024/020517 | >10000 | 4238 |
| Compound 125 | WO 2024/020517 | 4491 | >10000 |

### Evaluation of Permeability

### Assay C: Permeability in CACO-2 Cells

Caco-2 cells (1-2×10⁵ cells/1 cm² area) are seeded on filter inserts (Costar transwell polycarbonate or PET filters, 0.4 μm pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂, 4.17 mM NaHCO₃, 1.19 mM Na₂HPO₄×7H₂O, 0.41 mM NaH₂PO₄×H₂O, 15 mM HEPES, 20 mM glucose, 0.25% BSA, pH 7.2) to prepare the transport solutions (0.1-300 UM compound, final DMSO<=0.5%). The transport solution (TL) is applied to the apical or basolateral donor side for measuring A-B or B-A permeability (3 filter replicates), respectively. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

Efflux ratio (ER)=permeability B−A/permeability A−B

TABLE 7
Biological data for compounds of the
invention as obtained in Assay C.

| | Permeability A-B | Efflux |
|---|---|---|
| Example | [10⁻⁶ cm/s] | Ratio |
| 1 | 6.3 | 7.0 |
| 2 | 37.0 | 1.6 |
| 3 | 11.0 | 6.4 |
| 5 | 3.3 | 13.0 |
| 6 | 0.6 | 36.1 |
| 9 | 14.0 | 6.4 |
| 10 | 11.0 | 6.6 |
| 11 | 32.0 | 2.5 |
| 12 | 5.6 | 9.5 |
| 14 | 14.0 | 5.3 |
| 15 | 7.6 | 10.9 |
| 16 | 34.0 | 1.8 |
| 17 | 5.1 | 9.4 |
| 18 | 2.3 | 17.0 |
| 19 | 8.1 | 7.2 |
| 20 | 4.3 | 12.8 |
| 21 | 6.8 | 11.9 |
| 22 | 12.0 | 6.8 |
| 23 | 4.1 | 16.3 |
| 24 | 3.4 | 17.9 |
| 25 | 3.1 | 16.8 |
| 26 | 17.0 | 4.8 |

TABLE 8
Biological data for prior art compounds as obtained in Assay C.

| Prior art | | Permeability A-B | Efflux |
|---|---|---|---|
| Compound | Reference | [10⁻⁶ cm/s] | Ratio |
| Compound 14 | WO 2023/205173 | 0.7 | 22.2 |
| QP5038 | STTT 2023 | 61.0 | 0.8 |
| Compound | WO 2024/020517/ | 28.0 | 2.4 |
| (1)/QP5020 | STTT 2023 | | |
| Compound 4 | WO 2024/020517 | 0.3 | 28.1 |
| Compound 11 | WO 2024/020517 | 0.3 | 28.1 |
| Compound 15 | WO 2024/020517 | 11.0 | 5.4 |
| Compound 16 | WO 2024/020517 | <0.6 | >21.7 |
| Compound 19 | WO 2024/020517 | 19.0 | 3.4 |
| Compound 26 | WO 2024/020517 | <0.02 | >135 |
| Compound 33 | WO 2024/020517 | 0.2 | 45.3 |
| Compound 93 | WO 2024/020517 | <0.04 | >40 |
| Compound 94 | WO 2024/020517 | <1.4 | >0.8 |
| Compound 113 | WO 2024/020517 | <0.78 | >5 |
| Compound 125 | WO 2024/020517 | 0.6 | 30.4 |

### Evaluation of CYP3A4 Induction

### Assay D: CYP Induction Screening Assay in Primary Human Hepatocytes

Cryopreserved plateable human hepatocytes (single donor. BioIVT) were thawed and plated in Collagen-I coated 96-well-plates at a cell density of 0.07 million cells per well.

After a 6 h attachment period, the seeding medium was replaced by serum-free William's medium E supplemented with Matrigel (0.25 mg/ml) and allowed to recover overnight. 24 h post-seeding, serum-free Williams E medium containing the test compound at a final concentration of 10 M and a final DMSO content of 0.1% and 0.1% DMSO (solvent-treated control), respectively, was added to predefined wells. Exposure solutions were renewed after 24 h.

After 48 h of treatment in total, the effect of the test compounds on CYP3A4 mRNA expression was assessed using the QuantiGene Plex Gene Expression Assay. Hepatocytes were lysed and total RNA was extracted using the QuantiGene Sample Processing Kit according to the instructions of the manufacturer.

mRNA quantification was conducted using a customized QuantiGene Plex Panel to analyse CYP3A4 and the housekeeper genes RPL32, EIF4E2 and GUSB according to the instructions of the manufacturer and measured on a Luminex™ instrument. Signal was reported as median fluorescence intensity (MFI), which is proportional to the number of target RNA molecules present in the sample.

For calculation of CYP3A4 mRNA induction, the signal for CYP3A4 was normalized against the geometric mean of the signal obtained for the housekeeper genes for hepatocytes exposed to test compounds in relation to solvent-treated samples according to the following equation:

$$n ‐ fold induction = ( MFI CYP 3 A 4 ( treated ) / Xgeo MFI ( RPL 32 , EIF 4 E 2 , GUSB ) ) . ( MFICYP 3 A 4 ( Solvent control ) / Xgeo MFI ( RPL 32 , EIF 4 E 2 , GUSB ) )$$

TABLE 9
Biological data for compounds of the
invention as obtained in Assay D.

| | n-fold induction of |
|---|---|
| Example | CYP3A4 at 10 μM |
| 1 | 1.2 |
| 2 | 4.6 |
| 3 | 4.2 |
| 5 | 2.1 |
| 6 | 0.6 |
| 9 | 4.2 |
| 10 | 5.2 |
| 11 | 5.0 |
| 12 | 4.1 |
| 14 | |
| 15 | 14.2 |
| 16 | 6.0 |
| 17 | 3.0 |
| 18 | 1.3 |
| 19 | 1.1 |
| 20 | 0.5 |
| 21 | 0.7 |
| 22 | 0.5 |
| 23 | 1.1 |
| 24 | 1.1 |
| 25 | 0.8 |
| 26 | 7.9 |

TABLE 10
Biological data for prior art compounds as obtained in Assay D.

| Prior art | | n-fold induction of |
|---|---|---|
| Compound | Reference | CYP3A4 at 10 μM |
| Compound 14 | WO 2023/205173 | 1.9 |
| QP5038 | STTT 2023 | 58.5 |
| Compound | WO 2024/020517/ | 3.4 |
| (1)/QP5020 | STTT 2023 | |
| Compound 4 | WO 2024/020517 | 0.8 |
| Compound 11 | WO 2024/020517 | 1.1 |
| Compound 15 | WO 2024/020517 | 8.7 |
| Compound 16 | WO 2024/020517 | 1.5 |
| Compound 19 | WO 2024/020517 | 15.8 |
| Compound 26 | WO 2024/020517 | 1.1 |
| Compound 33 | WO 2024/020517 | 1.7 |
| Compound 93 | WO 2024/020517 | 0.8 |
| Compound 94 | WO 2024/020517 | <1 |
| Compound 113 | WO 2024/020517 | <1 |
| Compound 125 | WO 2024/020517 | 2.1 |

### Evaluation of Hepatic Stability (Mouse)

### Assay E: Stability in Murine Hepatocytes

The metabolic degradation of the test compound is assayed in a murine hepatocyte suspension.

Murine hepatocytes (typically cryopreserved) are incubated in an appropriate buffer system (e.g. Dulbecco's modified eagle medium plus 3.5 μg glucagon/500 mL, 2.5 mg insulin/500 mL and 3.75 mg/500 mL hydrocortison) containing 5% species serum.

Following a (typically) 30 min preincubation in an incubator (37° C., 10% CO2) 5 μl of test compound solution (80 μM; from 2 mM in DMSO stock solution diluted 1:25 with medium) are added into 395 μl hepatocyte suspension (cell density in the range 0.25-5 Mio cells/mL, typically 1 Mio cells/mL; final concentration of test compound 1 μM, final DMSO concentration 0.05%).

The cells are incubated for six hours (incubator, orbital shaker) and samples (25 μl) are taken at 0, 0.5, 1, 2, 4 and 6 hours. Samples are transferred into acetonitrile and pelleted by centrifugation (5 min). The supernatant is transferred to a new 96-deepwell plate, evaporated under nitrogen and resuspended.

Decline of parent compound is analyzed by HPLC-MS/MS

CLint is calculated as follows CL_INTRINSIC=Dose/AUC=(C0/CD)/(AUD+clast/k)×1000/60. C0: initial concentration in the incubation [μM], CD: cell density of vital cells [10e6 cells/mL], AUD: area under the data [μM×h], clast: concentration of last data point [μM], k: slope of the regression line for parent decline [h−1].

The calculated in vitro hepatic intrinsic clearance can be scaled up to the intrinsic in vivo hepatic Clearance and used to predict hepatic in vivo blood clearance (CL) by the use of a liver model (well stirred model).

$$CL\_INTRINSIC \_INVIVO [ m l / min / kg ] = ( CL\_INTRINSIC [ μ L / min / 10 e6cells ] \times hepatocellularity [ 10 e6cells / g liver ] \times liver factor [ g / kg bodyweight ] / 1000$$

$$CL [ ml / min / kg ] = CL\_INTRINSIC \_INVIVO [ ml / min / kg ] \times hepatic blood flow [ ml / min / kg ] / ( CL\_INTRINSIC \_INVIVO [ ml / min / kg ] + hepatic blood flow [ ml / min / kg ] )$$

$${Q}_{H} [ \% ] = C L [ ml / min / kg ] / hepatic blood flow [ ml / min / kg ] )$$

Hepatocellularity, mouse: 120×10e6 cells/g liver
Liver factor, mouse: 55 g/kg bodyweight
Blood flow, mouse: 90 ml/(min×kg)

TABLE 12
Biological data for compounds of the
invention as obtained in Assay E.

| | Mouse |
|---|---|
| | Hepatocyte |
| | Stability |
| Example | Q_{H} [%] |
| 1 | 49 |
| 2 | 49 |
| 3 | <12 |
| 5 | 16 |
| 6 | <12 |
| 9 | 34 |
| 10 | 53 |
| 11 | 32 |
| 12 | 21 |
| 14 | <12 |
| 15 | <12 |
| 16 | |
| 17 | 18 |
| 18 | 44 |
| 19 | <12 |
| 20 | <12 |
| 21 | 35 |
| 22 | 17 |
| 23 | <12 |
| 24 | <12 |
| 25 | 38 |
| 26 | 27 |

TABLE 13
Biological data for prior art compounds as obtained in Assay E.

| | | Mouse |
|---|---|---|
| | | Hepatocyte |
| Prior art | | Stability |
| Compound | Reference | Q_{H} [%] |
| Compound 14 | WO 2023/205173 | 33 |
| QP5038 | STTT 2023 | 97 |
| Compound | WO 2024/020517/ | 83 |
| (1)/QP5020 | STTT 2023 | |
| Compound 4 | WO 2024/020517 | 92 |
| Compound 11 | WO 2024/020517 | <12 |
| Compound 15 | WO 2024/020517 | 65 |
| Compound 16 | WO 2024/020517 | 61 |
| Compound 19 | WO 2024/020517 | 62 |
| Compound 26 | WO 2024/020517 | 19 |
| Compound 33 | WO 2024/020517 | 45 |
| Compound 93 | WO 2024/020517 | 26 |
| Compound 94 | WO 2024/020517 | 30 |
| Compound 113 | WO 2024/020517 | <12 |
| Compound 125 | WO 2024/020517 | 45 |

### Evaluation of Microsomal Clearance

### Microsomal Clearance:

The metabolic degradation of the test compound was assayed at 37° C. with pooled liver microsomes from various species. The final incubation volume of 60 μl per time point contains TRIS buffer pH 7.6 at room temperature (0.1 M), magnesium chloride (5 mM), microsomal protein (1 mg/mL for human and dog, 0.5 mg/mL for other species) and the test compound at a final concentration of 1 μM. Following a short preincubation period at 37° C., the reactions were initiated by addition of betanicotinamide adenine dinucleotide phosphate, reduced form (NADPH, 1 mM), and terminated by transferring an aliquot into solvent after different time points. After centrifugation (10000 g, 5 min), an aliquot of the supernatant was assayed by LC-MS/MS for the amount of parent compound. The half-life was determined by the slope of the semi-logarithmic plot of the concentration-time profile.

The intrinsic clearance (CL_INTRINSIC) is calculated by considering the amount of protein in the incubation:

$$CL\_INTRINSIC [ μl / min / mg protein ] = ( Ln 2 / ( half ‐ life [ min ] * protein content [ mg / ml ] ) * 1000$$

$$CL\_INTRINSIC \_INVIVO [ ml / min / kg ] = ( CL\_INTRINSIC [ μL / min / mg protein ] \times MPPGL [ mg protein / g liver ] \times liver factor [ g / kg bodyweight ] ) / 1000$$

$$Qh [ \% ] = CL [ ml / min / kg ] / hepatic blood flow [ ml / min / kg ]$$

Hepatocellularity, human: 120×10e6 cells/g liver
Liver factor, human: 25.7 g/kg bodyweight
Blood flow, human: 21 ml/(min×kg)

### Evaluation of Hepatocyte Clearance

Human Hepatocyte clearance

The metabolic degradation of a test compound is assayed in a human hepatocyte suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 5% human serum) to obtain a final cell density of 1.0×10⁶ cells/mL.

Following a 30 minutes preincubation in a cell culture incubator (37° C., 10% CO2), test compound solution is spiked into the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation. The supernatant is transferred to a 96-deepwell plate, and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T½).

In vitro intrinsic clearance (CLint) is calculated from in vitro T½ and scaled to whole liver using a hepatocellularity of 120×106 cells/g liver, a human liver per body weight of 25.7 g liver/kg as well as in vitro incubation parameters, applying the following equation:

$$CL\_INTRINSIC \_IN VIVO [ mL / min / kg ] = ( CL\_INTRINSIC [ μL / min / 106 cells ] \times hepatocellularity [ 106 cells / g liver ] \times liver factor [ g / kg body weight ] ) / 1000$$

Hepatic in vivo blood clearance (CL) is predicted according to the well-stirred liver model considering an average liver blood flow (QH) of 20.7 mL/min/kg:

$$CL [ mL / min / kg ] = CL\_INTRINSIC \_IN VIVO [ mL / min / kg ] \times hepatic blood flow [ mL / min / kg ] / ( CL\_INTRINSIC \_IN VIVO [ mL / min / kg ] + hepatic blood flow [ mL / min / kg ] )$$

Results are expressed as percentage of hepatic blood flow:

$${Q}_{H} [ \% ] = C L [ mL / min / kg ] / hepatic blood flow [ mL / min / kg ] )$$

### Evaluation of Plasma Protein Binding

Equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis.

Analyte concentrations are quantified in aliquots of samples by HPLC-MS/MS against calibration curves.

Percent bound is calculated using the formula:

$$\% bound = ( plasma concentration - buffer concentration / plasma concentration ) \times 100$$

### Evaluation of Solubility

Saturated solutions are prepared in well plates (format depends on robot) by adding an appropriate volume of selected aqueous media (typically in the range of 0.25-1.5 ml) into each well which contains a known quantity of solid drug substance (typically in the range 0.5-5.0 mg). The wells are shaken or stirred for a predefined time period (typically in a range of 2-24 h) and then filtered using appropriate filter membranes (typically PTFE-filters with 0.45 μm pore size). Filter absorption is avoided by discarding the first few drops of filtrate. The amount of dissolved drug substance is determined by UV spectroscopy. In addition, the pH of the aqueous saturated solution is measured using a glass-electrode pH meter.

### Evaluation of Metabolism in Human Hepatocytes In Vitro

The metabolic pathway of a test compound is investigated using primary human hepatocytes in suspension. After recovery from cryopreservation, human hepatocytes are incubated in Dulbecco's modified eagle medium containing 5% human serum and supplemented with 3.5 μg glucagon/500 ml, 2.5 mg insulin/500 ml and 3.75 mg/500 ml hydrocortisone.

Following a 30 min preincubation in a cell culture incubator (37° C., 10% CO₂), test compound solution is spiked into the hepatocyte suspension to obtain a final cell density of 1.0*10⁶ to 4.0*10⁶ cells/ml (depending on the metabolic turnover rate of the compound observed with primary human hepatocytes), a final test compound concentration of 10 μM, and a final DMSO concentration of 0.05%.

The cells are incubated for six hours in a cell culture incubator on a horizontal shaker, and samples are removed from the incubation after 0, 0.5, 1, 2, 4 or 6 hours, depending on the metabolic turnover rate. Samples are quenched with acetonitrile and pelleted by centrifugation. The supernatant is transferred to a 96-deepwell plate, evaporated under nitrogen and resuspended prior to bioanalysis by liquid chromatography-high resolution mass spectrometry for identification of putative metabolites.

The structures are assigned tentatively based on Fourier-Transform-MSⁿ data. Metabolites are reported as percentage of the parent in human hepatocyte incubation with a threshold of ≥4%.

### Evaluation of Pharmacokinetic Characteristics

The test compound is administered either intravenously or orally to the respective test species. Blood samples are taken at several time points post application of the test compound, anticoagulated and centrifuged.

The concentration of analytes—the administered compound and/or metabolites—are quantified in the plasma samples. PK parameters are calculated using non compartment methods. AUC and Cmax are normalized to a dose of 1 μmol/kg.

### Method of Treatment

The present invention is directed to compounds of general formula (I) which are useful in the prevention and/or treatment of a disease and/or condition associated with or modulated by QPCT/L activity, including but not limited to the treatment and/or prevention of cancer, fibrotic diseases, neurodegenerative diseases, atherosclerosis, infectious diseases, chronic kidney diseases.

The compounds of general formula (I) are useful for the prevention and/or treatment of:

(1) Pulmonary fibrotic diseases such as pneumonitis or interstitial pneumonitis associated with collagenosis, eg. lupus erythematodes, systemic scleroderma, rheumatoid arthritis, polymyositis and dermatomyositis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic organizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangiitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.

(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelofibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.

(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.

(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.

(5) Neurodegenerative disorders such as amyotrophic lateral sclerosis, multiple sclerosis, Parkinson's disease, Alzheimer's disease, Huntington's disease, multiple system atrophy, or prion diseases.

Accordingly, the present invention relates to a compound of general formula (I) or a pharmaceutically acceptable salt thereof for use as a medicament.

Furthermore, the present invention relates to the use of a compound of general formula (I) for the treatment and/or prevention of a disease and/or condition associated with or modulated by QPCT/L activity.

Furthermore, the present invention relates to the use of a compound of general formula (I) or a pharmaceutically acceptable salt thereof or a pharmaceutical composition thereof for the treatment and/or prevention of cancer, fibrotic diseases, neurodegenerative diseases, atherosclerosis, infectious diseases, chronic kidney diseases.

Furthermore, the present invention relates to the use of a compound of general formula (I) or a pharmaceutically acceptable salt thereof or a pharmaceutical composition thereof for the treatment and/or prevention of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic organizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangiitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.

(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelofibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.

(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.

(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.

(5) Neurodegenerative disorders such as amyotrophic lateral sclerosis, multiple sclerosis, Parkinson's disease, Alzheimer's disease, Huntington's disease, multiple system atrophy, or prion diseases.

In a further aspect the present invention relates to a compound of general formula (I) or a pharmaceutically acceptable salt thereof or a pharmaceutical composition thereof for use in the treatment and/or prevention of above-mentioned diseases and conditions.

In a further aspect the present invention relates to the use of a compound of general formula (I) or a pharmaceutically acceptable salt thereof or a pharmaceutical composition thereof for the preparation of a medicament for the treatment and/or prevention of above-mentioned diseases and conditions.

In a further aspect of the present invention the present invention relates to methods for the treatment or prevention of above-mentioned diseases and conditions, which method comprises the administration of an effective amount of a compound of general formula (I) or a pharmaceutically acceptable salt thereof or a pharmaceutical composition thereof to a human being.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with cancer, fibrotic diseases,

Alzheimer's diseases, atherosclerosis, infectious diseases, chronic kidney diseases and auto-immune disease.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of chemotherapy, targeted cancer therapy, cancer immunotherapy, irradiation, antifibrotic agents, anti-tussive agents, anti-inflammatory agents, anti-atopic dermatitis, and broncho dilators.

Chemotherapy is a type of cancer therapy that uses one or more chemical anti-cancer drugs, such as cytostatic or cytotoxic substances, cell proliferation inhibitors, anti-angiogenic substances and the like. Examples include folic acid (Leucovorin), 5-Fluorouracil, Irinotecan, Oxaliplatin, cis-platin Azacytidine, gemcitabine, alkylation agents, antimitotic agents, taxanes and further state-of-the-art or standard-of-care compounds.

Targeted therapy is a type of cancer treatment that uses drugs to target specific genes and proteins that help cancer cells survive and grow. Targeted therapy includes agents such as inhibitors of growth factors (e.g. platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor), tyrosine-kinases, KRAS, BRAF, BCR-ABL, mTOR, cyclin-dependent kinases, or MDM2.

Cancer immunotherapy is a type of therapy that uses substances to stimulate or suppress the immune system to help the body fight cancer. Cancer immunotherapy includes a therapeutic antibody, such as: anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab, or rituximab. Cancer immunotherapy also includes a therapeutic antibody which is a checkpoint inhibitor, such as an anti PD1, anti PD-L1 antibody or CTLA4 inhibitor. Examples include Atezolizumab, Avelumab, and Durvalumab, Ipilimumab, nivolumab, or pembrolizumab. Cancer immunotherapy also includes agents which target (inhibit) the CD47-SIRPα signaling axis, such as agents which bind to CD47 or SIRPα. Non-limiting examples include antibodies such as anti-CD47 antibodies and anti-SIRPα antibodies, and recombinant Fc-fusion proteins such as CD47-Fc and SIRPα-Fc. Cancer immunotherapy also includes STING-targeting agent, or T cell engagers, such as blinatumomab.

Antifibrotic agents are for example nintedanib, pirfenidone, phosphodiesterase-IV (PDE4) inhibitors such as roflumilast or specific PDE4b inhibitors like BI 1015550, autotaxin inhibitors such as GLPG-1690 or BBT-877; connective tissue growth factor (CTGF) blocking antibodies such as Pamrevlumab; B-cell activating factor receptor (BAFF-R) blocking antibodies such as Lanalumab, alpha-V/beta-6 blocking inhibitors such as BG-00011/STX-100, recombinant pentraxin-2 (PTX-2) such as PRM-151; c-Jun-N-terminal kinase (JNK) inhibitors such as CC-90001; galectin-3 inhibitors such as TD-139; G-protein coupled receptor 84 (GPR84) inhibitors; G-protein coupled receptor 84/G-protein coupled receptor 40 dual inhibitors such asPBI-4050, Rho Associated Coiled-Coil Containing Protein Kinase 2 (ROCK2) inhibitors such as KD-025, heat shock protein 47 (HSP47) small interfering RNA such as BMS-986263/ND-L02-s0201; Wnt pathway inhibitor such as SM-04646; LD4/PDE3/4 inhibitors such as Tipelukast; recombinant immuno-modulatory domains of histidyl tRNA synthetase (HARS) such as ATYR-1923, prostaglandin synthase inhibitors such as ZL-2102/SAR-191801; 15-hydroxy-eicosapentaenoic acid (15-HEPE e.g. DS-102); Lysyl Oxidase Like 2 (LOXL2) inhibitors such as PAT-1251, PXS-5382/PXS-5338; phosphoinositide 3-kinases (PI3K)/mammalian target of rapamycin (mTOR) dual inhibitors such as HEC-68498; calpain inhibitors such as BLD-2660; mitogen-activated protein kinase kinase kinase (MAP3K19) inhibitors such as MG-S-2525; chitinase inhibitors such as OATD-01,mitogen-activated protein kinase-activated protein kinase 2 (MAPKAPK2) inhibitors such as MMI-0100; transforming growth factor beta I (TGF-beta I) small interfering RNA such as TRKZSO/BNC-1021; or lysophosphatidic acid receptor antagonists such as BMS986278.

The dosage for the combination partners mentioned above is usually 1/5 of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions which may be affected or which are mediated by QPCT/L, in particular diseases or conditions as described hereinbefore and hereinafter.

In a further aspect this invention relates to a method for treating a disease or condition which can be influenced by the inhibition of QPCT/L in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of QPCT/L in a patient in need thereof.

In yet another aspect the present invention relates to a method for the treatment of a disease or condition mediated by QPCT/L activity in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis. Abbreviations are as defined in the Examples section.

Examples may be prepared as shown in Scheme I below.

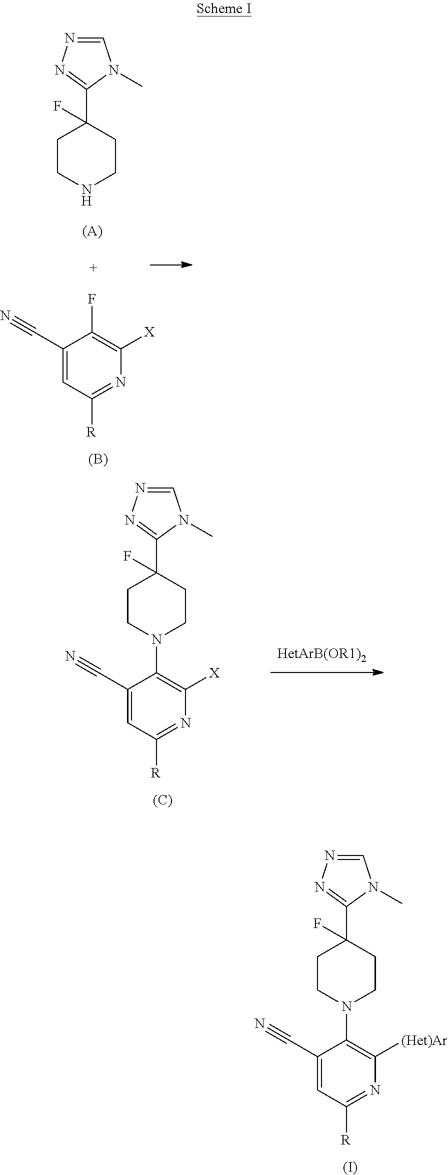

In scheme I, N-Methyl triazolyl piperidine (A) undergo a nucleophilic aromatic substitution with pyridinyl fluoride (X=Cl, Br) (B). The reaction can typically be run at ambient temperature or at elevated temperature (up to 110° C.) in the presence of a base (e.g. diisopropylethylamine). The intermediate (C) is then subjected to a Suzuki-cross coupling with a hetero-aryl boronic acid derivative in the presence of a suitable catalyst (e.g. Pd(dppf)Cl2) and a suitable base at elevated temperature (e.g. 100° C.) to afford compounds of general formula (I). The heteroaryl boronic acid derivatives are either commercially available or can be prepared by the corresponding heteroaryl bromides as described.

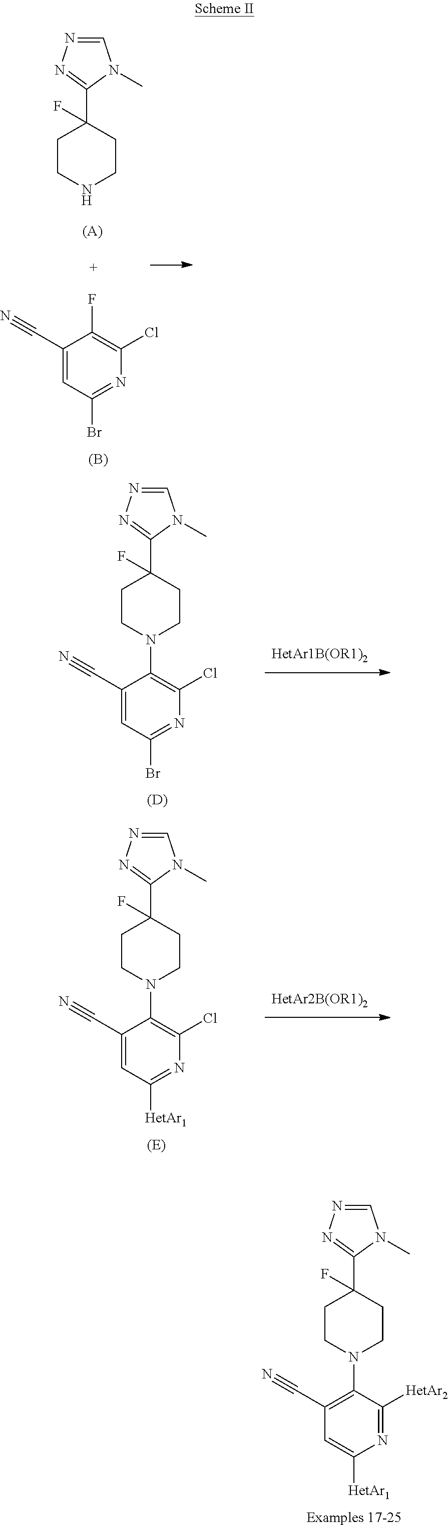

Compounds (D) can be prepared by reaction of piperidines (A) with fluoro-pyridonitriles (B) in the presence of a suitable base (e.g. di-isopropylethylamine). The reaction can typically be run at ambient temperature or at elevated temperature (up to 110° C.) in the presence of a base (e.g. diisopropylethylamine). The intermediate (D) is then subjected to a Suzuki-cross coupling with a hetero-aryl boronic acid derivative (HetAr1B(OR1)2) in the presence of a suitable catalyst (e.g. Pd(dtbpf)Cl₂) and a suitable base at elevated temperature (e.g. 100° C.) to afford compounds of general formula (E). Examples 17-25 can be obtained by Suzuki-cross coupling with another hero-aryl boronic acid derivative (HetAr2B(OR¹)₂) derivative in the presence of a suitable catalyst (e.g. Pd(dppf)Cl2) and a suitable base at elevated temperature (e.g. 100° C.) to afford examples 17-25.

Intermediates (A) may be prepared as shown in Scheme III below:

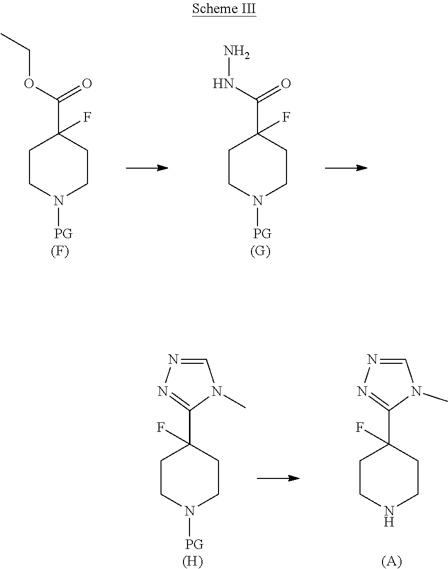

Compounds of formula (A) can be prepared from the corresponding piperidinyl esters (F) equipped with a suitable protecting group (PG, e.g. BOC) by treatment with a suitable hydrazine source (e.g. N₂H₄*H₂O) at elevated temperature (e.g. 50° C.). The obtained hydrazide (G) is then activated with DMF/DMA at elevated temperature (e.g. 50° C.) and subsequently treated with methyl amine at elevated temperature (e.g. 90° C.) to yield the triazole derivative (H). Compounds of formula (A) can be obtained by cleaving the protecting group under suitable conditions (e.g. TFA).

Intermediates (B) may be prepared as shown in Scheme IV below:

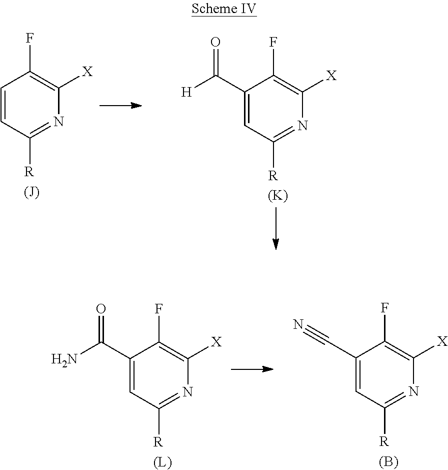

Deprotonation of commercially available pyridines (J) at low temperature (e.g. −65° C.) and quenching with DMF yields the corresponding aldehydes (K), aldehyde (K) can be transformed into the amide (L) using a suitable reagent, e.g. phenyltrimethylammonium tribromide, at ambient temperature. Compounds of formula (B) are subsequently obtained by treatment of (L) with a suitable dehydrating agent, e.g. Burgess reagent at ambient temperature.

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

### Abbreviations

ACN acetonitrile
Aq. aqueous
brine saturated aqueous NaCl solution
® C. degree celsius
CyH/CH cyclohexane
CO₂ carbon dioxide
conc. concentrated
Cs₂CO₃ cesium carbonate
DCM dichloromethane
DIPA N,N-diisopropylamine
DIPEA N,N-diisopropylethylamine
DMF N,N-dimethylformamide
DMSO dimethyl sulfoxide
ESI-MS Electrospray ionisation mass spectrometry
EtOAc/EA ethyl acetate
EtOH ethanol
ex example
eq equivalent
FA formic acid
h hour
H₂O water
HCl hydrochloric acid
HPLC high performance liquid chromatography
Int. intermediate
K₂CO₃ potassium carbonate
K₃PO₄ tripotassium phosphate
KOAc potassium acetate
KOH potassium hydroxide
L liter
LDA lithium diisopropylamide
LiOH lithium hydroxide
M Molar (mol/L)
MeOH methanol
MeTHF methyl tetrahydrofuran
MgSO₄ magnesium sulfate
min minute
mL milliliter
MTBE Methyl-tert-butylether
μL microliter
N₂ nitrogen
n-BuLi n-Butyllithium
NBS N-Bromosuccinimide
NCS N-Chlorosuccinimide
Na₂CO₃ sodium carbonate
NaHCO₃ sodium bicarbonate
NH₃ ammonia
NH₄Cl ammonium chloride
NaOH sodium hydroxide
Na₂SO₄ sodium sulfate
PdCl₂(PPh₃)₂ Bis(triphenylphosphine)palladium(II) dichloride
Pd(dppf)Cl₂ 1,1′-Bis(diphenylphosphino)ferrocene palladium(II)dichloride
Pd(dtbpf)Cl₂ 1,1′-Bis(di-tert-butylphosphino)ferrocene palladium(II)dichloride
Pd(PPh₃)₄ Tetrakis(triphenylphosphine)palladium(0)
PE petroleum ether
Prep. preparative
RP reversed phase
RT/rt room temperature (about 20° C.)
sat. saturated
SFC Supercritical Fluid Chromatography
SiO₂ silica
TEA triethylamine
TFA trifluoroacetic acid
TFAA trifluoroacetic anhydride
THF tetrahydrofuran
Xphos Pd G3 (2-Dicyclohexylphosphino-2′,4′,6′-triisopropyl-1,1′-biphenyl)[2-(2′-amino-1,1′-biphenyl)]palladium(II) methanesulfonate

### PREPARATION OF INTERMEDIATES

### Synthesis of Intermediate I.1

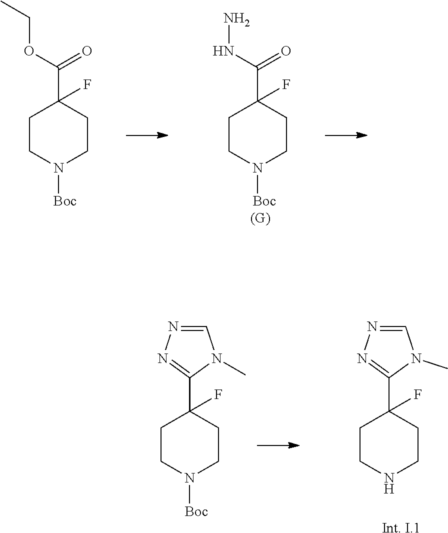

### tert-Butyl 4-fluoro-4-(hydrazinecarbonyl) piperidine-1-carboxylate

1-tert-Butyl 4-ethyl 4-fluoropiperidine-1,4-dicarboxylate (160 g, 0.58 mol) is suspended in ethanol (640 mL) in a round-bottom flask. Hydrazine hydrate (70.6 mL, 1.16 mol) is added to the mixture at ambient temperature. The reaction mixture is heated to 50° C. and stirred for 12 h. After cooling to ambient temperature, the mixture is concentrated under reduced pressure to yield tert-butyl 4-fluoro-4-(hydrazinecarbonyl) piperidine-1-carboxylate in 80% purity.

C₁₁H₂₀FN₃O₃ (M=261.3 g/mol)

ESI-MS: 284 [M+Na]⁺

Rt (HPLC): 0.62 min (method A)

### tert-Butyl 4-fluoro-4-(4-methyl-4H-1,2,4-triazol-3-yl)piperidine-1-carboxylate

tert-Butyl 4-fluoro-4-(hydrazinecarbonyl) piperidine-1-carboxylate (135 g, 0.413 mol, 80% purity) is mixed with dioxane (945 mL) in a round-bottom-flask. N,N-Dimethylformamid-dimethylacetal (137 mL, 1.03 mol) is added to the mixture at ambient temperature. The reaction mixture is heated to 50° C. and stirred for 1 h. A solution of methylamine (299 g, 30% in EtOH, 2.89 mol) and acetic acid (165 mL, 2.89 mol) are added into the mixture. The resulting reaction mixture is heated to 90° C. and stirred for 11 h. After cooling to ambient temperature, the mixture is concentrated under reduced pressure. The residue is purified by column chromatography (SiO₂, PE/EtOAc gradient 20:1 to 0:1) to obtain tert-butyl 4-fluoro-4-(4-methyl-4H-1,2,4-triazol-3-yl)piperidine-1-carboxylate.

C₁₃H₂₁FN₄O₂ (M=284.3 g/mol)

ESI-MS: 285 [M+H]⁺

Rt (HPLC): 0.77 min (method A)

### Intermediate I.1: 4-fluoro-4-(4-methyl-4H-1,2,4-triazol-3-yl)piperidine

tert-Butyl 4-fluoro-4-(4-methyl-4H-1,2,4-triazol-3-yl)piperidine-1-carboxylate (90 g, 0.32 mol) is combined with methanol (90 mL) in a round-bottom flask. A solution of HCl (4 m in MeOH, 450 mL, 1.8 mol) is added slowly at ambient temperature. The resulting reaction mixture is stirred at ambient temperature for 12 h. The desired product is collected by filtration, washed with methanol, and dried to yield 4-fluoro-4-(4-methyl-4H-1,2,4-triazol-3-yl) piperidine hydrochloride salt.

The hydrochloride salt (13.5 g) is added to a solution of ammonia in methanol (7 M, 150 mL) and purified by column chromatography (Biotage SNAP Cartridge KP-NH 110 g, gradient DCM/MeOH 4:1 to 7:3) to afford the title compound.

C₈H₁₃FN₄ (M=184.2 g/mol)

ESI-MS: 185 [M+H]⁺

Rt (HPLC): 0.20 min (method B)

### Synthesis of Intermediate II.1 and II.2

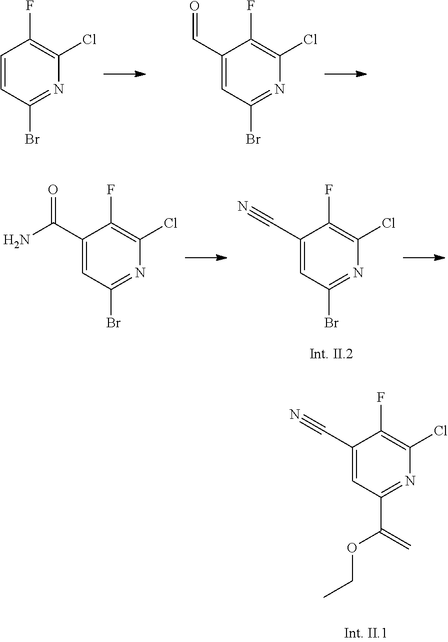

### 6-Bromo-2-chloro-3-fluoropyridine-4-carbaldehyde

Under an argon atmosphere, 6-bromo-2-chloro-3-fluoropyridine (6.80 g, 30.7 mmol) is added to THF (30 mL), and the resulting mixture is cooled to −75° C. A solution of lithium diisopropylamide (1 M in THF, 30.7 mL, 30.7 mmol) is added dropwise, and the mixture is stirred for 1 h at −75° C. DMF (2.83 mL, 36.8 mmol) is added dropwise. The mixture is stirred for additional 2 h at −78° C. The reaction is quenched by addition of acetic acid (2.64 mL) and diluted with water/brine 1/1 and ethyl acetate and allowed to warm to ambient temperature. The organic phase is separated, dried over Na₂SO₄, and concentrated. The residue is purified by column chromatography (SiO₂, CyH/EtOAc gradient 1:0 to 1:1) to yield 6-bromo-2-chloro-3-fluoropyridine-4-carbaldehyde.

C₆H₂BrClFNO (M=238.4 g/mol)

ESI-MS: mass not detected

Rt (HPLC): 0.44 min (method B)

### 6-Bromo-2-chloro-3-fluoropyridine-4-carboxamide

Ammonium acetate (12.0 g, 15.5 mmol) and 6-Bromo-2-chloro-3-fluoropyridine-4-carbaldehyde (3.70 g, 15.5 mmol) are mixed, and acetonitrile (50 mL) is added. Phenyltrimethylammonium tribromide (12.0 g, 31.0 mmol) is added in small portions, and the resulting reaction mixture is stirred for 16 h at ambient temperature. The mixture is filtered, and the residue is washed with acetonitrile, purified by column chromatography (dry load, SiO₂, CyH/EtOAc gradient 1:0 to 1:1) to yield the desired product.

C₆H₃BrClFN₂O (M=253.5 g/mol)

ESI-MS: 251/253 [M−H]⁻

Rt (HPLC): 0.41 min (method B)

### Intermediate II.2: 6-Bromo-2-chloro-3-fluoropyridine-4-carbonitrile

6-Bromo-2-chloro-3-fluoropyridine-4-carboxamide (510 mg, 2.01 mmol) is suspended in dichloromethane (10 mL), and Burgess reagent (CAS: 29684-56-8, 742 mg, 3.02 mmol) is added at ambient temperature. The resulting reaction mixture is stirred for 16 h and then directly purified by column chromatography (SiO₂, CyH/EtOAc gradient 1:0 to 9:1) to yield the title compound.

C₆HBrClFN₂ (M=235.4 g/mol)

ESI-MS: no mass detected

Rt (HPLC): 0.61 min (method B)

¹H NMR (400 MHZ, DMSO-d₆) δ ppm 8.45 (d, J=3.8 Hz, 1H).

### Intermediate II.1:2-Chloro-6-(1-ethoxyethenyl)-3-fluoropyridine-4-carbonitrile

Under an argon atmosphere, Int. II.2 (50.0 mg, 0.21 mmol) and tributyl(1-ethoxyethenyl) stannane (87.9 μL, 0.23 mmol) are suspended in 1.4-dioxane (0.5 mL). Then, [1,1′-Bis-(diphenylphosphino)-ferrocen]-dichloro-palladium (II) (Pd(dppf)Cl₂, CAS: 72287-26-4) (15.5 mg, 0.02 mmol) is added, and the mixture is further degassed for 5 min. The reaction mixture is heated at 70° C. for 10 h. The mixture is concentrated and the residue is purified by column chromatography (dry load, SiO₂, CyH/EtOAc gradient 1:0 to 9:1) to yield the title compound.

C₁₀H₈ClFN₂O (M=226.6 g/mol)

ESI-MS: no mass detected

Rt (HPLC): 0.80 min (method B)

¹H NMR (400 MHz, DMSO-d₆) δ ppm 8.08 (d, J=4.1 Hz, 1H), 5.31 (d, J=2.3 Hz, 1H), 4.62 (d, J=2.3 Hz, 1H), 3.97 (q, J=6.8 Hz, 2H), 1.38 (t, J=7.0 Hz, 3H).

### Synthesis of Intermediate II.5

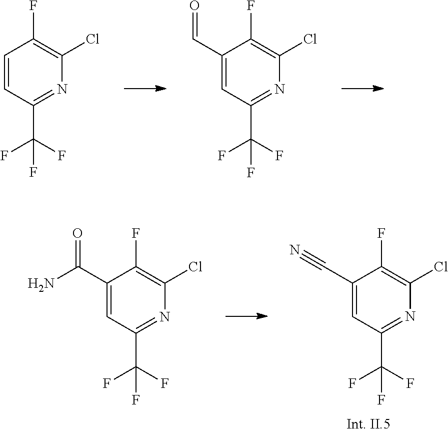 [SMILES: Fc1ccc(C(F)(F)F)nc1Cl ; O=Cc1cc(C(F)(F)F)nc(Cl)c1F ; NC(=O)c1cc(C(F)(F)F)nc(Cl)c1F ; N#Cc1cc(C(F)(F)F)nc(Cl)c1F]

### 2-Chloro-3-fluoro-6-(trifluoromethyl)pyridine-4-carbaldehyde

Under an argon atmosphere, 2-chloro-3-fluoro-6-(trifluoromethyl)pyridine (14.2 g, 69.7 mmol) is added to THF (330 mL), and the resulting mixture is cooled to −75° C. A solution of lithium diisopropylamide (1 M in THF, 77.0 mL, 77.0 mmol) is added dropwise in a period of 90 min, and the mixture is stirred for 60 min at −75° C. DMF (6.44 mL, 83.7 mmol) is added dropwise. The mixture is stirred for additional 30 min at −75° C. The reaction is quenched by addition of a half concentrated acetic acid (40 mL) and diluted with water and EtOAc. The organic phase is separated, washed with brine, dried, and concentrated. The residue is purified by column chromatography (SiO₂, CyH/EtOAc gradient 100:0 to 85:15) to yield the desired compound.

C₇H₂ClF₄NO (M=227.5 g/mol)

EI-MS: 227 M*+

Rt (HPLC): 0.48 min (method B)

### 2-Chloro-3-fluoro-6-(trifluoromethyl)pyridine-4-carboxamide

Ammonium acetate (47.1 g, 611 mmol) and 2-chloro-3-fluoro-6-(trifluoromethyl)pyridine-4-carbaldehyde (13.9 g, 61.1 mmol) are mixed, and acetonitrile (300 mL) is added. Phenyltrimethylammonium tribromide (47.4 g, 122 mmol) is added in small portions, and the resulting reaction mixture is stirred at ambient temperature for 16 h. The mixture is filtered through a pad of silica, and the residue is washed with acetonitrile, purified by column chromatography (SiO₂, CyH/EtOAc gradient 1:0 to 7:3) to yield the desired product.

C₇H₃ClF₄N₂O (M=242.6 g/mol)

ESI-MS: 241 [M−H]⁻

Rt (HPLC): 0.47 min (method B)

### Intermediate II.5: 2-chloro-3-fluoro-6-(trifluoromethyl)pyridine-4-carbonitrile

The product of the previous step, 2-chloro-3-fluoro-6-(trifluoromethyl)pyridine-4-carboxamide (5.10 g, 21.0 mmol) is suspended in dichloromethane (400 mL), and Burgess reagent (CAS: 29684-56-8, 8.75 g, 35.6 mmol) is added at ambient temperature. The resulting reaction mixture is stirred for 40 h and then directly purified by column chromatography (SiO₂, CyH/EtOAc gradient 100:0 to 95:5) to yield the title compound.

C7HClF₄N₂ (M=224.5 g/mol)

ESI-MS: 225 [M+H]⁺

Rt (HPLC): 0.57 min (method D)

### Synthesis of Intermediate II.6

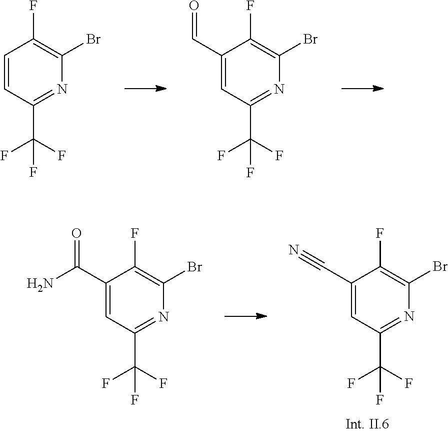 [SMILES: Fc1ccc(C(F)(F)F)nc1Br ; O=Cc1cc(C(F)(F)F)nc(Br)c1F ; NC(=O)c1cc(C(F)(F)F)nc(Br)c1F ; N#Cc1cc(C(F)(F)F)nc(Br)c1F]

### 2-Bromo-3-fluoro-6-(trifluoromethyl)pyridine-4-carbaldehyde

Under an argon atmosphere, 2-bromo-3-fluoro-6-(trifluoromethyl)pyridine (3.08 g, 12.6 mmol) is added to THF (75 mL), and the resulting mixture is cooled to −70° C. A solution of lithium diisopropylamide (1 M in THF, 13.9 mL, 13.9 mmol) is added dropwise, and the mixture is stirred for 90 min at −70° C. DMF (1.17 mL, 15.1 mmol) is added dropwise. The mixture is stirred for additional 30 min at −70° C. The reaction is quenched by addition of a half concentrated acetic acid (800 μL) and diluted with water and EtOAc. The organic phase is separated, dried over MgSO₄, and concentrated. The residue is purified by column chromatography (SiO₂, CyH/EtOAc gradient 1:0 to 4:1) to yield 2-bromo-3-fluoro-6-(trifluoromethyl)pyridine-4-carbaldehyde.

C₇H₂BrF₄NO (M=272.0 g/mol)

ESI-MS: no mass detected

Rt (HPLC): 0.51 min (method B)

¹H NMR (400 MHZ, DMSO-d₆) δ ppm 10.19 (s, 1H), 8.26 (d, J=4.3 Hz, 1H).

### 2-Bromo-3-fluoro-6-(trifluoromethyl)pyridine-4-carboxamide

Ammonium acetate (5.53 g, 71.8 mmol) and 2-bromo-3-fluoro-6-(trifluoromethyl)pyridine-4-carbaldehyde (2.17 g, 7.18 mmol) are mixed, and acetonitrile (44 mL) is added. Phenyltrimethylammonium tribromide (5.57 g, 14.4 mmol) is added in small portions, and the resulting reaction mixture is stirred for 72 h at ambient temperature. The mixture is filtered, and the residue is washed with acetonitrile, purified by column chromatography (dry load with Celite®, SiO₂, CyH/EtOAc gradient 1:0 to 7:3) to yield the desired product.

C₇H₃BrF₄N₂O (M=287.0 g/mol)

ESI-MS: 285/287 [M−H]⁻

Rt (HPLC): 0.50 min (method B)

### Intermediate II.6: 2-bromo-3-fluoro-6-(trifluoromethyl)pyridine-4-carbonitrile

Product of the previous step, 2-bromo-3-fluoro-6-(trifluoromethyl)pyridine-4-carboxamide (975 mg, 3.40 mmol) is suspended in dichloromethane (80 mL), and Burgess reagent (CAS: 29684-56-8, 1.25 g, 5.10 mmol) is added at ambient temperature. The resulting reaction mixture is stirred for 40 h and then directly purified by column chromatography (dry load, SiO₂, CyH/EtOAc gradient 1:0 to 9:1) to yield the title compound.

C₇HBrF₄N₂ (M=268.9 g/mol)

ESI-MS: 268/270 [M+H]⁺

Rt (HPLC): 0.59 min (method D)

### Intermediate III.1: 6-bromo-2-chloro-3-[4-fluoro-4-(4-methyl-4H-1,2,4-triazol-3-yl)piperidin-1-yl]pyridine-4-carbonitrile

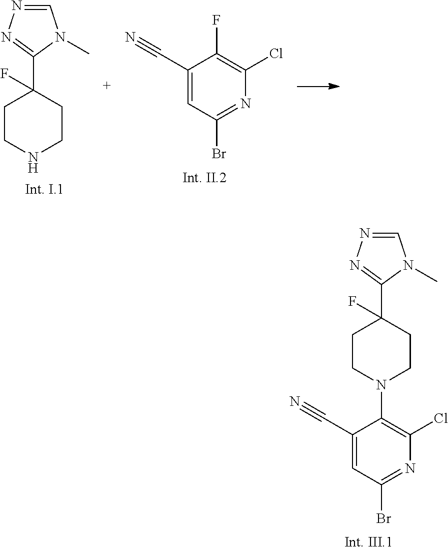

Int. II.2 (1.00 g, 4.25 mmol) is suspended in DMSO (4.0 mL) and DIPEA (1.47 mL, 8.50 mmol). At 15° C., Int. I.1 (900 mg, 4.89 mmol) is added, and the resulting mixture is stirred at 15° C. for 2 h. The mixture is diluted with ACN/water and purified by preparative HPLC (Sunfire C18, acetonitrile/water gradient containing 0.1% TFA) to afford the desired compound.

C₁₄H₁₃BrClFN₆ (M=399.6 g/mol)

ESI-MS: 399/401 [M+H]⁺

Rt (HPLC): 0.75 min (method E)

### Intermediates Synthesized Analogous to the Procedure Described for Int. III.1

| | | | | Molecular |
|---|---|---|---|---|
| | | | | Formula |
| | | | | (MW) |
| | | | | ESI-MS |
| | | | Deviation from | HPLC |
| | Starting | | general | retention time |
| Int. | material | Structure | procedure | (method) |
| III.2 | Int. I.1 + 2-chloro-3- fluoro-6- methylpyridine- 4-carbonitrile | 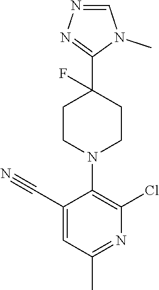 [SMILES: Cc1cc(C#N)c(N2CCC(F)(c3nncn3C)CC2)c(Cl)n1] | 10° C.→rt 18 h @ rt trituration with water and purification by SiO₂ chromatography (DCM/MeOH 100:0 to 88:12) | C₁₅H₁₆ClFN₆ (M = 334.8 g/mol) ESI-MS: 335 [M + H]⁺ Rt (HPLC): 0.80 min (method C) |
| III.3 | Int. I.1 + Int. II.1 | 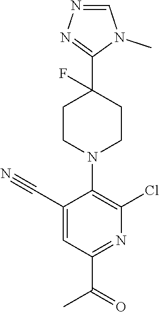 [SMILES: CC(=O)c1cc(C#N)c(N2CCC(F)(c3nncn3C)CC2)c(Cl)n1] | 1.5 equiv. of Int. I.1 16 h @ 15° C.; crude product treated with 4M HCl in 1.4- dioxane for 3 h at ambient temperature, concentrated, and basified with aq. K₂CO₃ | C₁₆H₁₆ClFN₆O (M = 362.8 g/mol) ESI-MS: 363 [M + H]⁺ Rt (HPLC): 0.66 min (method E) |
| III.4 | Int. I.1 + Int. II.5 | 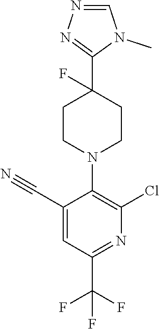 [SMILES: Cn1cnnc1C1(F)CCN(c2c(C#N)cc(C(F)(F)F)nc2Cl)CC1] | 15 min @ rt purification by preparative HPLC (XBridge C18, acetonitrile/water gradient containing 0.1% NH₃) | C₁₅H₁₃ClF₄N₆ (M = 388.8 g/mol) ESI-MS: 389/391 [M + H]⁺ Rt (HPLC): 0.59 min (method B) |
| III.5 | Int. I.1 + Int. II.6 | 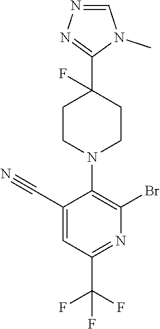 [SMILES: Cn1cnnc1C1(F)CCN(c2c(C#N)cc(C(F)(F)F)nc2Br)CC1] | 1.5 h @ 15° C. purification by preparative HPLC (XBridge C18, acetonitrile/water gradient containing 0.1% NH₃) | C₁₅H₁₃BrF₄N₆ (M = 433.2 g/mol) ESI-MS: 433/435 [M + H]⁺ Rt (HPLC): 0.54 min (method D) |

### Intermediate III.6: 2-chloro-6-cyclopropyl-3-[4-fluoro-4-(4-methyl-4H-1,2,4-triazol-3-yl)piperidin-1-yl]pyridine-4-carbonitrile

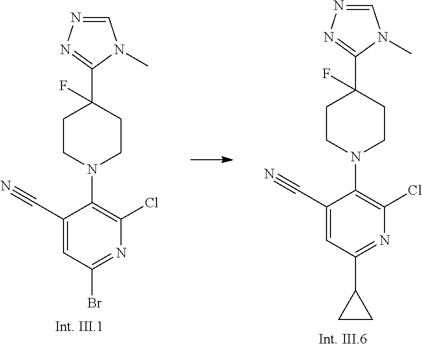 [SMILES: Cn1cnnc1C1(F)CCN(c2c(C#N)cc(Br)nc2Cl)CC1 ; Cn1cnnc1C1(F)CCN(c2c(C#N)cc(C3CC3)nc2Cl)CC1]

Under an argon atmosphere, Int. III.1 (9.90 g, 21.1 mmol) and cyclopropyl boronic acid (9.04 g, 105.3 mmol) are suspended in 1.4-dioxane (300 mL). A solution of K₂CO₃ (aq. 2 M, 31.6 mL, 63.2 mmol) is added, and the resulting mixture is degassed for 10 min by passing an argon stream through the mixture. [1,1′-Bis-(diphenylphosphino)-ferrocen]-dichloro-palladium(II) (Pd(dppf)Cl₂) (1.54 g, 2.11 mmol) is added, and the mixture is degassed again for 5 min. The mixture is then heated to 70° C. for 25 h. After cooling to ambient temperature, the mixture is loaded onto Extrelut and purified by column chromatography (ethyl acetate/methanol gradient 100:0 to 90:10) to yield a mixture of starting material and product. Upon concentration to 30 mL, the precipitate formed is collected and dried to yield the desired product.

C₁₇H₁₈ClFN₆ (M=360.8 g/mol)

ESI-MS: 361 [M+H]⁺

Rt (HPLC): 0.56 min (method D)

### Intermediate III.7: 2-chloro-6-(2,2-difluorocyclopropyl)-3-[4-fluoro-4-(4-methyl-4H-1,2,4-triazol-3-yl)piperidin-1-yl]pyridine-4-carbonitrile

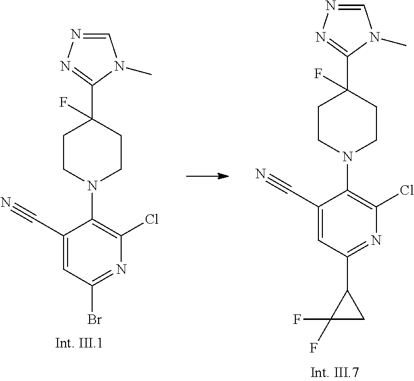 [SMILES: Cn1cnnc1C1(F)CCN(c2c(C#N)cc(Br)nc2Cl)CC1 ; Cn1cnnc1C1(F)CCN(c2c(C#N)cc(C3CC3(F)F)nc2Cl)CC1]

Under an argon atmosphere, Int. III.1 (150 mg, 0.36 mmol, 95% purity) and (2,2-difluorocyclopropyl) boronic acid (200 mg, 1.59 mmol, 97% purity) are suspended in 1.4-dioxane (3 mL). K₂CO₃ (aq. 2 M, 535 μL, 1.07 mmol) is added, and the resulting mixture is degassed for 10 min by passing an argon stream through the mixture. [1,1′-Bis-(diphenylphosphino)-ferrocen]-dichloro-palladium(II) (Pd(dppf)Cl₂) (26.1 mg, 0.04 mmol) is added, and the mixture is degassed again for 5 min. The mixture is then heated to 70° C. for 12 h. After cooling to ambient temperature, the mixture is purified by preparative HPLC (Sunfire C18, ACN/water gradient containing 0.1% TFA) to yield the title compound.

C₁₇H₁₆ClF₃N₆ (M=396.8 g/mol)

ESI-MS: 397 [M+H]⁺

Rt (HPLC): 0.56 min (method D)

### Intermediate III.8: 2-chloro-6-(1,1-difluoroethyl)-3-[4-fluoro-4-(4-methyl-4H-1,2,4-triazol-3-yl)piperidin-1-yl]pyridine-4-carbonitrile

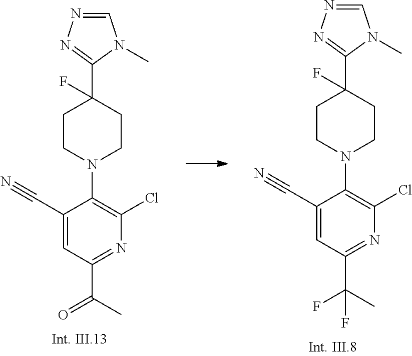 [SMILES: CC(=O)c1cc(C#N)c(N2CCC(F)(c3nncn3C)CC2)c(Cl)n1 ; Cn1cnnc1C1(F)CCN(c2c(C#N)cc(C(C)(F)F)nc2Cl)CC1]

Int. III.3 (300 mg, 0.83 mmol) is suspended in DCM (2 mL). DAST (400 μL, 3.03 mmol) is added and the resulting reaction mixture is stirred at rt for 2 days. The reaction is basified with an aqueous ammonia solution and the organic layer is separated, dried, and concentrated. The residue is purified by preparative HPLC (Xbridge C18, ACN/water gradient containing 0.1% NH₃) to yield the title compound.

C₁₆H₁₆ClF₃N₆ (M=384.8 g/mol)

ESI-MS: 385 [M+H]⁺

Rt (HPLC): 0.65 min (method B)

### Synthesis of Intermediate IV.1

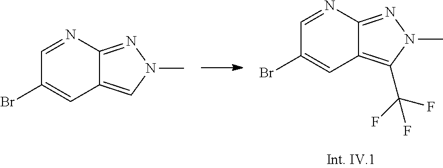

5-Bromo-2-methyl-2h-pyrazolo[3,4-b]pyridine (1.00 g, 4.72 mmol) and zinc(II) trifluoromethanesulfinate (2.35 g, 7.04 mmol) are suspended in a mixture of DCM (50 mL) and water (10 mL). TFA (351 μL, 4.72 mmol) and tert-butyl hydroperoxide (70% in water, 3.26 mL, 23.6 mmol) are added and the resulting reaction mixture is stirred at ambient temperature for 18 h. Zinc(II) trifluoromethanesulfinate (0.50 g, 1.51 mmol) and tert-butyl hydroperoxide (70% in water, 1.00 mL, 7.22 mmol) are added and the reaction mixture is stirred at 45° C. for 2 h. After cooling to ambient temperature, the reaction mixture is diluted with water and the organic phase is separated. The aqueous phase is extracted with DCM. The combined organic extracts are dried over MgSO₄, DMF (10 mL) is added and the mixture is concentrated. The residual DMF solution is purified by preparative HPLC (Sunfire C18, water/ACN gradient containing 0.1% NH₃) to yield the desired product along with other regioisomers from the trifluoromethylation reaction.

C₈H₅BrF₃N₃ (M=280.0 g/mol)

ESI-MS: 280/282 [M+H]⁺

Rt (HPLC): 1.01 min (method C)

### Synthesis of Intermediate IV.2

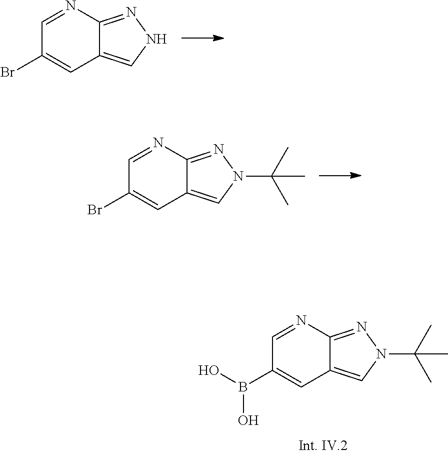 [SMILES: Brc1cnc2n[nH]cc2c1 ; CC(C)(C)n1cc2cc(Br)cnc2n1 ; CC(C)(C)n1cc2cc(B(O)O)cnc2n1]

### 5-Bromo-2-tert-butyl-2H-pyrazolo[3,4-b]pyridine

5-Bromo-2H-pyrazolo[3,4-b]pyridine (4.0 g, 19.8 mmol) is suspended in toluene (23 mL), and tert-butyl acetate (26.6 mL, 198 mmol) is added. Methanesulfonic acid (1.3 mL, 19.8 mmol) is added slowly. The resulting reaction mixture is heated to 80° C. and stirred for 1 h. After cooling to ambient temperature, additional methanesulfonic acid (1.3 mL, 19.8 mmol) is added, and the reaction mixture is heated to 80° C. and stirred for 1 h. After cooling to ambient temperature, the reaction mixture is concentrated and purified by preparative HPLC (XBridge C18, ACN/water gradient containing 0.1% TFA) to yield the 5-bromo-2-tert-butyl-2H-pyrazolo[3,4-b]pyridine.

C₁₀H₁₂BrN₃ (M=254.1 g/mol)

ESI-MS: 254/256 [M+H]⁺

Rt (HPLC): 0.50 min (method D)

### Intermediate IV.2: {2-tert-butyl-2H-pyrazolo[3,4-b]pyridin-5-yl} boronic acid

5-Bromo-2-tert-butyl-2H-pyrazolo[3,4-b]pyridine (1.50 g, 3.87 mmol), bis(pinacolato)diborane (1.21 g, 4.78 mmol), and potassium acetate (763 mg, 7.77 mmol) are added to 1,4-dioxane (15 mL), and the resulting mixture is degassed by passing an argon stream through the mixture for 10 min. [1,1′-Bis-(diphenylphosphino)-ferrocen]-dichloro-palladium (II) dichloromethane complex (Pd(dppf)Cl₂*CH₂Cl₂, CAS: 95464 May 4) (190 mg, 0.232 mmol) is added, and the mixture is degassed for additional 3 min. The mixture is then heated to 110° C. and stirred at this temperature for 4 h. After cooling to ambient temperature, the mixture is concentrated, and the residue is suspended in an ACN/water mixture, filtered, and purified by preparative HPLC (XBridge C18, ACN/water gradient containing 0.1% TFA) to yield the title compound.

C₁₀H₁₄BN₃O₂ (M=219.1 g/mol)

ESI-MS: 220 [M+H]⁺

Rt (HPLC): 0.27 min (method D)

### Synthesis of Intermediate IV.3

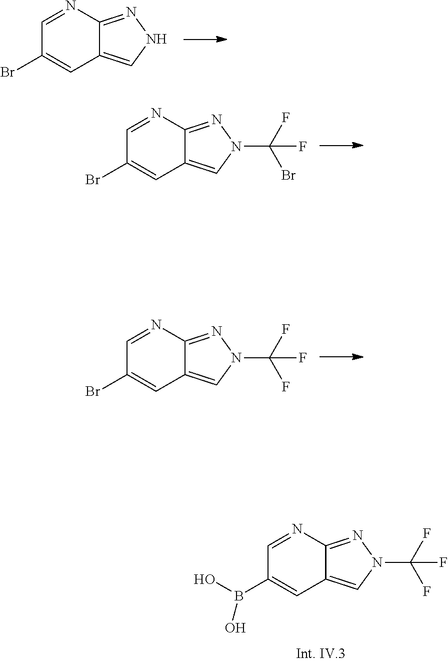 [SMILES: Brc1cnc2n[nH]cc2c1 ; FC(F)(Br)n1cc2cc(Br)cnc2n1 ; FC(F)(F)n1cc2cc(Br)cnc2n1 ; OB(O)c1cnc2nn(C(F)(F)F)cc2c1]

### 5-Bromo-2-(bromodifluoromethyl)-2H-pyrazolo[3,4-b]pyridine

5-Bromo-2H-pyrazolo[3,4-b]pyridine (6.00 g, 28.8 mmol) is dissolved in DMF (200 mL). At 0° C., NaH (55%, 1.51 g, 34.5 mmol) is added, and the reaction mixture is stirred at 0° C. for 30 min. Then dibromodifluoromethane (8.30 mL, 86.4 mmol) is added, and the reaction mixture is stirred at rt overnight. The reaction mixture is diluted with ACN/H₂O and purified by preparative HPLC (XBridge C18, ACN/water gradient containing 0.1% TFA) to yield the desired compound.

C₇H₃Br₂F₂N₃ (M=326.9 g/mol)

ESI-MS: 326/328/330 [M+H]⁺

Rt (HPLC): 0.56 min (method D)

### 5-Bromo-2-(trifluoromethyl)-2H-pyrazolo[3,4-b]pyridine

5-Bromo-2-(bromodifluoromethyl)-2H-pyrazolo[3,4-b]pyridine (2.10 g, 6.42 mmol) and silver tetrafluoroborate (2.53 g, 12.8 mmol) are dissolved in DCM (40 mL) and stirred at 80° C. overnight. After cooling to ambient temperature, the reaction mixture is concentrated and purified by column chromatography (SiO₂, DCM/MeOH gradient 1:0 to 1:1) to afford the desired compound.

C₇H₃BrF₃N₃ (M=266.0 g/mol)

ESI-MS: 266/268 [M+H]⁺

Rt (HPLC): 0.47 min (method D)

### Intermediate IV.3: [2-(trifluoromethyl)-2H-pyrazolo[3,4-b]pyridin-5-yl]boronic acid

5-Bromo-2-(trifluoromethyl)-2H-pyrazolo[3,4-b]pyridine (324 mg, 0.61 mmol, 50% purity), bis(pinacolato)diborane (231 mg, 0.91 mmol), and potassium acetate (179 mg, 1.83 mmol) are added to 1,4-dioxane (3 mL), and the resulting mixture is degassed by passing an argon stream through the mixture for 10 min. [1,1′-Bis-(diphenylphosphino)-ferrocen]-dichloro-palladium (II) dichloromethane complex (Pd(dppf)Cl₂*CH₂Cl₂, CAS: 95464 May 4) (49.7 mg, 0.06 mmol) is added, and the mixture is then heated to 90° C. and stirred at this temperature for 5 h. After cooling to ambient temperature, the mixture is concentrated, and the residue is dissolved in an ACN/water mixture, filtered, and purified by preparative HPLC (XBridge C18, ACN/water gradient containing 0.1% TFA) to yield the title compound.

C₇H₅BF₃N₃O₂ (M=230.9 g/mol)

ESI-MS: 232 [M+H]⁺

Rt (HPLC): 0.30 min (method D)

### Synthesis of Intermediate V.1: 2-chloro-3-[4-fluoro-4-(4-methyl-4H-1,2,4-triazol-3-yl)piperidin-1-yl]-6-(1-methyl-1H-pyrazol-4-yl)pyridine-4-carbonitrile

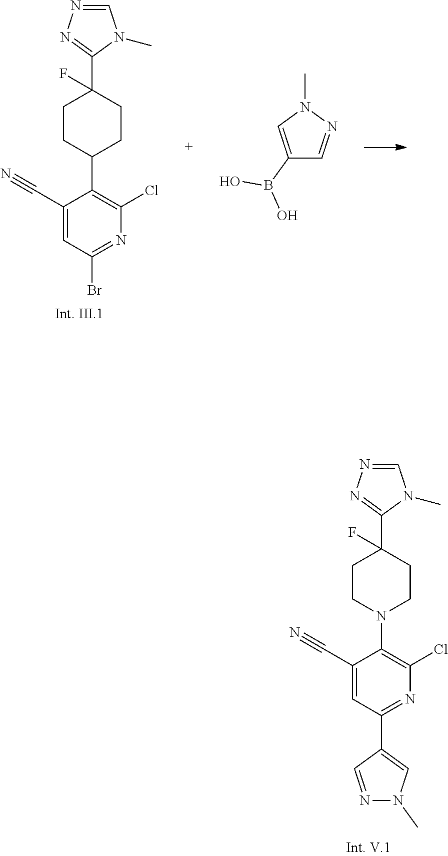

To a mixture of Int. III.1 (30.0 mg, 58.4 μmol), 1-methyl-1H-pyrazole-4-boronic acid (8.1 mg, 64.2 mmol) and potassium carbonate (32.3 mg, 234 μmol) water (100 μL) and toluene. The resulting mixture is purged by passing an argon stream through the mixture for 10 min. Pd(dtbpf)Cl2 (1.0 mg, 1.5 μmol) is added, and the mixture is further purged for 5 min. The reaction mixture is stirred at 75° C. overnight. After cooling to ambient temperature, the mixture is diluted with dichloromethane and extracted with water. The organic extract is concentrated and purified by preparative HPLC (XBridge C18 column, ACN/water gradient containing 0.1% NH₃) to yield the title compound.

C₁₈H₁₈ClFN₈ (M=400.8 g/mol)

ESI-MS: 401 [M+H]⁺

Rt (HPLC): 0.52 min (method B)

### Intermediates synthesized analogous to the procedure described for Int. V.1

| | | | | Molecular |
|---|---|---|---|---|
| | | | | Formula (MW) |
| | | | | ESI-MS |
| | | | Deviation from | HPLC |
| | | | general | retention time |
| Int. | Starting material | Structure | procedure | (method) |
| V.2 | Int. III.1 + 1,3-dimethyl-4-(4,4,5,5- tetramethyl-1,3,2- dioxaborolan-2-yl)-1H- pyrazole | 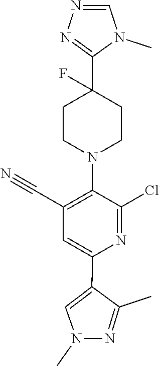 [SMILES: Cc1nn(C)cc1-c1cc(C#N)c(N2CCC(F)(c3nncn3C)CC2)c(Cl)n1] | None. | C₁₉H₂₀ClFN₈ (M = 414.9 g/mol) ESI-MS: 415 [M + H]⁺ Rt (HPLC): 0.56 min (method B) |
| V.3 | Int. III.1 + (1,3,5-trimethyl-1H- pyrazol-4-yl) boronic acid | 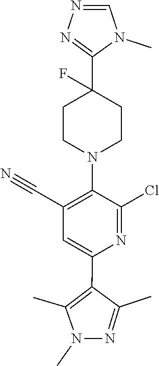 [SMILES: Cc1nn(C)c(C)c1-c1cc(C#N)c(N2CCC(F)(c3nncn3C)CC2)c(Cl)n1] | Pd(dppf)Cl2 was used as catalyst; reaction temperature 65° C.; purification by prep. HPLC (Sunfire C18, MeCN/water gradient containing 0.1% TFA) | C₂₀H₂₂ClFN₈ (M = 428.9 g/mol) ESI-MS: 429 [M + H]⁺ Rt (HPLC): 0.73 min (method E) |
| V.4 | Int. III.1 + 4-fluorophenyl boronic acid | 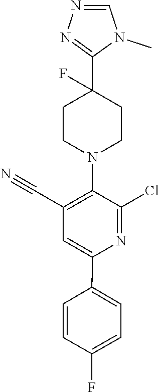 [SMILES: Cn1cnnc1C1(F)CCN(c2c(C#N)cc(-c3ccc(F)cc3)nc2Cl)CC1] | Pd(dppf)Cl2 was used as catalyst; Dioxane was used as solvent. Reaction temperature: 70° C. purified by column chromatography (SiO₂, EtOAc/MeOH gradient) | C₂₀H₁₇ClF₂N₆ (M = 414.8 g/mol) ESI-MS: 415 [M + H]⁺ Rt (HPLC): 0.98 min (method C) |
| V.5 | Int. III.1 + 4-(4,4,5,5,-tetramethyl- 1,3,2-dioxaborolan-2- yl)pyridazine | 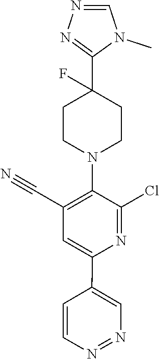 [SMILES: Cn1cnnc1C1(F)CCN(c2c(C#N)cc(-c3ccnnc3)nc2Cl)CC1] | 80° C. overnight; 2^{nd} and 3^{rd} crops of catalyst and boronic acid ester added before additional 80° C. overnight reactions. Purified by prep. HPLC (Sunfire C18, acetonitrile gradient containing 0.1% TFA) | C₁₈H₁₆ClFN₈ (M = 398.8 g/mol) ESI-MS: 399 [M + H]⁺ Rt (HPLC): 0.55 min (method E) |
| V.6 | Int. III.1 + (2-methylpyrimidin-5- yl)boronic acid | 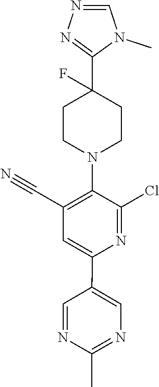 [SMILES: Cc1ncc(-c2cc(C#N)c(N3CCC(F)(c4nncn4C)CC3)c(Cl)n2)cn1] | 80° C. overnight; 2^{nd} crops of catalyst and boronic acid added before second 80° C. overnight reaction. Purified by prep. HPLC (Sunfire C18, acetonitrile gradient containing 0.1% TFA) | C₁₉H₁₈ClFN₈ (M = 412.9 g/mol) ESI-MS: 413 [M + H]⁺ Rt (HPLC): 0.67 min (method E) |
| V.7 | Int. III.1 + (2- (trifluoromethyl)pyrimidin- 5-yl)boronic acid | 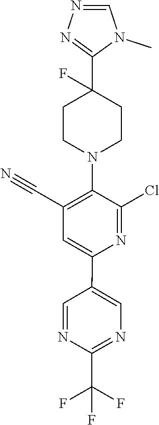 [SMILES: Cn1cnnc1C1(F)CCN(c2c(C#N)cc(-c3cnc(C(F)(F)F)nc3)nc2Cl)CC1] | Reaction temperature: 80° C.; Purification by preparative HPLC (Sunfire C18, MeCN/water gradient containing 0.1% TFA) | C₁₉H₁₅ClF₄N₈ (M = 466.8 g/mol) ESI-MS: 467 [M + H]⁺ Rt (HPLC): 0.83 min (method E) |
| V.8 | Int. III.1 + 6-methylpyridine-3- boronic acid | 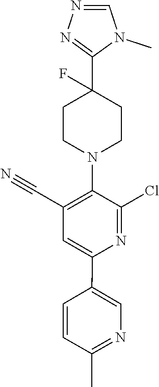 [SMILES: Cc1ccc(-c2cc(C#N)c(N3CCC(F)(c4nncn4C)CC3)c(Cl)n2)cn1] | 80° C. overnight; 2^{nd} crops of catalyst and boronic acid added before second 80° C. overnight reaction. | C₂₀H₁₉ClFN₇ (M = 411.9 g/mol) ESI-MS: 412 [M + H]⁺ Rt (HPLC): 0.49 min (method E) |
| V.9 | Int. III.1 + 3-(4,4,5,5-tetramethyl- 1,3,2-dioxaborolan-2- yl)pyridine | 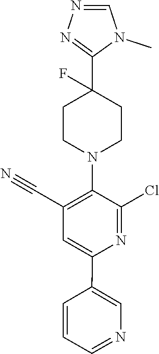 [SMILES: Cn1cnnc1C1(F)CCN(c2c(C#N)cc(-c3cccnc3)nc2Cl)CC1] | Reaction temperature: 80° C. Purification by preparative HPLC (Sunfire C18, MeCN/water gradient containing 0.1% TFA) | C₁₉H₁₇ClFN₇ (M = 397.8 g/mol) ESI-MS: 398 [M + H]⁺ Rt (HPLC): 0.46 min (method E) |
| V.10 | Int. III.1 + 2-Methyl-5-(4,4,5,5- tetramethyl-1,3,2- dioxaborolan-2-yl)oxazole | 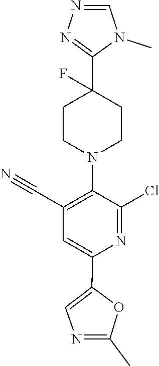 [SMILES: Cc1ncc(-c2cc(C#N)c(N3CCC(F)(c4nncn4C)CC3)c(Cl)n2)o1] | Reaction temperature: 80° C. Purification by preparative HPLC (Sunfire C18, MeCN/water gradient containing 0.1% TFA) | C₁₈H₁₇ClFN₇O (M = 401.8 g/mol) ESI-MS: 402 [M + H]⁺ Rt (HPLC): 0.66 min (method E) |

### Preparation of Final Compounds

### Example 1

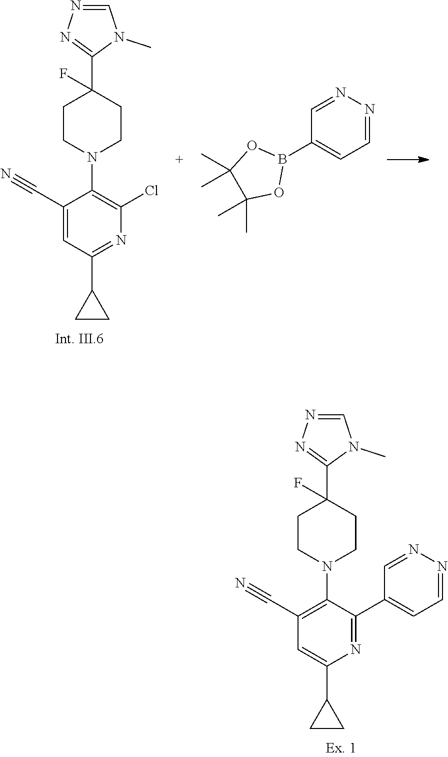

To a mixture of Int. III.6 (50.0 mg, 139 μmol) and 4-(4,4,5,5-tetramethyl-1,3,2-dioxaborolan-2-yl)pyridazine (44.2 mg, 208 μmol) in 1,4-dioxane (2 mL) is added an aqueous potassium carbonate solution (2 M, 208 μL, 0.416 mmol). The resulting mixture is degassed by passing an argon stream through the mixture for 2 min. Pd(dppf)Cl₂ (10.1 mg, 13.9 μmol) is added, and the mixture is further purged for 2 min. The reaction mixture is stirred at 100° C. for 5 h. After cooling to ambient temperature, the mixture is concentrated and purified by column chromatography (SiO₂, EtOAc/MeOH gradient). After concentration of product containing fractions, the residue is washed with Et₂O to yield the desired product.

### Examples Synthesized Analogous to the Procedure Described for Example 1

| ex- | Starting | | Deviation from general |
|---|---|---|---|
| ample | materials | Structure | procedure |
| 2 | III.6 + Int. IV.3 | 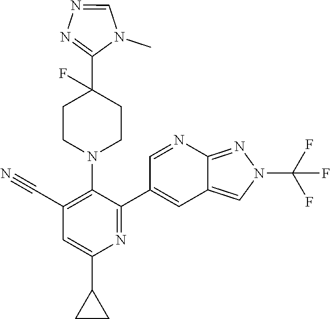 [SMILES: Cn1cnnc1C1(F)CCN(c2c(C#N)cc(C3CC3)nc2-c2cnc3nn(C(F)(F)F)cc3c2)CC1] | — |
| 3 | III.8 + Int. IV.3 | 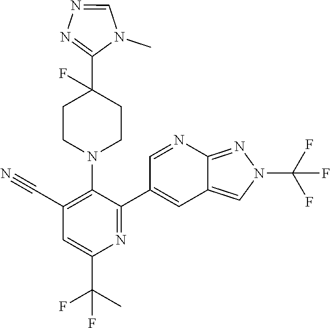 [SMILES: Cn1cnnc1C1(F)CCN(c2c(C#N)cc(C(C)(F)F)nc2-c2cnc3nn(C(F)(F)F)cc3c2)CC1] | 3 h at 95° C.; Purification by preparative HPLC (XBridge C18, MeCN/water gradient containing 0.1% NH3) |
| 5 | III.2 + Int. IV.3 | 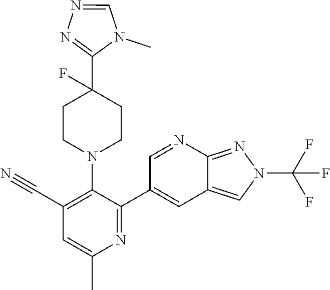 [SMILES: Cc1cc(C#N)c(N2CCC(F)(c3nncn3C)CC2)c(-c2cnc3nn(C(F)(F)F)cc3c2)n1] | — |
| 6 | III.7 + 4-(4,4,5,5- tetramethyl- 1,3,2- dioxaborolan- 2-yl)pyridazine | 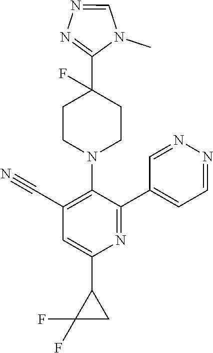 [SMILES: Cn1cnnc1C1(F)CCN(c2c(C#N)cc(C3CC3(F)F)nc2-c2ccnnc2)CC1] | — |
| 12 | III.6 + 3-methyl-5- (4,4,5,5- tetramethyl- 1,3,2- dioxaborolan- 2-yl)pyridazine | 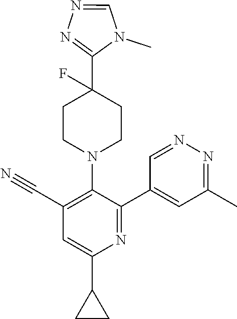 [SMILES: Cc1cc(-c2nc(C3CC3)cc(C#N)c2N2CCC(F)(c3nncn3C)CC2)cnn1] | 18 h at 85° C. |

### Synthesis of Example 9

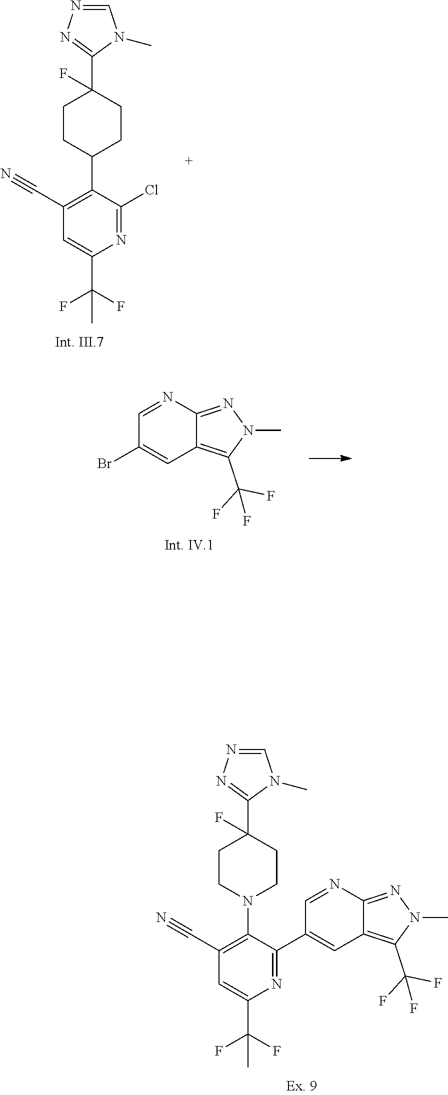 [SMILES: Cn1cnnc1C1(F)CCC(c2c(C#N)cc(C(C)(F)F)nc2Cl)CC1 ; Cn1nc2ncc(Br)cc2c1C(F)(F)F ; Cn1cnnc1C1(F)CCN(c2c(C#N)cc(C(C)(F)F)nc2-c2cnc3nn(C)c(C(F)(F)F)c3c2)CC1]

A mixture of Intermediate IV.1 (60.0 mg, 183 μmol), bis(pinacolato)diboron (63.3 mg, 249 μmol) and potassium acetate (49.0 mg, 499 μmol) in dioxane (2 mL) is degassed by passing an Ar stream for 5 min. Pd(dppf)Cl_{2*}CH₂Cl₂ (2.7 mg, 3.3 μmol) is added and the reaction mixture is stirred for 4 h at 100° C. After cooling to ambient temperature, an aqueous solution of sodium carbonate (2 M, 231.5 μL, 463 μmol) and Int. III.7 (60.0 mg, 166 μmol) is added. The mixture is degassed by passing an Ar stream for 5 min. Pd(dppf)Cl_{2*}CH₂Cl₂ (2.7 mg, 3.3 μmol) is added and the reaction mixture is stirred for 4 h at 100° C. After cooling to ambient temperature, the reaction mixture concentrated and purified by column chromatography (SiO₂, EtOAc/MeOH gradient) to yield the desired product.

### Examples Synthesized Analogous to the Procedure Described for Example 9

| ex- | Starting | | Deviation from general |
|---|---|---|---|
| ample | materials | Structure | procedure |
| 10 | III.4 + Int. IV.1 | 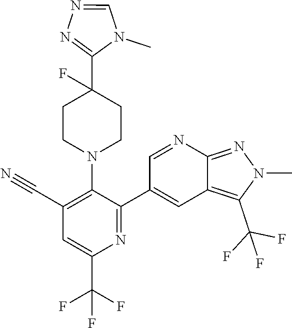 [SMILES: Cn1cnnc1C1(F)CCN(c2c(C#N)cc(C(F)(F)F)nc2-c2cnc3nn(C)c(C(F)(F)F)c3c2)CC1] | — |
| 11 | III.6 + Int. IV.1 | 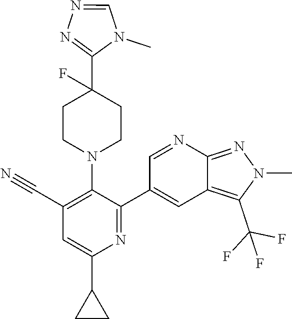 [SMILES: Cn1cnnc1C1(F)CCN(c2c(C#N)cc(C3CC3)nc2-c2cnc3nn(C)c(C(F)(F)F)c3c2)CC1] | — |

### Synthesis of Examples 14, 15 and 16

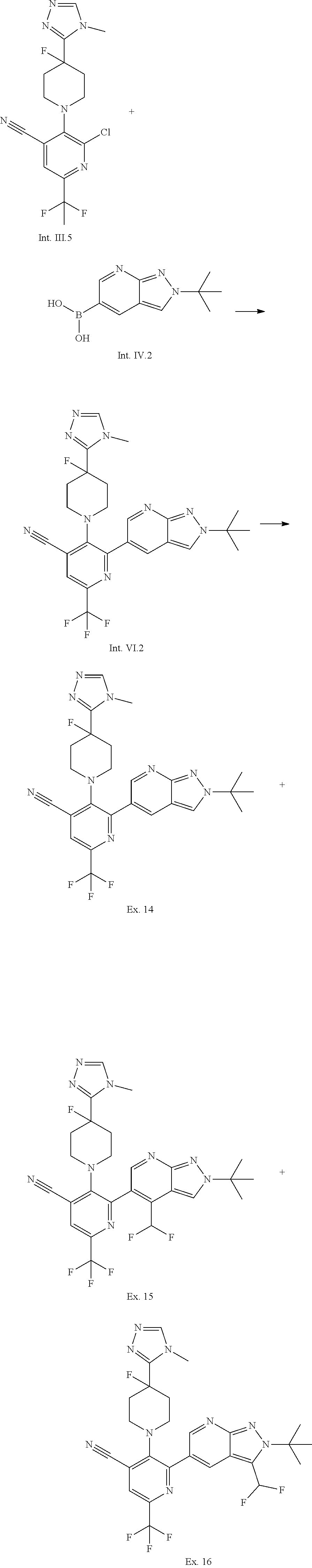 [SMILES: Cn1cnnc1C1(F)CCN(c2c(C#N)cc(C(C)(F)F)nc2Cl)CC1 ; CC(C)(C)n1cc2cc(B(O)O)cnc2n1 ; Cn1cnnc1C1(F)CCN(c2c(C#N)cc(C(F)(F)F)nc2-c2cnc3nn(C(C)(C)C)cc3c2)CC1 ; Cn1cnnc1C1(F)CCN(c2c(C#N)cc(C(F)(F)F)nc2-c2cnc3nn(C(C)(C)C)cc3c2)CC1 ; Cn1cnnc1C1(F)CCN(c2c(C#N)cc(C(F)(F)F)nc2-c2cnc3nn(C(C)(C)C)cc3c2C(F)F)CC1 ; Cn1cnnc1C1(F)CCN(c2c(C#N)cc(C(F)(F)F)nc2-c2cnc3nn(C(C)(C)C)c(C(F)F)c3c2)CC1]

### Intermediate VI.2

To a solution of Int. III.5 (50.0 mg, 0.12 mmol) and Int. IV.2 (46.1 mg, 0.14 mmol) in 1,4-dioxane (2 mL) is added potassium carbonate (2 M in water, 0.17 mL, 0.35 mmol). The resulting mixture is purged by passing an argon stream through the solution. [1,1′-Bis-(diphenylphosphino)-ferrocen]-dichloro-palladium (II) (Pd(dppf)Cl₂, CAS: 72287-26-4) (8.5 mg, 11.5 μmol) is added, and the mixture is further purged with argon. The reaction mixture is stirred at 95° C. for 4.5 h. After cooling to ambient temperature, the mixture is diluted with an ACN/water mixture and purified by preparative HPLC (XBridge C18 column, ACN/water gradient containing 0.1% NH₃) to yield the title compound.

C₂₅H₂₅F₄N₉ (M=527.5 g/mol)

ESI-MS: 528 [M+H]⁺

Rt (HPLC): 0.65 min (method B)

### Examples 14, 15 and 16

To a mixture Int. VI.2 (400 mg, 758 μmol) in dichloromethane (8 mL) and water (4 mL) is added zinc(II) difluoromethanesulfinate (672 mg, 2.27 mmol), trifluoroacetic acid (58.5 μL, 758 μmol) and tert-butyl hydroperoxide (70%, 519 μL, 3.79 mmol). The resulting reaction mixture is stirred at ambient temperature for 18 h. The mixture is concentrated and the residue purified by preparative HPLC (X-Bridge C18 column, MeCN/water gradient containing 0.1% NH₃) and further purified by column chromatography (SiO₂, EtOAc/MeOH gradient) and preparative SFC (Torus_1AA, MeOH/CO2, 40° C., 120 bar) to yield the title compounds.

### Example 19

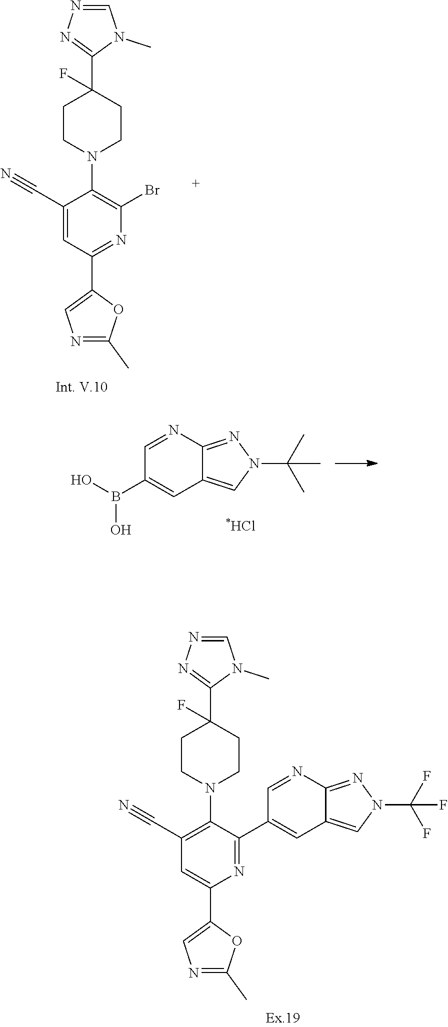 [SMILES: Cc1ncc(-c2cc(C#N)c(N3CCC(F)(c4nncn4C)CC3)c(Br)n2)o1 ; CC(C)(C)n1cc2cc(B(O)O)cnc2n1 ; Cc1ncc(-c2cc(C#N)c(N3CCC(F)(c4nncn4C)CC3)c(-c3cnc4nn(C(F)(F)F)cc4c3)n2)o1]

To a mixture of Int. V.10 (40.0 mg, 77.5 μmol) and Int. IV.3 (25.1 mg, 93.1 μmol) in 1,4-dioxane (1.5 mL) is added an aqueous potassium carbonate solution (2 M, 155 μL, 0.311 mmol). The resulting mixture is purged by passing an argon stream through the mixture. Pd(dppf)Cl₂ (5.7 mg, 7.8 μmol) is added, and the mixture is further purged with argon for 5 min. The reaction mixture is stirred at 95° C. for 18 h. After cooling to ambient temperature, the mixture is diluted with an ACN mixture, filtered, and purified by preparative HPLC (Sunfire C18 column, ACN/water gradient containing 0.1% TFA) to yield the title compound.

### Examples Synthesized Analogous to the Procedure Described for Example 19

| ex- | Starting | | Deviation from general |
|---|---|---|---|
| ample | materials | Structure | procedure |
| 17 | V.2 + IV.3 | 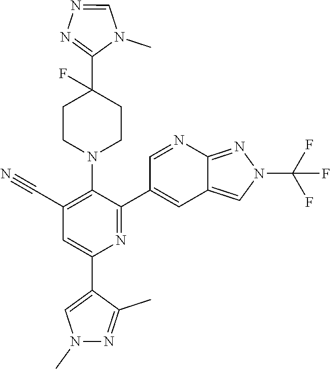 [SMILES: Cc1nn(C)cc1-c1cc(C#N)c(N2CCC(F)(c3nncn3C)CC2)c(-c2cnc3nn(C(F)(F)F)cc3c2)n1] | — |
| 18 | V.4 + 4-(4,4,5,5- tetramethyl- 1,3,2- dioxaborolan-2- yl)pyridazine | 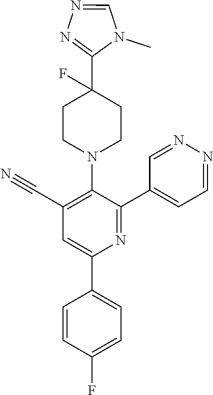 [SMILES: Cn1cnnc1C1(F)CCN(c2c(C#N)cc(-c3ccc(F)cc3)nc2-c2ccnnc2)CC1] | 2 eq. boronic acid ester, 2.0 eq. of K₂CO₃ solution; reaction temperature 85° C.; Purified by column chromatography (SiO₂, EtOAc/MeOH gradient) |
| 20 | V.7 + IV.3 | 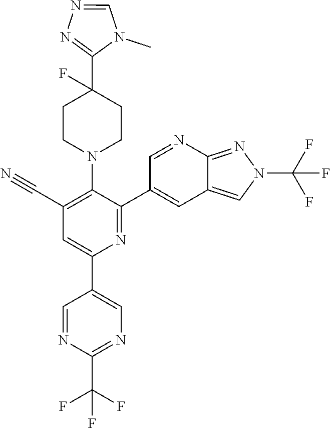 [SMILES: Cn1cnnc1C1(F)CCN(c2c(C#N)cc(-c3cnc(C(F)(F)F)nc3)nc2-c2cnc3nn(C(F)(F)F)cc3c2)CC1] | — |
| 21 | V.9 + IV.3 | 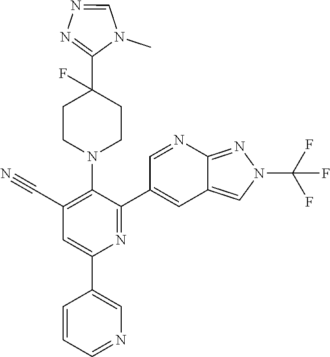 [SMILES: Cn1cnnc1C1(F)CCN(c2c(C#N)cc(-c3cccnc3)nc2-c2cnc3nn(C(F)(F)F)cc3c2)CC1] | Purified by preparative HPLC (XBridge C18 column, MeCN/water containing 0.1% NH₃) |
| 22 | V.8 + IV.3 | 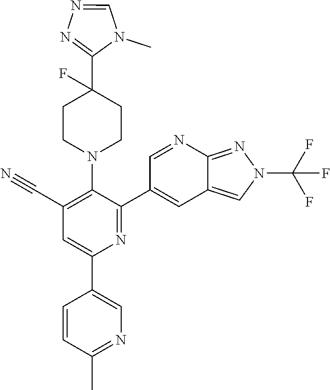 [SMILES: Cc1ccc(-c2cc(C#N)c(N3CCC(F)(c4nncn4C)CC3)c(-c3cnc4nn(C(F)(F)F)cc4c3)n2)cn1] | Purified by preparative HPLC (XBridge C18 column, MeCN/water containing 0.1% NH₃) |
| 23 | V.3 + IV.3 | 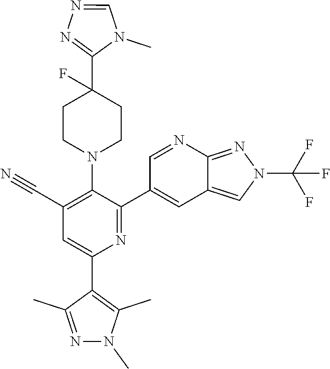 [SMILES: Cc1nn(C)c(C)c1-c1cc(C#N)c(N2CCC(F)(c3nncn3C)CC2)c(-c2cnc3nn(C(F)(F)F)cc3c2)n1] | Reaction time: 3 h at 95° C. |
| 24 | V.1 + IV.3 | 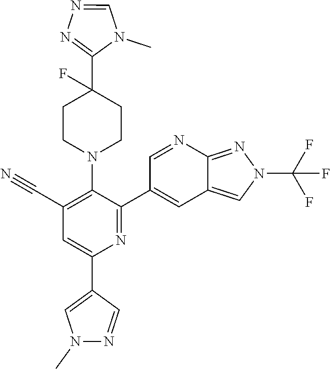 [SMILES: Cn1cc(-c2cc(C#N)c(N3CCC(F)(c4nncn4C)CC3)c(-c3cnc4nn(C(F)(F)F)cc4c3)n2)cn1] | — |
| 25 | V.6 + IV.3 | 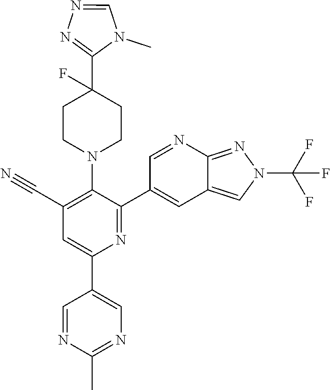 [SMILES: Cc1ncc(-c2cc(C#N)c(N3CCC(F)(c4nncn4C)CC3)c(-c3cnc4nn(C(F)(F)F)cc4c3)n2)cn1] | — |

### Synthesis of Example 26

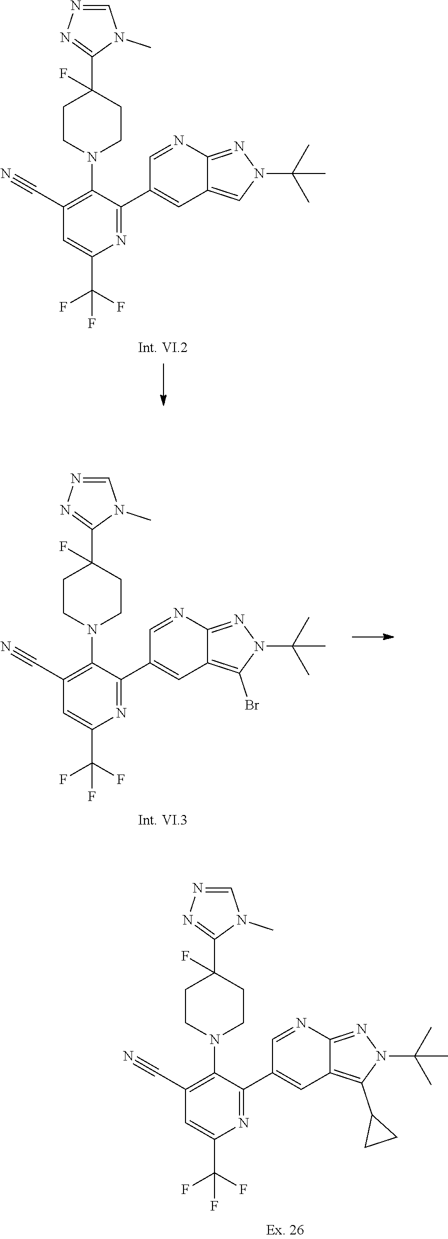 [SMILES: Cn1cnnc1C1(F)CCN(c2c(C#N)cc(C(F)(F)F)nc2-c2cnc3nn(C(C)(C)C)cc3c2)CC1 ; Cn1cnnc1C1(F)CCN(c2c(C#N)cc(C(F)(F)F)nc2-c2cnc3nn(C(C)(C)C)c(Br)c3c2)CC1 ; Cn1cnnc1C1(F)CCN(c2c(C#N)cc(C(F)(F)F)nc2-c2cnc3nn(C(C)(C)C)c(C4CC4)c3c2)CC1]

### Int. VI.3

To a mixture of Int. VI.2 (50 mg, 94.8 μmol) in acetonitrile (1.0 mL) is added N-bromo succinimide (25.3 mg, 142 μmol). The resulting reaction mixture is stirred for 12 h at 70° C. After cooling to ambient temperature, the reaction mixture is concentrated and the residue is purified by column chromatography (SiO₂, EtOAC/MeOH gradient) to yield the title compound.

C₂₅H₂₄BrF₄N₉ (M=606.4 g/mol)

ESI-MS: 606/608 [M+H]⁺

Rt (HPLC): 1.00 min (method C)

### Example 26

To a mixture of Int. VI.3 (50.0 mg, 82.5 μmol) and cyclopropylboronic acid (28.3 mg, 330 μmol) in 1,4-dioxane (2.0 mL) is added an aqueous potassium carbonate solution (2 M, 82 μL, 0.165 mmol). The resulting mixture is purged by passing an argon stream through the mixture. Pd(dppf)Cl₂ (5.7 mg, 7.8 μmol) is added, and the mixture is further purged with argon for 5 min. The reaction mixture is stirred at 85° C. for 18 h. After cooling to ambient temperature, the mixture is concentrated and purified by column chromatography (SiO₂. EtOAc/MeOH gradient) to yield the title compound.

### Analytical Data of Synthesized Examples

| | | Molecular Formula | |
|---|---|---|---|
| | | (MW) | |
| | | ESI-MS | |
| ex- | | HPLC retention time | ¹H NMR (400 MHz, |
| ample | Structure | (method) | DMSO-d6): δ in ppm |
| 1 | 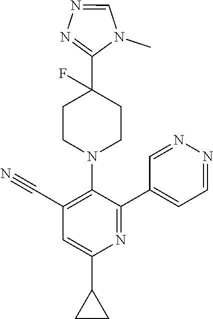 [SMILES: Cn1cnnc1C1(F)CCN(c2c(C#N)cc(C3CC3)nc2-c2ccnnc2)CC1] | C₂₁H₂₁FN₈ (M = 404.5 g/mol) ESI-MS: 405 [M + H]⁺ Rt (HPLC): 0.76 min (method C) | 9.47 (dd, J = 2.2, 1.3 Hz, 1 H), 9.37 (dd, J = 5.3, 1.2 Hz, 1 H), 8.48 (s, 1 H), 7.90 (dd, J = 5.3, 2.3 Hz, 1 H), 7.85 (s, 1 H), 3.73 (d, J = 1.5 Hz, 3 H), 3.24- 3.34 (m, 2 H), 3.07- 3.16 (m, 2 H), 2.04- 2.27 (m, 5 H), 0.93- 1.06 (m, 4 H) |
| 2 | 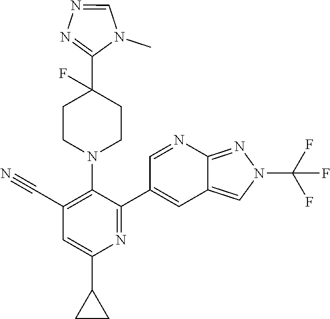 [SMILES: Cn1cnnc1C1(F)CCN(c2c(C#N)cc(C3CC3)nc2-c2cnc3nn(C(F)(F)F)cc3c2)CC1] | C₂₄H₂₁F₄N₉ (M = 511.5 g/mol) ESI-MS: 512 [M + H]⁺ Rt (HPLC): 0.93 min (method C) | 9.30 (s, 1 H), 9.07 (d, J = 2.2 Hz, 1 H), 8.42- 8.51 (m, 2 H), 7.78 (s, 1 H), 3.70 (d, J = 1.4 Hz, 3 H), 3.25-3.35 (m, 2 H), 3.11-3.22 (m, 2 H), 2.03-2.29 (m, 5 H), 0.93-1.06 (m, 4 H) |
| 3 | 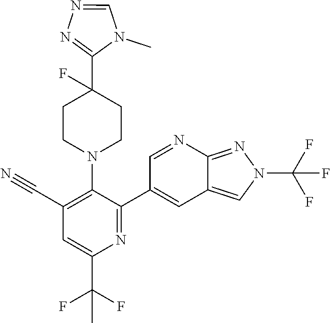 [SMILES: Cn1cnnc1C1(F)CCN(c2c(C#N)cc(C(C)(F)F)nc2-c2cnc3nn(C(F)(F)F)cc3c2)CC1] | C₂₃H₁₉F₆N₉ (M = 535.5 g/mol) ESI-MS: 536 [M + H]⁺ Rt (HPLC): 0.66 min (method B) | 9.35 (s, 1 H), 9.12 (d, J = 2.3 Hz, 1 H), 8.56 (d, J = 2.3 Hz, 1 H), 8.47 (s, 1 H), 8.13 (s, 1 H), 3.71 (d, J = 1.5 Hz, 3 H), 3.30-3.40 (m, 4 H), 2.11-2.31 (m, 4 H), 2.03 (t, J = 19.0 Hz, 3 H) |
| 5 | 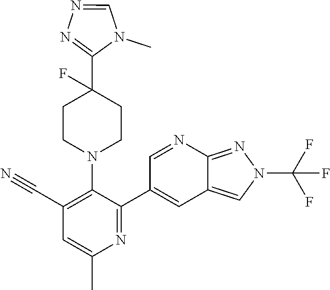 [SMILES: Cc1cc(C#N)c(N2CCC(F)(c3nncn3C)CC2)c(-c2cnc3nn(C(F)(F)F)cc3c2)n1] | C₂₂H₁₉F₄N₉ (M = 485.4 g/mol) ESI-MS: 486 [M + H]⁺ Rt (HPLC): 0.83 min (method C) | 9.31 (s, 1 H), 9.08 (d, J = 2.3 Hz, 1 H), 8.48 (d, J = 2.3 Hz, 1 H), 8.46 (s, 1 H), 7.75 (s, 1 H), 3.70 (d, J = 1.5 Hz, 3 H), 3.26-3.35 (m, 2 H), 3.14-3.25 (m, 2 H), 2.54 (s, 3 H), 2.03- 2.26 (m, 4 H) |
| 6 | 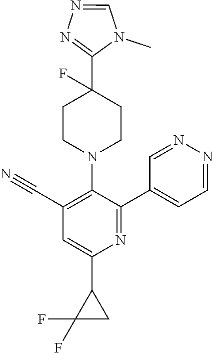 [SMILES: Cn1cnnc1C1(F)CCN(c2c(C#N)cc(C3CC3(F)F)nc2-c2ccnnc2)CC1] | C₂₁H₁₉F₃N₈ (M = 440.4 g/mol) ESI-MS: 441 [M + H]⁺ Rt (HPLC): 0.79 min (method C) | 9.50 (dd, J = 2.3, 1.3 Hz, 1 H), 9.41 (dd, J = 5.3, 1.2 Hz, 1 H), 8.49 (s, 1 H), 8.00 (s, 1 H), 7.92 (dd, J = 5.3, 2.3 Hz, 1 H), 3.74 (d, J = 1.5 Hz, 3 H), 3.35-3.27 (m, 1 H), 3.16-3.29 (m, 4 H), 2.28-2.39 (m, 1 H), 2.03-2.27 (m, 5 H) |
| 9 | 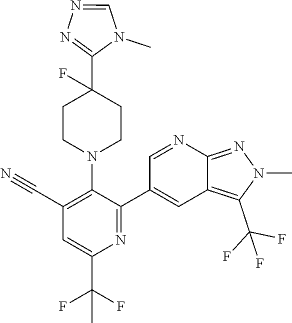 [SMILES: Cn1cnnc1C1(F)CCN(c2c(C#N)cc(C(C)(F)F)nc2-c2cnc3nn(C)c(C(F)(F)F)c3c2)CC1] | C₂₄H₂₁F₆N₉ (M = 549.5 g/mol) ESI-MS: 550 [M + H]⁺ Rt (HPLC): 0.94 min (method C) | 9.03 (d, J = 2.2 Hz, 1 H), 8.49 (dd, J = 1.9, 1.1 Hz, 1 H), 8.46 (s, 1 H), 8.12 (s, 1 H), 4.39 (d, J = 0.8 Hz, 3 H), 3.70 (d, J = 1.5 Hz, 3 H), 3.22-3.35 (m, 4 H), 2.10-2.24 (m, 4 H), 2.02 (t, J = 19.0 Hz, 3 H) |
| 10 | 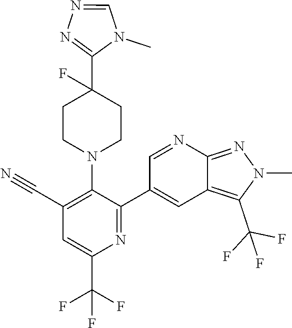 [SMILES: Cn1cnnc1C1(F)CCN(c2c(C#N)cc(C(F)(F)F)nc2-c2cnc3nn(C)c(C(F)(F)F)c3c2)CC1] | C₂₃H₁₈F₇N₉ (M = 553.4 g/mol) ESI-MS: 554 [M + H]⁺ Rt (HPLC): 0.95 min (method C) | 9.00 (d, J = 2.0 Hz, 1 H), 8.50 (dd, J = 1.9, 1.1 Hz, 1 H), 8.46 (s, 1 H), 8.41 (s, 1 H), 4.40 (d, J = 0.8 Hz, 3 H), 3.70 (d, J = 1.5 Hz, 3 H), 3.35-3.42 (m, 2 H), 3.23-3.29 (m, 2 H), 2.07-2.25 (m, 4 H) |
| 11 | 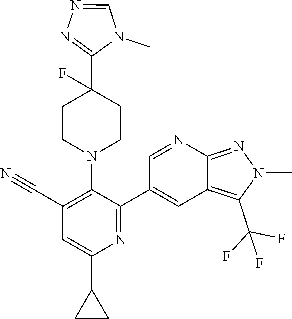 [SMILES: Cn1cnnc1C1(F)CCN(c2c(C#N)cc(C3CC3)nc2-c2cnc3nn(C)c(C(F)(F)F)c3c2)CC1] | C₂₅H₂₃F₄N₉ (M = 525.5 g/mol) ESI-MS: 526 [M + H]⁺ Rt (HPLC): 0.96 min (method C) | 8.98 (d, J = 2.2 Hz, 1 H), 8.45 (s, 1 H), 8.40-8.43 (m, 1 H), 7.76 (s, 1 H), 4.38 (d, J = 0.6 Hz, 3 H), 3.70 (d, J = 1.4 Hz, 3 H), 3.25 (br d, J = 9.6 Hz, 2 H), 3.11-3.20 (m, 2 H), 2.19-2.26 (m, 1 H), 2.00-2.19 (m, 4 H), 0.93-1.05 (m, 4 H) |
| 12 | 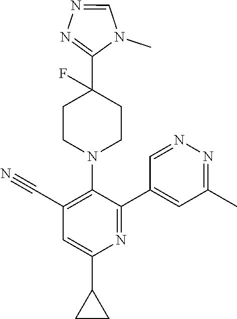 [SMILES: Cc1cc(-c2nc(C3CC3)cc(C#N)c2N2CCC(F)(c3nncn3C)CC2)cnn1] | C₂₂H₂₃FN₈ (M = 418.5 g/mol) ESI-MS: 419 [M + H]⁺ Rt (HPLC): 0.79 min (method C) | 9.28 (d, J = 1.9 Hz, 1 H), 8.48 (s, 1 H), 7.83 (s, 1 H), 7.76 (d, J = 2.0 Hz, 1 H), 3.73 (d, J = 1.6 Hz, 3 H), 3.23- 3.29 (m, 2 H), 3.06- 3.15 (m, 2 H), 2.69 (s, 3 H), 2.05-2.26 (m, 5 H), 0.92-1.06 (m, 4 H) |
| 14 | 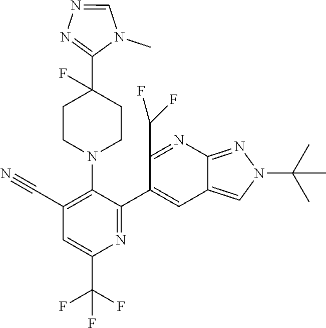 [SMILES: Cn1cnnc1C1(F)CCN(c2c(C#N)cc(C(F)(F)F)nc2-c2cc3cn(C(C)(C)C)nc3nc2C(F)F)CC1] | C₂₆H₂₅F₆N₉ (M = 577.5 g/mol) ESI-MS: 578 [M + H]⁺ Rt (HPLC): 0.86 min (method J) | 8.78 (s, 1 H), 8.67 (s, 1 H), 8.47 (s, 1 H), 8.46 (s, 1 H), 7.19 (t, J = 53.2 Hz, 1 H), 3.68 (d, J = 1.4 Hz, 3 H), 3.35-3.43 (m, 2 H), 3.19-3.28 (m, 2 H), 2.03-2.25 (m, 4 H), 1.75 (s, 9 H) |
| 15 | 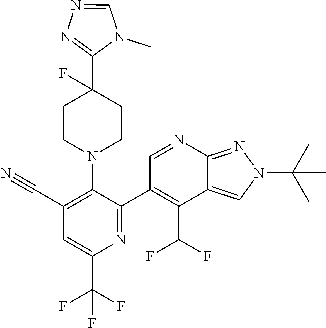 [SMILES: Cn1cnnc1C1(F)CCN(c2c(C#N)cc(C(F)(F)F)nc2-c2cnc3nn(C(C)(C)C)cc3c2C(F)F)CC1] | C₂₆H₂₅F₆N₉ (M = 577.5 g/mol) ESI-MS: 578 [M + H]⁺ Rt (HPLC): 0.86 min (method J) | 8.79 (s, 1 H), 8.46 (s, 1 H), 8.43-8.45 (m, 2 H), 6.95 (t, J = 53.5 Hz, 1 H), 3.66 (d, J = 1.5 Hz, 3 H), 3.32- 3.42 (m, 2 H), 3.11- 3.25 (m, 2 H), 2.05- 2.21 (m, 4 H), 1.74 (s, 9 H) |
| 16 | 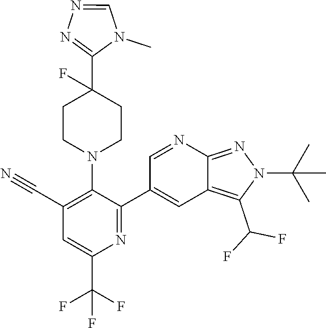 [SMILES: Cn1cnnc1C1(F)CCN(c2c(C#N)cc(C(F)(F)F)nc2-c2cnc3nn(C(C)(C)C)c(C(F)F)c3c2)CC1] | C₂₆H₂₅F₆N₉ (M = 577.5 g/mol) ESI-MS: 578 [M + H]⁺ Rt (HPLC): 0.96 min (method C) | 8.95 (d, J = 2.2 Hz, 1 H), 8.51 (d, J = 1.8 Hz, 1 H), 8.48 (s, 1 H), 8.38 (s, 1 H), 8.02 (t, J = 52.2 Hz, 1 H), 3.71 (d, J = 1.5 Hz, 3 H), 3.23-3.44 (m, 4 H), 2.16-2.34 (m, 4 H), 1.80 (s, 9 H) |
| 17 | 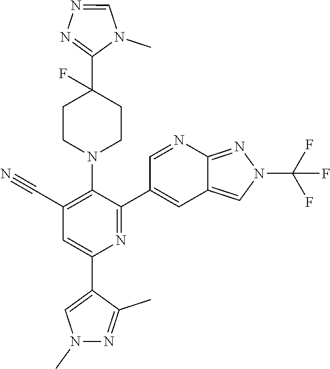 [SMILES: Cc1nn(C)cc1-c1cc(C#N)c(N2CCC(F)(c3nncn3C)CC2)c(-c2cnc3nn(C(F)(F)F)cc3c2)n1] | C₂₆H₂₃F₄N₁₁ (M = 565.5 g/mol) ESI-MS: 566 [M + H]⁺ Rt (HPLC): 0.70 min (method E) | 9.35 (s, 1 H), 9.17 (d, J = 2.3 Hz, 1 H), 8.53-8.57 (m, 2 H), 8.31 (s, 1 H), 8.01 (s, 1 H), 3.81 (s, 3 H), 3.73 (d, J = 1.5 Hz, 3 H), 3.28-3.38 (m, 2 H), 3.20-3.27 (m, 2 H), 2.43 (s, 3 H), 2.09-2.27 (m, 4 H) |
| 18 | 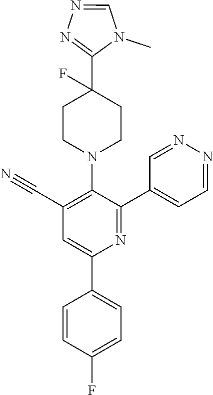 [SMILES: Cn1cnnc1C1(F)CCN(c2c(C#N)cc(-c3ccc(F)cc3)nc2-c2ccnnc2)CC1] | C₂₄H₂₀F₂N₈ (M = 458.5 g/mol) ESI-MS: 459 [M + H]⁺ Rt (HPLC): 0.85 min (method C) | 9.61 (dd, J = 2.2, 1.2 Hz, 1 H), 9.44 (dd, J = 5.3, 1.1 Hz, 1 H), 8.53 (s, 1 H), 8.50 (s, 1 H), 8.18-8.25 (m, 2 H), 8.03 (dd, J = 5.3, 2.3 Hz, 1 H), 7.30-7.40 (m, 2 H), 3.75 (d, J = 1.5 Hz, 3 H), 3.21-3.39 (m, 4 H), 2.13-2.31 (m, 4 H) |
| 19 | 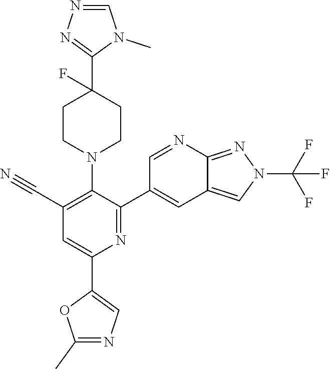 [SMILES: Cc1ncc(-c2cc(C#N)c(N3CCC(F)(c4nncn4C)CC3)c(-c3cnc4nn(C(F)(F)F)cc4c3)n2)o1] | C₂₅H₂₀F₄N₁₀O (M = 552.5 g/mol) ESI-MS: 553 [M + H]⁺ Rt (HPLC): 0.68 min (method H) | 9.35 (s, 1 H), 9.12 (d, J = 2.2 Hz, 1 H), 8.59 (s, 1 H), 8.56 (d, J = 2.3 Hz, 1 H), 8.17 (s, 1 H), 7.73 (s, 1 H), 3.73 (d, J = 1.5 Hz, 3 H), 3.25-3.35 (m, 4 H), 2.52 (s, 3 H), 2.08-2.27 (m, 4 H) |
| 20 | 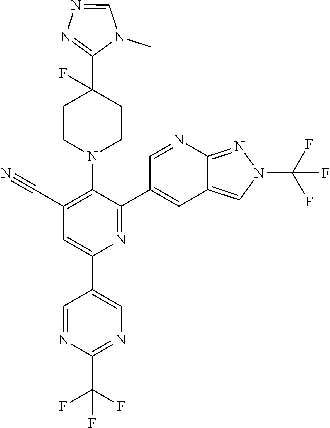 [SMILES: Cn1cnnc1C1(F)CCN(c2c(C#N)cc(-c3cnc(C(F)(F)F)nc3)nc2-c2cnc3nn(C(F)(F)F)cc3c2)CC1] | C₂₆H₁₈F₇N₁₁ (M = 617.5 g/mol) ESI-MS: 618 [M + H]⁺ Rt (HPLC): 0.83 min (method H) | 9.73 (s, 2 H), 9.37 (s, 1 H), 9.24 (d, J = 2.2 Hz, 1 H), 8.74 (s, 1 H), 8.68 (d, J = 2.2 Hz, 1 H), 8.55 (s, 1 H), 3.73 (d, J = 1.4 Hz, 3 H), 3.31-3.44 (m, 4 H), 2.14-2.35 (m, 4 H) |
| 21 | 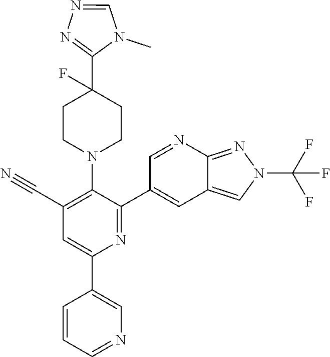 [SMILES: Cn1cnnc1C1(F)CCN(c2c(C#N)cc(-c3cccnc3)nc2-c2cnc3nn(C(F)(F)F)cc3c2)CC1] | C₂₆H₂₀F₄N₁₀ (M = 548.5 g/mol) ESI-MS: 549 [M + H]⁺ Rt (HPLC): 0.65 min (method F) | 9.35 (s, 1 H), 9.33 (dd, J = 2.3, 0.6 Hz, 1 H), 9.21 (d, J = 2.2 Hz, 1 H), 8.62-8.67 (m, 2 H), 8.57 (s, 1 H), 8.50 (dt, J = 8.3, 1.8 Hz, 1 H), 8.48 (s, 1 H), 7.54 (ddd, J = 8.0, 4.8, 0.6 Hz, 1 H), 3.72 (d, J = 1.5 Hz, 3 H), 3.32-3.38 (m, 4 H), 2.13-2.34 (m, 4 H) |
| 22 | 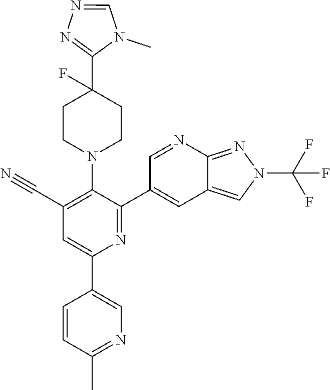 [SMILES: Cc1ccc(-c2cc(C#N)c(N3CCC(F)(c4nncn4C)CC3)c(-c3cnc4nn(C(F)(F)F)cc4c3)n2)cn1] | C₂₇H₂₂F₄N₁₀ (M = 562.5 g/mol) ESI-MS: 563 [M + H]⁺ Rt (HPLC): 0.70 min (method F) | 9.35 (s, 1 H), 9.18- 9.22 (m, 2 H), 8.63 (d, J = 2.3 Hz, 1 H), 8.52 (s, 1 H), 8.47 (s, 1 H), 8.39 (dd, J = 8.1, 2.4 Hz, 1 H), 7.39 (d, J = 8.1 Hz, 1 H), 3.72 (d, J = 1.5 Hz, 3 H), 3.30-3.38 (m, 4 H), 2.53 (s, 3 H), 2.12-2.31 (m, 4 H) |
| 23 | 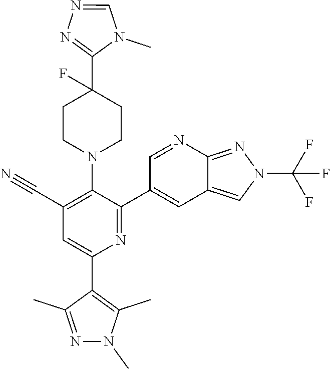 [SMILES: Cc1nn(C)c(C)c1-c1cc(C#N)c(N2CCC(F)(c3nncn3C)CC2)c(-c2cnc3nn(C(F)(F)F)cc3c2)n1] | C₂₇H₂₅F₄N₁₁ (M = 579.5 g/mol) ESI-MS: 580 [M + H]⁺ Rt (HPLC): 0.67 min (method G) | 9.35 (s, 1 H), 9.15 (d, J = 2.3 Hz, 1 H), 8.64 (s, 1 H), 8.53 (d, J = 2.2 Hz, 1 H), 7.80 (s, 1 H), 3.74 (d, J = 1.4 Hz, 3 H), 3.72 (s, 3 H), 3.22- 3.38 (m, 4 H), 2.41 (s, 3 H), 2.31 (s, 3 H), 2.10-2.27 (m, 4 H) |
| 24 | 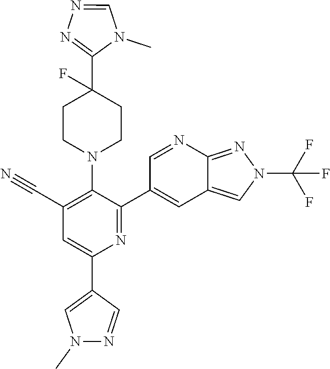 [SMILES: Cn1cc(-c2cc(C#N)c(N3CCC(F)(c4nncn4C)CC3)c(-c3cnc4nn(C(F)(F)F)cc4c3)n2)cn1] | C₂₅H₂₁F₄N₁₁ (M = 551.5 g/mol) ESI-MS: 552 [M + H]⁺ Rt (HPLC): 0.68 min (method E) | 9.34 (s, 1 H), 9.15 (d, J = 2.3 Hz, 1 H), 8.54 (d, J = 2.3 Hz, 1 H), 8.53 (s, 1 H), 8.34 (s, 1 H), 8.16 (s, 1 H), 8.07 (s, 1 H), 3.88 (s, 3 H), 3.72 (d, J = 1.5 Hz, 3 H), 3.19-3.38 (m, 4 H), 2.07-2.26 (m, 4 H) |
| 25 | 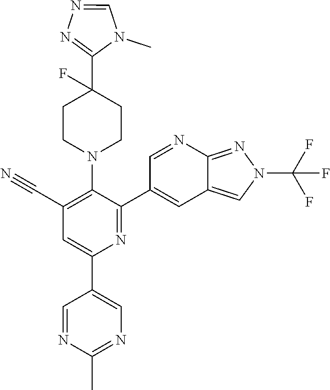 [SMILES: Cc1ncc(-c2cc(C#N)c(N3CCC(F)(c4nncn4C)CC3)c(-c3cnc4nn(C(F)(F)F)cc4c3)n2)cn1] | C₂₆H₂₁F₄N₁₁ (M = 563.5 g/mol) ESI-MS: 564 [M + H]⁺ Rt (HPLC): 0.65 min (method H) | 9.38 (s, 2 H), 9.35 (s, 1 H), 9.21 (d, J = 2.3 Hz, 1 H), 8.65 (d, J = 2.3 Hz, 1 H), 8.62 (s, 1 H), 8.60 (s, 1 H), 3.74 (d, J = 1.5 Hz, 3 H), 3.31-3.40 (m, 4 H), 2.69 (s, 3 H), 2.13-2.31 (m, 4 H) |
| 26 | 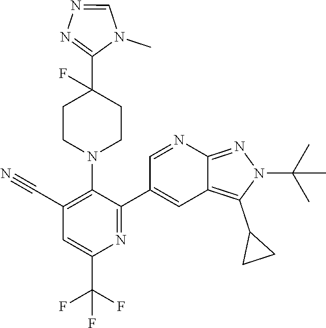 [SMILES: Cn1cnnc1C1(F)CCN(c2c(C#N)cc(C(F)(F)F)nc2-c2cnc3nn(C(C)(C)C)c(C4CC4)c3c2)CC1] | C₂₈H₂₉F₄N₉ (M = 567.6 g/mol) ESI-MS: 568 [M + H]⁺ Rt (HPLC): 0.93 min (method C) | 8.77 (d, J = 2.2 Hz, 1 H), 8.47 (s, 1 H), 8.39 (d, J = 2.2 Hz, 1 H), 8.32 (s, 1 H), 3.71 (d, J = 1.4 Hz, 3 H), 3.35-3.43 (m, 2 H), 3.23-3.29 (m, 2 H), 2.34-2.43 (m, 1 H), 2.15-2.34 (m, 4 H), 1.84 (s, 9 H), 1.17-1.25 (m, 2 H), 1.11 (s, 2 H) |

### Analytical HPLC methods

Method A

| | Vol % water | Vol % ACN | Flow |
|---|---|---|---|
| time (min) | (incl. 0.04% TFA) | (incl. 0.02% TFA) | [mL/min] |
| 0.00 | 95 | 5 | 1.5 |
| 0.70 | 5 | 95 | 1.5 |
| 1.16 | 5 | 95 | 1.5 |
| 1.50 | 95 | 5 | 1.5 |

Analytical column: Kinetex EVO C18_2.1×30 mm_5 μm; column temperature: 40° C.

Method B

| | Vol % water | Vol % | Flow |
|---|---|---|---|
| time (min) | (incl. 0.1% NH₃) | ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity;
Analytical column: XBridge (Waters) BEH C18_2.1 × 30 mm_2.5 μm;
column temperature: 60° C.

Method C

| | Vol % water | Vol % | Flow |
|---|---|---|---|
| time (min) | (incl. 0.1% FA) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200;
Analytical column: Sunfire C18_3.0 × 30 mm_2.5 μm;
column temperature: 60° C.

Method D

| | Vol % water | Vol % | Flow |
|---|---|---|---|
| time (min) | (incl. 0.1% TFA) | ACN | [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity;
Analytical column: Xbridge (Waters) BEH C18_2.1 × 30 mm_1.7 μm;
column temperature: 60° C.

Method E

| | Vol % water | Vol % | Flow |
|---|---|---|---|
| time (min) | (incl. 0.1% TFA) | ACN | [mL/min] |
| 0.00 | 95 | 5 | 1.5 |
| 1.30 | 0 | 100 | 1.5 |
| 1.50 | 0 | 100 | 1.5 |

Device description: Waters Acquity;
Analytical column: Sunfire (Waters) C18_3.0 × 30 mm_2.5 μm;
column temperature: 60° C.

Method F

| | Vol. % water | Vol. % | Flow |
|---|---|---|---|
| time (min) | (incl. 0.1% NH₃) | ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Xbridge (Waters) C18_3.0 × 30 mm_2.5 μm;
column temperature: 60° C.

Method G

| | Vol. % water | Vol % ACN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Sunfire (Waters) C18_3.0 × 30 mm_2.5 μm;
column temperature: 60° C.

Method H

| | Vol % water | Vol % ACN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Sunfire (Waters) C18_3.0 × 30 mm_2.5 μm;
column temperature: 60° C.

Method J

| | Vol. % water | Vol. % ACN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity;
Analytical column: Sunfire (Waters) C18_3.0 × 30 mm_2.5 μm (Waters);
column temperature: 60° C.

1. A compound of formula (I) 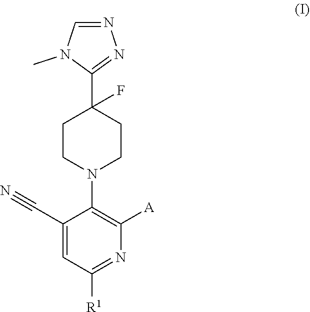
wherein
A is 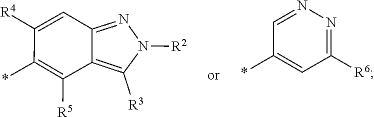
R¹ is selected from the group consisting of C₁₋₄-alkyl, F₁₋₉-fluoro-C₁₋₄-alkyl, C₃₋₆-cycloalkyl, F₁₋₈-fluoro-C₃₋₅-cycloalkyl,
or R¹ is selected from the group consisting of phenyl, pyridyl, pyrimidyl, pyrazolyl or oxazolyl; unsubstituted or substituted with one, two or three R⁷;
R² is selected from the group consisting of C₁₋₆-alkyl and F₁₋₉-fluoro-C₁₋₆-alkyl;
R³ is selected from the group consisting of H, C₁₋₆-alkyl, F₁₋₉-fluoro-C₁₋₆-alkyl and C₃₋₆-cycloalkyl;
R⁴ is selected from H or CHF₂;
R⁵ is selected from H or CHF₂;
R⁶ is selected from H or C₁₋₄-alkyl;
R⁷ is selected from C₁₋₄-alkyl, F₁₋₉-fluoro-C₁₋₆-alkyl or fluoro;
or a salt thereof, particularly a pharmaceutically acceptable salt thereof.

2. The compound of formula (I) according to claim 1, wherein A is 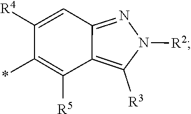
or a salt thereof.

3. The compound of formula (I) according to claim 1, wherein A is 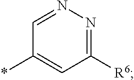
or a salt thereof.

4. The compound of formula (I) according to claim 1, wherein R¹ is selected from the group R1c, consisting of methyl, —CF₂CH₃, CF₃, cyclopropyl, 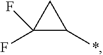
or R¹ is selected from the group consisting of 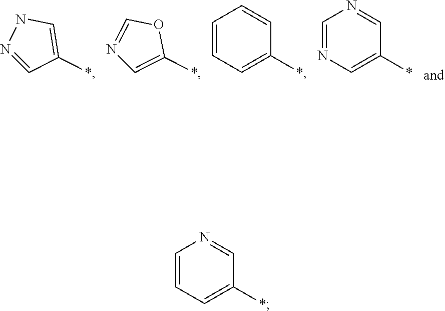
or a salt thereof.

5. The compound of formula (I) according to claim 1, wherein R² is selected from the group R2b, consisting of methyl, CF₃ and t-butyl;
or a salt thereof.

6. The compound of formula (I) according to claim 1, wherein R³ is selected from the group R3b, consisting of H, —CHF₂, CF₃ and cyclopropyl;
or a salt thereof.

7. The compound of formula (I) according to claim 1, wherein R⁶ is selected from the group R6b, consisting of H and methyl; or a salt thereof.

8. The compound of formula (I) according to claim 1, wherein R⁷ is selected from the group R7b, consisting of methyl, fluoro and CF₃; or a salt thereof.

9. The compound of formula (I) according to claim 1, having formula (1-a) 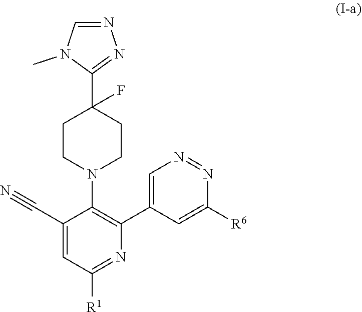
or a salt thereof.

10. The compound of formula (I) according to claim 1 having formula (1-b) 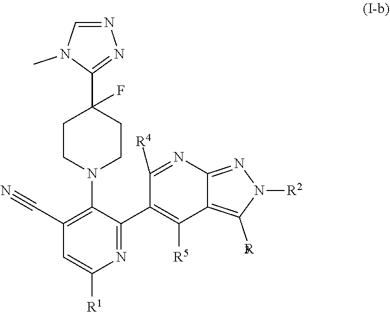
or a salt thereof.

11. The compound of formula (I) according to claim 1, selected from the group consisting of 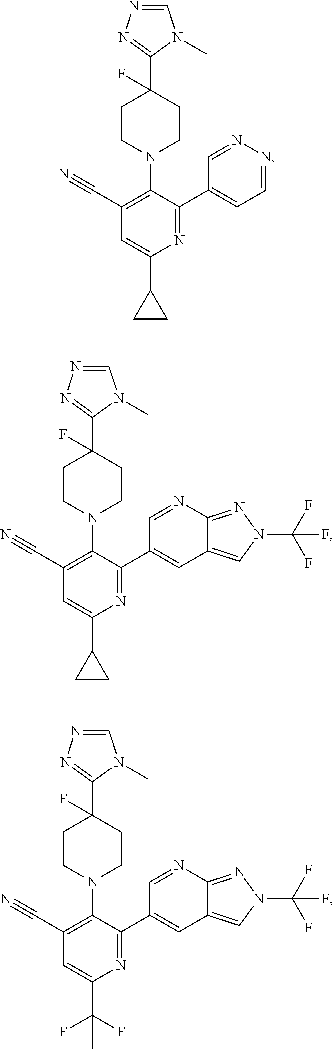 [SMILES: Cn1cnnc1C1(F)CCN(c2c(C#N)cc(C3CC3)nc2-c2ccnnc2)CC1 ; Cn1cnnc1C1(F)CCN(c2c(C#N)cc(C3CC3)nc2-c2cnc3nn(C(F)(F)F)cc3c2)CC1 ; Cn1cnnc1C1(F)CCN(c2c(C#N)cc(C(C)(F)F)nc2-c2cnc3nn(C(F)(F)F)cc3c2)CC1] 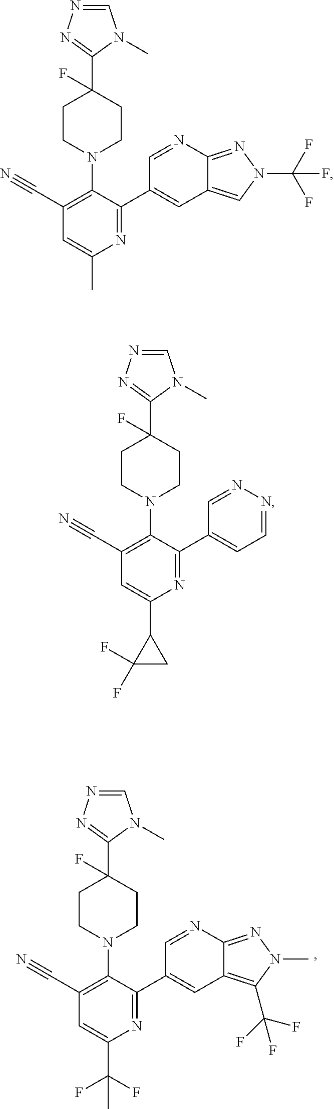 [SMILES: Cc1cc(C#N)c(N2CCC(F)(c3nncn3C)CC2)c(-c2cnc3nn(C(F)(F)F)cc3c2)n1 ; Cn1cnnc1C1(F)CCN(c2c(C#N)cc(C3CC3(F)F)nc2-c2ccnnc2)CC1 ; Cn1cnnc1C1(F)CCN(c2c(C#N)cc(C(C)(F)F)nc2-c2cnc3nn(C)c(C(F)(F)F)c3c2)CC1] 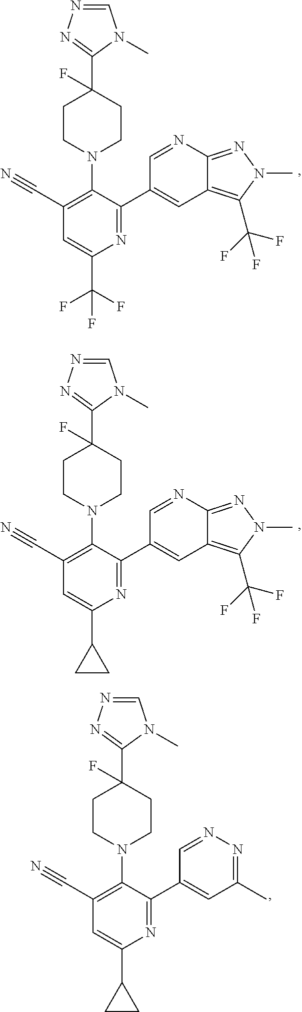 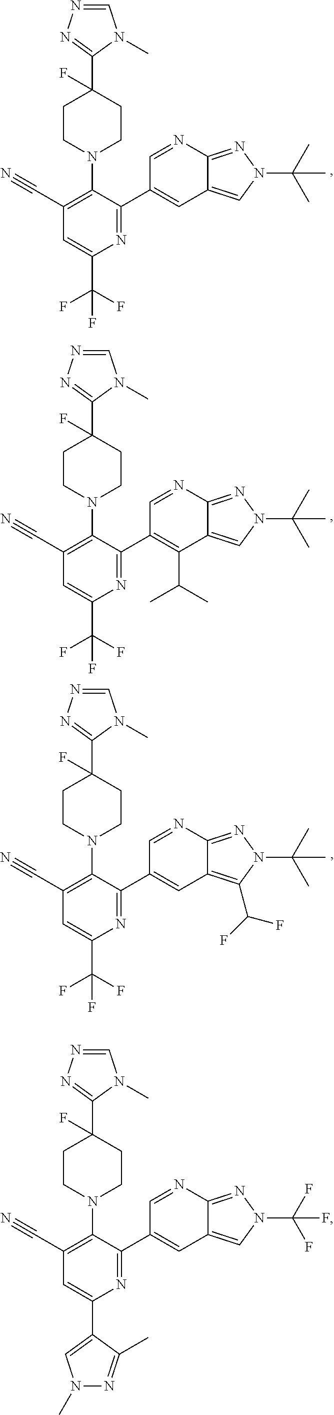 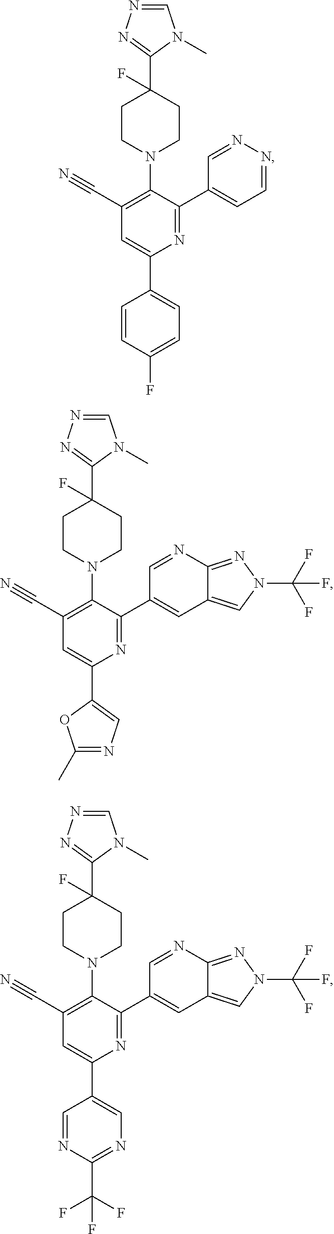 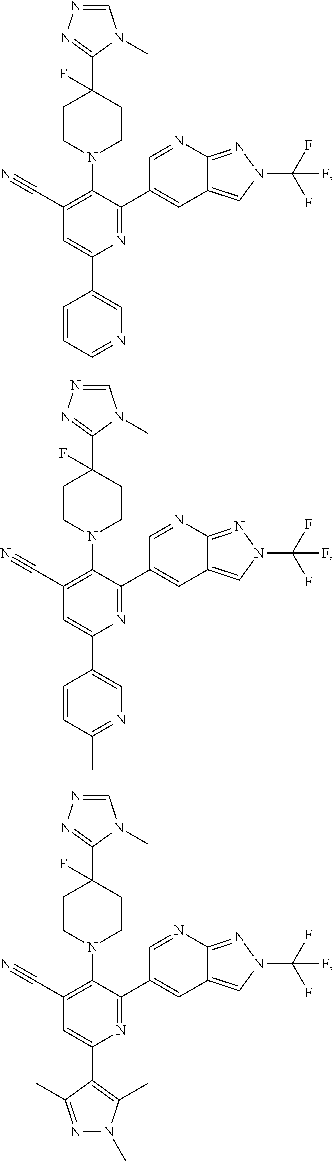 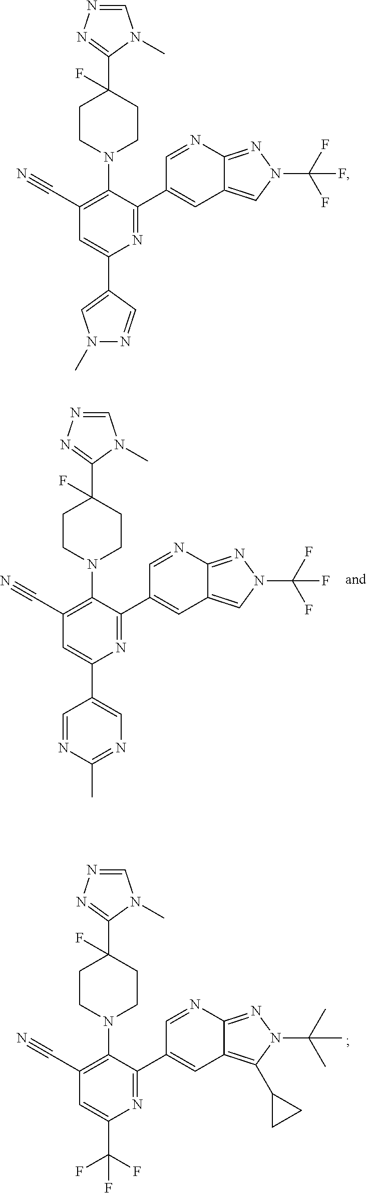
or a salt thereof.

12. A pharmaceutically acceptable salt of a compound according to claim 1.

13. A pharmaceutical composition comprising one or more compounds according to claim 1, or pharmaceutically acceptable salts thereof, together with one or more inert carriers and/or diluents.

14. A pharmaceutical composition comprising one or more compounds according to claim 1, or pharmaceutically acceptable salts thereof, and one or more additional therapeutic agents, together with one or more inert carriers and/or diluents.

15. The pharmaceutical composition according to claim 14 wherein the one or more additional therapeutic agents are selected from the group consisting of anticancer agents and antifibrotic agents.

16. (canceled)

17. A method for the treatment of a disease in a patient in need thereof, the comprising administering or more compounds according to claim 1 or pharmaceutically acceptable salts thereof to the patient.

18. The method according to claim 17, wherein the disease is selected from the group consisting of cancer, fibrotic diseases, neurodegenerative diseases, atherosclerosis, infectious diseases, and chronic kidney diseases.
